(19) United States
(12) Patent Application Publication
(10) Pub. No.: US 2013/0150376 A1
(43) Pub. Date: Jun. 13, 2013

(54) Novel Sultam Compounds

(71) Applicants: Michael Aaron Brodney, Newton, MA (US); Ivan Viktorovich Efremov, Chestnut Hill, MA (US); Christopher John Helal, Mystic, CT (US); Brian Thomas O'Neill, Haddam, CT (US)

(72) Inventors: Michael Aaron Brodney, Newton, MA (US); Ivan Viktorovich Efremov, Chestnut Hill, MA (US); Christopher John Helal, Mystic, CT (US); Brian Thomas O'Neill, Haddam, CT (US)

(73) Assignee: PFIZER INC., Groton, CT (US)

(21) Appl. No.: 13/638,642

(22) PCT Filed: Mar. 31, 2011
(86) PCT No.: PCT/IB01/51389

§ 371 Date: Oct. 1, 2012

Related U.S. Application Data
(60) Provisional application No. 61/322,406, filed on Apr. 9, 2010.
(60) Provisional application No. 61/450,398, filed on Mar. 8, 2011.

(51) Int. Cl. C07D 513/10; A61K 45/06; A61K 31/506; A61K 31/497; A61K 31/435; A61K 31/444

(52) U.S. Cl. CPC C07D 513/10; A61K 31/435; A61K 31/444; A61K 31/506; A61K 31/497; A61K 45/06
USPC 514/255.05; 546/19; 514/278; 544/230; 514/256

(57) ABSTRACT

Compounds and pharmaceutically acceptable salts of the compounds are disclosed, wherein the compounds have the structure of Formula I (I) as defined in the specification. Corresponding pharmaceutical compositions, methods of treatment, methods of synthesis, and intermediates are also disclosed.

### FIELD OF THE INVENTION

The present invention relates to the treatment of Alzheimer's disease and other neurodegenerative and/or neurological disorders in mammals, including humans. This invention also relates to inhibiting, in mammals, including humans, the production of A-beta peptides that contributes to the formation of neurological deposits of amyloid protein. More particularly, this invention relates to spiro-piperidine compounds useful for the treatment of neurodegenerative and/or neurological disorders, such as Alzheimer's disease and Down's Syndrome, related to A-beta peptide production.

### BACKGROUND OF THE INVENTION

Dementia results from a wide variety of distinctive pathological processes. The most common pathological processes causing dementia are Alzheimer's disease (AD), cerebral amyloid angiopathy (CM) and prion-mediated diseases (see, e.g., Haan et al., Clin. Neurol. Neurosurg. 1990, 92(4):305-310; Glenner ef al., J. Neurol. Sci. 1989, 94:1-28). AD affects nearly half of all people past the age of 85, the most rapidly growing portion of the United States population. As such, the number of AD patients in the United States is expected to increase from about 4 million to about 14 million by the middle of the next century. At present there are no effective treatments for halting, preventing, or reversing the progression of Alzheimer's disease. Therefore, there is an urgent need for pharmaceutical agents capable of slowing the progression of Alzheimer's disease and/or preventing it in the first place.

Several programs have been advanced by research groups to ameliorate the pathological processes causing dementia, AD, CM and prion-mediated diseases. Beta-secretase (BACE) inhibitors are one such strategy and numerous compounds are under evaluation by pharmaceutical groups. The present invention relates to a group of brain-penetrable BACE inhibitors and as such would be useful for the treatment of AD (see Ann. Rep. Med. Chem. 2007, Olsen et al., 42:27-47).

### SUMMARY OF THE INVENTION

The invention is directed to a compound, including the pharmaceutically acceptable salts thereof, having the structure of formula I:

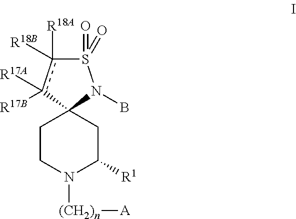

wherein the stereochemistry shown in formula I at the carbon bonded to R¹ and at the spirocyclic carbon is the absolute stereochemistry;

A is C₃₋₇cycloalkyl, C₆₋₁₀aryl, 4- to 10-membered heterocycloalkyl, or 5- to 10-membered heteroaryl; wherein said cycloalkyl, aryl, heterocycloalkyl or heteroaryl is optionally substituted with one to three R²;

R¹ is C₁₋₆alkyl, —(C(R¹⁹)₂)ₜ—C₃₋₇cycloalkyl, —(C(R¹⁹)₂)ₜ-(4- to 6-membered heterocycloalkyl), —(C(R¹⁹)₂)ₜ—C₆₋₁₀aryl, or —(C(R¹⁹)₂)ₜ-(5- to 6-membered heteroaryl); wherein said alkyl, cycloalkyl, heterocycloalkyl, aryl, or heteroaryl is optionally substituted with one to three halogen, cyano, C₃₋₆cycloalkyl, hydroxyl, —O—C₁₋₆alkyl, or —O—C₃₋₆cycloalkyl;

each R² is independently C₁₋₆alkyl, halogen, cyano, —COR³, —CON(R⁴)₂, —N(R⁴)COR³, —N(R⁴)CO₂R³, —N(R⁴)CON(R⁴)₂, —N(R⁴)SO₂R³, —SO₂R³, —SO₂N(R⁴)₂, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ-(5- to 10-membered heteroaryl), —(C(R¹⁹)₂)ₜ—N(R⁴)₂, or —(C(R¹⁹)₂)ₜ—OR⁵; wherein each R² alkyl, cycloalkyl, heterocycloalkyl, aryl, or heteroaryl is optionally independently substituted by one to three cyano, C₁₋₆alkyl, halogen, —CF₃ or —OR⁶;

each R³ is independently C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ(5- to 10-membered heteroaryl); wherein each R³ alkyl, cycloalkyl, heterocycloalkyl, aryl, or heteroaryl is optionally substituted with one to three d-ealkyl, halogen, cyano, hydroxyl, or —OR⁶;

each R⁴ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂), —C₆₋₁₀aryl, or —(C(R¹⁹)₂)ₜ-(5- to 10-membered heteroaryl); wherein each R⁴ alkyl, cycloalkyl, heterocycloalkyl, aryl, or heteroaryl is optionally independently substituted with one to three C₁₋₆alkyl, halogen, cyano, hydroxyl, or —OR⁶; or when two R⁴ substituents are attached to the same nitrogen atom they may be taken together with the nitrogen to which they are attached to form a 4- to 6-membered heterocycloalkyl;

each R⁵ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂), —C₆₋₁₀aryl, or —(C(R¹⁹)₂)ₜ-(5- to 10-membered heteroaryl); wherein each R⁵ alkyl, cycloalkyl, heterocycloalkyl, aryl, or heteroaryl is optionally substituted with one to three R⁷;

each R⁶ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂), —C₆₋₁₀aryl, or —(C(R¹⁹)₂)ₜ-(5- to 10-membered heteroaryl); wherein each R⁶ alkyl, cycloalkyl, heterocycloalkyl, aryl, or heteroaryl is optionally substituted with one to three R⁸;

each R⁷ is independently C₁₋₆alkyl, hydroxyl, —O—C₁₋₆alkyl, halogen, cyano, —(C(R¹⁹)₂)ₜN(R⁹)₂, —(C(R¹⁹)₂), —C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl);

each R⁸ is independently C₁₋₆alkyl, hydroxyl, —O—C₁₋₆alkyl, halogen, cyano, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl);

each R⁹ is independently hydrogen or C₁₋₃alkyl; or when two R⁹ substituents are attached to the same nitrogen atom they may be taken together with the nitrogen to which they are attached to form a 4- to 5-membered heterocycloalkyl;

B is C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ-(5- to 10-membered heteroaryl); wherein each B alkyl, cycloalkyl, heterocycloalkyl, aryl, or heteroaryl is optionally substituted with one to three R¹⁰;

each R¹⁰ is independently halogen, C₁₋₆alkyl, cyano, hydroxyl, —O—C₁₋₆alkyl, —O—C₃₋₆cycloalkyl, —CO(C₁₋₆alkyl), —CON(R¹¹)₂, —N(R¹¹)CO(C₁₋₆alkyl), —N(R¹¹)SO₂(C₁₋₆alkyl), —SO₂(C₁₋₆alkyl), —SO₂N(R¹¹)₂, —N(R¹¹)₂, —NR¹¹CON(R¹¹)₂, —NR¹¹COOC₁₋₆alkyl, —(C(R¹⁹)₂), —C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ(5- to 10-membered heteroaryl); wherein each R¹⁰ alkyl, cycloalkyl, heterocycloalkyl, aryl, or heteroaryl is optionally independently substituted with one to three R¹²;

each R¹¹ is independently hydrogen or C₁₋₆alkyl; or when two R¹¹ substituents are attached to the same nitrogen atom they may be taken together with the nitrogen to which they are attached to form a 4- to 6-membered heterocycloalkyl;

each R¹² is independently C₁₋₆alkyl, halogen, cyano, hydroxyl, —O—C₁₋₆alkyl, —O—C₃₋₆cycloalkyl, —CO(C₁₋₆alkyl), —CON(R¹¹)₂, —(C(R¹⁹)₂)ₜN(R¹³)₂, —N(R¹¹)CO(C₁₋₆alkyl), —N(R¹¹)CO₂(C₁₋₆alkyl), —NR¹¹CON(R¹¹)₂, —N(R¹¹)SO₂(C₁₋₆alkyl), —SO₂(C₁₋₆alkyl), —SO₂N(R¹¹)₂, —(C(R¹⁹)₂)ₜOR¹⁴, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜC₆₋₁₀aryl, or —(C(R¹⁹)₂)ₜ-(5- to 10-membered heteroaryl); wherein each R¹² alkyl, cycloalkyl, heterocycloalkyl, aryl, or heteroaryl is optionallyindependently substituted by one to three cyano, C₁₋₆alkyl, halogen, —CF₃ or —OR¹⁵;

each R¹³ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl); wherein each R¹³ alkyl, cycloalkyl, heterocycloalkyl, aryl, or heteroaryl is optionally independently substituted with one to three cyano, C₁₋₆alkyl, halogen, —CF₃, or —OR¹⁵; or when two R¹³ substituents are attached to the same nitrogen atom they may be taken together with the nitrogen to which they are attached to form a 4- to 6-membered heterocycloalkyl;

each R¹⁴ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl); wherein each R¹⁴ alkyl, cycloalkyl, heterocycloalkyl, aryl, or heteroaryl is optionally substituted with one to three cyano, C₁₋₆alkyl, halogen, —CF₃, or —OR¹⁵;

each R¹⁵ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ(5- to 10-membered heteroaryl); wherein each R¹⁵ alkyl, cycloalkyl, heterocycloalkyl, aryl, or heteroaryl is optionally substituted with one to three R⁸;

when [symbol] is a single bond, R^{17A} and R^{17B} are independently hydrogen, hydroxyl, or C₁₋₆alkyl wherein said alkyl is optionally substituted with fluorine, —SO₂(C₁₋₃alkyl), —SO₂(C₃₋₆cycloalkyl), cyano, NR¹¹COO(C₁₋₃alkyl), hydroxyl, —O—C₁₋₆alkyl, or —O—C₃₋₆cycloalkyl; or R^{17A} and R^{17B} together with the carbon to which they are bonded form a C═O, C₃₋₆cycloalkyl, or 4- to 6-membered heterocycloalkyl; and R^{18A} and R^{18B} are independently hydrogen, C₁₋₆alkyl, —(C(R¹⁹)₂)ₜ—C₃₋₇cycloalkyl, —(C(R¹⁹)₂)ₜ(4- to 6-membered heterocycloalkyl), —(C(R¹⁹)₂)ₜ—C₆₋₁₀aryl, —(C(R¹⁹)₂)ₜ-(5- to 6-membered heteroaryl), —(C(R¹⁹)₂), —OR¹⁶, —(C(R¹⁹)₂)ₜN(R¹¹)₂, —(C(R¹⁹)₂), —CO(C₁₋₆alkyl), —(C(R¹⁹)₂)ₜ—CON(R¹¹)₂, —(C(R¹⁹)₂)ₜ—N(R¹¹)CONR¹¹, —(C(R¹⁹)₂)ₜ—SO₂(C₁₋₆alkyl), or —(C(R¹⁹)₂)ₜ—CO₂R³; or R^{18A} and R^{18B} together with the carbon to which they are bonded form a C₃₋₆cycloalkyl or a 4- to 5-membered heterocycloalkyl, wherein said cycloalkyl or heterocycloalkyl is optionally substituted with one to two fluorine, C₁₋₆alkyl, cyano, —CF₃, C₃₋₆cycloalkyl, hydroxyl, —O—C₁₋₆alkyl, or —O—C₃₋₆cycloalkyl;

each R¹⁶ is independently hydrogen, C₁₋₆alkyl, C₃₋₅cycloalkyl, 4- to 6-membered heterocycloalkyl, C₆₋₁₀aryl, or 5- to 6-membered heteroaryl, wherein said alkyl, cycloalkyl, heterocycloalkyl, aryl, or heteroaryl is optionally substituted with one to three halogen or —CF₃;

or R^{17A} and R^{18A}, together with the carbons to which they are bonded, can form a C₃₋₆cycloalkyl or 4- to 6-membered heterocycloalkyl; wherein said cycloalkyl or heterocycloalkyl are optionally substituted with one to three C₁₋₆alkyl, fluorine, cyano, hydroxyl, —O—C₁₋₆alkyl, or —O—C₃₋₆cycloalkyl;

when [symbol] is a double bond, R^{17B} is absent and R^{17A} is hydrogen, —(C(R¹⁹)₂)ₜN(R¹⁸)₂, —(C(R¹⁹)₂)ₜ—OR¹⁶, or C₁₋₆alkyl wherein said alkyl is optionally substituted with one to three fluorine; and R^{18B} is absent and R^{18A} is hydrogen, hydroxyl, cyano, —(C(R¹⁹)₂)ₜC₃₋₆cycloalkyl, —(C(R¹⁹)₂)ₜ-(4- to 6-membered heterocycloalkyl), —(C(R¹⁹)₂)ₜ—C₆₋₁₀aryl, —(C(R¹⁹)₂)ₜ-(5- to 6-membered heteroaryl), fluorine, C₁₋₆alkyl, —(C(R¹⁹)₂)ₜ—SO₂(C₁₋₆alkyl), —(C(R¹⁹)₂)ₜ—SO₂N(R¹¹)₂, —(C(R¹⁹)₂)ₜ—CON(R¹¹)₂, —(C(R¹⁹)₂)ₜ—COO(C₁₋₆alkyl), —(C(R¹⁹)₂)ₜ—C(O)(C₁₋₆alkyl), —(C(R¹⁹)₂)ₜ—N(R¹¹)₂, —(C(R¹⁹)₂)ₜ—NR¹¹CO(C₁₋₆alkyl), —(C(R¹⁹)₂)ₜ—N(R¹¹)CO₂(C₁₋₆alkyl), —(C(R¹⁹)₂)ₜ—NR¹¹CON(R¹¹)₂, or —(C(R¹⁹)₂)ₜ—N(R¹¹)SO₂(C₁₋₆alkyl); wherein said alkyl, cycloalkyl, heterocycloalkyl, aryl or heteroaryl substituent is optionally substituted with one to three halogen, cyano, —CF₃, C₁₋₆alkyl, hydroxyl, —O—C₁₋₆alkyl, or —O—C₃₋₆cycloalkyl;

or R^{17A} and R^{18A}, together with the carbons to which they are bonded, can form a fused C₅₋₆cycloalkyl, 4- to 6-membered heterocycloalkyl, 6- to 10-membered aryl or a 5- to 6-membered heteroaryl ring; wherein said cycloalkyl, heterocycloalkyl, aryl, or heteroaryl are optionally substituted with one to three C₁₋₆alkyl, halogen, cyano, —CF₃, hydroxyl, —O—C₁₋₆alkyl, or —O—C₃₋₆cycloalkyl;

each R19 is independently hydrogen, C₁₋₆alkyl, or CF₃;

n is an integer independently selected from 1, 2 and 3;

each t is an integer independently selected from 0, 1, 2 and 3.

In one embodiment of the invention [symbol] is a single bond, and R^{17A} and R^{17B} are both hydrogen, and R^{18A} and R^{18B} are both hydrogen.

In another embodiment of the invention [symbol] is a single bond, and R^{17A} and R^{17B} are both hydrogen; and R^{18A} is hydrogen and R^{18B} is C₁₋₆alkyl.

In another embodiment of the invention [symbol] is a single bond, and R^{17A} and R^{17B} together with the carbon to which they are bonded form a C═O; and R^{18A} and R^{18B} are both C₁₋₆alkyl.

In another embodiment of the invention [symbol] is a single bond, and R^{17A} is hydrogen and R^{17B} is —OH; and R^{18A} and R^{18B} are both hydrogen.

In another embodiment, [symbol] is a double bond, R^{17B} is absent and R^{17A} is hydrogen; and R^{18B} is absent and R^{18A} is hydrogen.

In another embodiment, [symbol] is a double bond, R^{17B} is absent and R^{17A} is —(C(R¹⁹)₂)ₜN(R¹⁶)₂, wherein t is zero and one R¹⁶ is hydrogen and the other R¹⁶ is alkyl; and R^{18B} is absent and R^{18A} is hydrogen.

In another embodiment, [symbol] is a double bond, R^{17B} is absent and R^{17A} is —(C(R¹⁹)₂)ₜOR¹⁶, wherein t is zero and R¹⁶ is C₁₋₃alkyl; and R^{18B} is absent and R^{18A} is hydrogen.

In any of the embodiments described above, A is C₃₋₇cycloalkyl.

In another embodiment of the invention, A is 4- to 10-membered heterocycloalkyl.

In any of the embodiments described above, A is C₆₋₁₀aryl.

In any of the embodiments described above, A is 5- to 10-membered heteroaryl.

In any of the embodiments described above, A is C₃₋₇cycloalkyl, C₆₋₁₀aryl, 4- to 10-membered heterocycloalkyl, or 5- to 10-membered heteroaryl and A is optionally substituted with one R² substituent selected from the group consisting of C₁₋₆alkyl, halogen, cyano, —COR³, —CON(R⁴)₂, —N(R⁴)COR³, —N(R⁴)CO₂R³, —N(R⁴)CON(R⁴)₂, —N(R⁴)SO₂R³, —SO₂R³, —SO₂N(R⁴)₂, —(C(R¹⁹)₂)ₜC₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ-(5- to 10-membered heteroaryl), —(C(R¹⁹)₂)ₜN(R⁴)₂, or —(C(R¹⁹)₂)ₜ—OR⁵; wherein each R² alkyl, cycloalkyl, heterocycloalkyl, aryl, or heteroaryl is optionally substituted by one to three cyano, C₁₋₆alkyl, halogen, —CF₃ or —OR⁶.

In another embodiment, A is C₆₋₁₀aryl and A is optionally substituted with one R² substituent selected from the group consisting of halogen, C₁₋₆alkyl, —(C(R¹⁹)₂)ₜ—OR⁵, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl); wherein said cycloalkyl, heterocycloalkyl, aryl, or heteroaryl, is optionally substituted by one to three C₁₋₆alkyl or halogen.

In another embodiment, A is 5- to 10-membered heteroaryl, and A is optionally substituted with one R² substituent selected from the group consisting of halogen, C₁₋₆alkyl, —(C(R¹⁹)₂)ₜ—OR⁵, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl); wherein said cycloalkyl, heterocycloalkyl, aryl, or heteroaryl is optionally substituted by one to three C₁₋₆alkyl or halogen.

In another embodiment, A is C₆₋₁₀aryl and is substituted with one R² and R² is —(C(R¹⁹)₂), —OR⁵, wherein R⁵ is —(C(R¹⁹)₂)ₜC₃₋₇cycloalkyl or —(C(R¹⁹)₂)ₜ-(5- to 10-membered heteroaryl), t is zero, and said R⁵ cycloalkyl or heteroaryl is optionally substituted with one to three R⁷.

In another embodiment, A is C₆₋₁₀aryl and is substituted with one R² and R² is —(C(R¹⁹)₂)ₜ—C₆₋₁₀aryl wherein t is zero and the R² aryl is optionally substituted by one to three cyano, C₁₋₆alkyl, halogen, —CF₃, or —OR⁶.

In another embodiment, A is C₆₋₁₀aryl and is substituted with one R² and R² is —(C(R¹⁹)₂)ₜ-(5- to 10-membered heteroaryl) wherein t is zero and the R² heteroaryl is optionally substituted by one to three cyano, C₁₋₆alkyl, halogen, —CF₃, or —OR⁶.

In another embodiment, A is 5- to 10-membered heteroaryl and is substituted with one R² and R² is —(C(R¹⁹)₂), —OR⁵, wherein R⁵ is —(C(R¹⁹)₂)ₜ—C₃₋₇cycloalkyl or —(C(R¹⁹)₂)ₜ-(5- to 10-membered heteroaryl), t is zero, and said R⁵ cycloalkyl or heteroaryl is optionally substituted with one to three R⁷.

In another embodiment, A is a 5- to 10-membered heteroaryl and is substituted with one R² and R² is —(C(R¹⁹)₂)ₜ—C₆₋₁₀aryl wherein t is zero and the R² aryl is optionally substituted by one to three cyano, C₁₋₆alkyl, halogen, —CF₃, or —OR⁶.

In another embodiment, A is a 5- to 10-membered heteroaryl and is substituted with one R² and R² is —(C(R¹⁹)₂)ₜ-(5- to 10-membered heteroaryl) wherein t is zero and the R² heteroaryl is optionally substituted by one to three cyano, C₁₋₆alkyl, halogen, —CF₃, or —OR⁶.

In another embodiment of the invention A is C₃₋₇cycloalkyl, C₆₋₁₀aryl, 4- to 10-membered heterocycloalkyl, or 5- to 10-membered heteroaryl and A is optionally substituted with two R² wherein each R² substituent is selected from the group consisting of C₁₋₆alkyl, halogen, cyano, —COR³, —CON(R⁴)₂, —N(R⁴)COR³, —N(R⁴)CO₂R³, —N(R⁴)CON(R⁴)₂, —N(R⁴)SO₂R³, —SO₂R³, —SO₂N(R⁴)₂, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ-(5- to 10-membered heteroaryl), —(C(R¹⁹)₂)ₜ—N(R⁴)₂, or —(C(R¹⁹)₂)ₜOR⁵; and each R² alkyl, cycloalkyl, heterocycloalkyl, aryl, or heteroaryl is optionally independently substituted by one to three cyano, C₁₋₆alkyl, halogen, —CF₃ or —OR⁶. In another embodiment, A is C₃₋₇cycloalkyl, C₆₋₁₀aryl, 4- to 10-membered heterocycloalkyl, or 5- to 10-membered heteroaryl and A is optionally substituted with two R² wherein each R² is alkyl optionally independently substituted by one to three cyano, C₁₋₆alkyl, halogen, —CF₃, or —OR⁶.

In another embodiment, A is C₆₋₁₀aryl or 5- to 10-membered heteroaryl, and A is optionally substituted with two R² substituents wherein each R² substituent is selected from the group consisting of C₁₋₆alkyl, halogen, cyano, —COR³, —CON(R⁴)₂, —N(R⁴)COR³, —N(R⁴)CO₂R³, —N(R⁴)CON(R⁴)₂, —N(R⁴)SO₂R³, —SO₂R³, —SO₂N(R⁴)₂, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ-(5- to 10-membered heteroaryl), —(C(R¹⁹)₂)ₜ—N(R⁴)₂, or —(C(R¹⁹)₂), —OR⁵; and each R² alkyl, cycloalkyl, heterocycloalkyl, aryl, or heteroaryl is optionally independently substituted by one to three cyano, C₁₋₆alkyl, halogen, —CF₃ or —OR⁶.

In another embodiment, A is C₆₋₁₀aryl or 5- to 10-membered heteroaryl and A is optionally substituted with two R² wherein each R² is C₁₋₆alkyl optionally independently substituted by one to three cyano, C₁₋₆alkyl, fluorine, —CF₃, or —OR⁶. In another embodiment, A is C₃₋₁₀aryl or 5- to 10-membered heteroaryl, and A is optionally substituted with two R² substituents and each R² is independently C₁₋₆alkyl, halogen, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ-(5- to 10-membered heteroaryl), wherein each R² cycloalkyl, heterocycloalkyl, aryl, or heteroaryl is optionally independently substituted by one to three cyano, C₁₋₆alkyl, halogen, —CF₃, or —OR⁶. In another embodiment, A is C₆₋₁₀aryl and A is optionally substituted with two R² substituents and at least one R² is —(C(R¹⁹)₂)ₜ—C₆₋₁₀aryl, wherein t is zero and the R² aryl is optionally substituted with one to three cyano, C₁₋₆alkyl, halogen, —CF₃, or —OR⁶. In another embodiment, A is C₆₋₁₀aryl and A is optionally substituted with two R² substituents and at least one R² is —(C(R¹⁹)₂)ₜ—OR⁵, wherein t is zero; and pharmaceutically acceptable salts thereof. In another example of this embodiment, A is C₆₋₁₀aryl and A is optionally substituted with two R² substituents and each R² is —(C(R¹⁹)₂)ₜ—OR⁵, wherein t is zero. In another example of this embodiment, A is 5- to 10-membered heteroaryl and A is optionally substituted with two R² substituents, and at least one R² is —(C(R¹⁹)₂)ₜ—C₆₋₁₀aryl, wherein t is zero and the R² aryl is optionally substituted with one to three cyano, C₁₋₆alkyl, halogen, —CF₃, or —OR⁶. In another embodiment, A is C₆₋₁₀aryl and A is optionally substituted with two R² wherein one R² is —(C(R¹⁹)₂)ₜ—OR⁵ wherein t is zero and R⁵ is H, and the other R² is —(C(R¹⁹)₂)ₜ—C₃₋₇cycloalkyl or —(C(R¹⁹)₂)ₜ-(4- to 10-membered heterocycloalkyl) and the R² cycloalkyl or heterocycloalkyl is optionally substituted by one to three cyano, C₁₋₆alkyl, halogen, —CF₃ or —OR⁶. In another embodiment, A is C₆₋₁₀aryl and A is optionally substituted with two R² wherein one R² is —(C(R¹⁹)₂)ₜ—OR⁵ wherein t is zero and R⁵ is H, and the other R² is —(C(R¹⁹)₂)ₜ-(5- to 10-membered heteroaryl) optionally substituted by one to three cyano, C₁₋₆alkyl, halogen, —CF₃ or —OR⁶. In another example of this embodiment, A is 5- to 10-membered heteroaryl and A is optionally substituted with two R² substituents and at least one R² is —(C(R¹⁹)₂)ₜ—OR⁵, wherein t is zero. In another example of this embodiment, A is 5- to 10-membered heteroaryl and A is optionally substituted with two R² substituents and each R² is —(C(R¹⁹)₂)ₜ—OR⁵, wherein t is zero.

In another embodiment of the invention A is C₃₋₇cycloalkyl, C₆₋₁₀aryl, 4- to 10-membered heterocycloalkyl, or 5- to 10-membered heteroaryl and A is optionally substituted with three R² wherein each R² substituent is selected from the group consisting of C₁₋₆alkyl, halogen, cyano, —COR³, —CON(R⁴)₂, —N(R⁴)COR³; —N(R⁴)CO₂R³, —N(R⁴)CON(R⁴)₂, —N(R⁴)SO₂R³, —SO₂R³, —SO₂N(R⁴)₂, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ-(5- to 10-membered heteroaryl), —(C(R¹⁹)₂)ₜ—N(R⁴)₂, or —(C(R¹⁹)₂), —OR⁵; and each R² alkyl, cycloalkyl, heterocycloalkyl, aryl, or heteroaryl is optionally independently substituted by one to three cyano, C₁₋₆alkyl, halogen, —CF₃ or —OR⁶. In another embodiment, A is C₆₋₁₀aryl or 5- to 10-membered heteroaryl and A is optionally substituted with three R² wherein each R² substituent is selected from the group consisting of C₁₋₆alkyl, halogen, cyano, —COR³, —CON(R⁴)₂, —N(R⁴)COR³, —N(R⁴)CO₂R³, —N(R⁴)CON(R⁴)₂, —N(R⁴)SO₂R³, —SO₂R³, —SO₂N(R⁴)₂, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ(5- to 10-membered heteroaryl), —(C(R¹⁹)₂), —N(R⁴)₂, or —(C(R¹⁹)₂)ₜ—OR⁵; and each R² aryl or heteroaryl is optionally independently substituted by one to three cyano, C₁₋₆alkyl, halogen, —CF₃ or —OR⁶. In another embodiment, A is C₆₋₁₀aryl or 5- to 10-membered heteroaryl, and A is optionally substituted with three R² substituents, and each R² is C₁₋₆alkyl optionally independently substituted by one to three cyano, C₁₋₆alkyl, fluorine, —CF₃, or —OR⁶. In another embodiment, A is C₆₋₁₀aryl or 5- to 10-membered heteroaryl, and A is optionally substituted with three R² substituents, and each R² is independently halogen, —(C(R¹⁹)₂)ₜ—OR⁵, cyano, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl), wherein each R² alkyl, cycloalkyl, heterocycloalkyl, aryl, or heteroaryl is optionally independently substituted by one to three cyano, C₁₋₆alkyl, halogen, —CF₃, or —OR⁶. In another embodiment, A is C₆₋₁₀aryl or 5- to 10-membered heteroaryl, and A is optionally substituted with three R² substituents and at least one R² is —(C(R¹⁹)₂)ₜ-(4- to 10-membered heterocycloalkyl), wherein t is zero, and the heterocycloalkyl is pyrrolidinyl, piperidinyl, or morpholinyl, and the heterocycloalkyl is optionally substituted by cyano, C₁₋₆alkyl, halogen, —CF₃, or —OR⁶.

In any of the embodiments described above, B is —(C(R¹⁹)₂)ₜC₆₋₁₀aryl or —(C(R¹⁹)₂)ₜ-(5- to 10-membered heteroaryl); wherein each B aryl or heteroaryl is optionally substituted with one to three R10. In any of the embodiments described above, B is —(C(R¹⁹)₂)ₜ—C₆₋₁₀aryl optionally substituted with one to three R¹⁰. In any of the embodiments described above, B is phenyl substituted with one fluorine. In any of the embodiments described above, B is —(C(R¹⁹)₂)ₜ-(5- to 10-membered heteroaryl) optionally substituted with one to three R10. In any of the embodiments described above, B is pyridine. In any of the embodiments described above, B is pyridine substituted with one methyl. In any of the embodiments described above, B is pyrazine. In any of the embodiments described above, B is pyrazine substituted with one methyl. In any of the embodiments described above, B is pyrimidine. In any of the embodiments described above, B is pyrimidine substituted with one methyl. In any of the embodiments described above, B is pyridazine. In any of the embodiments described above, B is oxadiazole. In any of the embodiments described above, B is oxadiazole substituted with one methyl. In any of the embodiments described above, B is thiadiazole. In any of the embodiments described above, B is thiadiazole substituted with one methyl. In any of the embodiments described above, B is oxazole. In any of the embodiments described above, B is oxazole substituted with one methyl. In any of the embodiments described above, B is thiazole. In any of the embodiments described above, B is thiazole substituted with one methyl. In any of the embodiments described above, B is triazole. In any of the embodiments described above, B is triazole substituted with one methyl. In any of the embodiments described above, R¹ is C₁₋₆alkyl. In any of the embodiments described above, R¹ is C₁₋₆alkyl, substituted with —O—C₁₋₆alkyl

In any of the embodiments described above, n is one.

Examples of the invention include:
2-isopropoxy-4-{[(5R,7S)-7-methyl-1-(6-methylpyridin-2-yl)-2,2-dioxido-2-thia-1,8-diazaspiro[4.5]dec-8-yl]methyl}phenol;
2-cyclopropyl-4-{[(5R,7S)-7-methyl-1-(6-methylpyridin-2-yl)-2,2-dioxido-2-thia-1,8-diazaspiro[4.5]dec-8-yl]methyl}phenol;
2-(4-methylisothiazol-3-yl)-4-{[(5R,7S)-7-methyl-1-(6-methylpyridin-2-yl)-2,2-dioxido-2-thia-1,8-diazaspiro[4.5]dec-8-yl]methyl}phenol;
2-cyclobutyl-4-{[(5R,7S)-7-methyl-1-(6-methylpyridin-2-yl)-2,2-dioxido-2-thia-1,8-diazaspiro[4.5]dec-8-yl]methyl}phenol;
4-{[(5R,7S)-7-methyl-1-(6-methylpyridin-2-yl)-2,2-dioxido-2-thia-1,8-diazaspiro[4.5]dec-8-yl]methyl}-2-(5-methyl-1,3-thiazol-4-yl)phenol;
4-{[(5R,7S)-7-methyl-1-(6-methylpyridin-2-yl)-2,2-dioxido-2-thia-1,8-diazaspiro[4.5]dec-8-yl]methyl}-2-(3-methylpyridin-2-yl)phenol;
4-{[(5R,7S)-7-methyl-1-(6-methylpyridin-2-yl)-2,2-dioxido-2-thia-1,8-diazaspiro[4.5]dec-8-yl]methyl}-2-(5-methyl-1,3-oxazol-4-yl)phenol;
2-isopropoxy-4-{[(5R,7S)-7-methyl-1-(6-methylpyridin-2-yl)-2,2-dioxido-2-thia-1,8-diazaspiro[4.5]dec-3-en-8-yl]methyl}phenol;
2-cyclopropyl-4-{[(5R,7S)-7-methyl-1-(6-methylpyridin-2-yl)-2,2-dioxido-2-thia-1,8-diazaspiro[4.5]dec-3-en-8-yl]methyl}phenol;
2-(4-methylisothiazol-3-yl)-4-{[(5R,7S)-7-methyl-1-(6-methylpyridin-2-yl)-2,2-dioxido-2-thia-1,8-diazaspiro[4.5]dec-3-en-8-yl]methyl}phenol;
2-cyclobutyl-4-{[(5R,7S)-7-methyl-1-(6-methylpyridin-2-yl)-2,2-dioxido-2-thia-1,8-diazaspiro[4.5]dec-3-en-8-yl]methyl}phenol;
4-{[(5R,7S-7-methyl-1-(6-methylpyridin-2-yl)-2,2-dioxido-2-thia-1,8-diazaspiro[4.5]dec-3-en-8-yl]methyl}-2-(5-methyl-1,3-thiazol-4-yl)phenol;
4-{[(5R,7S)-7-methyl-1-(6-methylpyridin-2-yl)-2,2-dioxido-2-thia-1,8-diazaspiro[4.5]dec-3-en-8-yl]methyl}-2-(3-methylpyridin-2-yl)phenol;
4-{[(5R,7S)-7-methyl-1-(6-methylpyridin-2-yl)-2,2-dioxido-2-thia-1,8-diazaspiro[4.5]dec-3-en-8-yl]methyl}-2-(5-methyl-1,3-oxazol-4-yl)phenol; and pharmaceutically

acceptable salts thereof.

Other examples of the invention include:
2-isopropoxy-4-{[(5R,7S)-7-methyl-2,2-dioxido-1-pyrazin-2-yl-2-thia-1,8-diazaspiro[4.5]dec-8-yl]methyl}phenol;
2-cyclopropyl-4-{[(5R,7S)-7-methyl-2,2-dioxido-1-pyrazin-2-yl-2-thia-1,8-diazaspiro[4.5]dec-8-yl]methyl}phenol;
4-{[(5R,7S)-7-methyl-2,2-dioxido-1-pyrazin-2-yl-2-thia-1,8-diazaspiro[4.5]dec-8-yl]methyl}-2-(4-methylisothiazol-3-yl)phenol;
2-cyclobutyl-4-{[(5R,7S)-7-methyl-2,2-dioxido-1-pyrazin-2-yl-2-thia-1,8-diazaspiro[4.5]dec-8-yl]methyl}phenol;
4-{[(5R,7S)-7-methyl-2,2-dioxido-1-pyrazin-2-yl-2-thia-1,8-diazaspiro[4.5]dec-8-yl]methyl}-2-(5-methyl-1,3-thiazol-4-yl)phenol;
4-{[(5R,7S)-7-methyl-2,2-dioxido-1-pyrazin-2-yl-2-thia-1,8-diazaspiro[4.5]dec-8-yl]methyl}-2-(3-methylpyridin-2-yl)phenol;
4-{[(5R,7S)-7-methyl-2,2-dioxido-1-pyrazin-2-yl-2-thia-1,8-diazaspiro[4.5]dec-8-yl]methyl}-2-(5-methyl-1,3-oxazol-4-yl)phenol;
2-isopropoxy-4-{[(5R,7S)-7-methyl-2,2-dioxido-1-pyrazin-2-yl-2-thia-1,8-diazaspiro[4.5]dec-3-en-8-yl]methyl}phenol;
2-cyclopropyl-4-{[(5R,7S)-7-methyl-2,2-dioxido-1-pyrazin-2-yl-2-thia-1,8-diazaspiro[4.5]dec-3-en-8-yl]methyl}phenol;
4-{[(5R,7S)-7-methyl-2,2-dioxido-1-pyrazin-2-yl-2-thia-1,8-diazaspiro[4.5]dec-3-en-8-yl]methyl}-2-(4-methylisothiazol-3-yl)phenol;
2-cyclobutyl-4-{[(5R,7S)-7-methyl-2,2-dioxido-1-pyrazin-2-yl-2-thia-1,8-diazaspiro[4.5]dec-3-en-8-yl]methyl}phenol;
4-{[(5R,7S)-7-methyl-2,2-dioxido-1-pyrazin-2-yl-2-thia-1,8-diazaspiro[4.5]dec-3-en-8-yl]methyl}-2-(5-methyl-1,3-thiazol-4-yl)phenol;
4-{[(5R,7S)-7-methyl-2,2-dioxido-1-pyrazin-2-yl-2-thia-1,8-diazaspiro[4.5]dec-3-en-8-yl]methyl}-2-(3-methylpyridin-2-yl)phenol;
4-{[(5R,7S)-7-methyl-2,2-dioxido-1-pyrazin-2-yl-2-thia-1,8-diazaspiro[4.5]dec-3-en-8-yl]methyl}-2-(5-methyl-1,3-oxazol-4-yl)phenol; and pharmaceutically acceptable salts thereof.

Additional examples of the invention include:
2-isopropoxy-4-{[(5R,7S)-7-methyl-1-(6-methylpyrazin-2-yl)-2,2-dioxido-2-thia-1,8-diazaspiro[4.5]dec-8-yl]methyl}phenol;
2-cyclopropyl-4-{[(5R,7S)-7-methyl-1-(6-methylpyrazin-2-yl)-2,2-dioxido-2-thia-1,8-diazaspiro[4.5]dec-8-yl]methyl}phenol;
2-(4-methylisothiazol-3-yl)-4-{[(5R,7S)-7-methyl-1-(6-methylpyrazin-2-yl)-2,2-dioxido-2-thia-1,8-diazaspiro[4.5]dec-8-yl]methyl}phenol;
2-cyclobutyl-4-{[(5RJS)-7-methyl-1-(6-methylpyrazin-2-yl)-2,2-dioxido-2-thia-18-diazaspiro[4.5]dec-8-yl]methyl}phenol;
4-{[(5R,7S)-7-methyl-1-(6-methylpyrazin-2-yl)-2,2-dioxido-2-thia-1,8-diazaspiro[4.5]dec-8-yl]methyl}-2-(5-methyl-1,3-thiazol-4-yl)phenol;
4-{[(5R,7S)-7-methyl-1-(6-methylpyrazin-2-yl)-2,2-dioxido-2-thia-1,8-diazaspiro[4.5]dec-8-yl]methyl}-2-(3-methylpyridin-2-yl)phenol;
4-{[(5R,7S)-7-methyl-1-(6-methylpyrazin-2-yl)-2,2-dioxido-2-thia-1,8-diazaspiro[4.5]dec-8-yl]methyl}-2-(5-methyl-1,3-oxazol-4-yl)phenol;
2-isopropoxy-4-{[(5R,7S)-7-methyl-1-(6-methylpyrazin-2-yl)-2,2-dioxido-2-thia-1,8-diazaspiro[4.5]dec-3-en-8-yl]methyl}phenol;
2-cyclopropyl-4-{[(5R,7S)-7-methyl-1-(6-methylpyrazin-2-yl)-2,2-dioxido-2-thia-1,8-diazaspiro[4.5]dec-3-en-8-yl]methyl}phenol;
2-(4-methylisothiazol-3-yl)-4-{[(5R,7S)-7-methyl-1-(6-methylpyrazin-2-yl)-2,2-dioxido-2-thia-1,8-diazaspiro[4.5]dec-3-en-8-yl]methyl}phenol;
2-cyclobutyl-4-{[(5R,7S)-7-methyl-1-(6-methylpyrazin-2-yl)-2,2-dioxido-2-thia-1,8-diazaspiro[4.5]dec-3-en-8-yl]methyl}phenol;
4-{[(5R,7S)-7-methyl-1-(6-methylpyrazin-2-yl)-2,2-dioxido-2-thia-1,8-diazaspiro[4.5]dec-3-en-8-yl]methyl}-2-(5-methyl-1,3-thiazol-4-yl)phenol;
4-{[(5R,7S)-7-methyl-1-(6-methylpyrazin-2-yl)-2,2-dioxido-2-thia-1,8-diazaspiro[4.5]dec-3-en-8-yl]methyl}-2-(3-methylpyridin-2-yl)phenol;
4-{[(5R,7S)-7-methyl-1-(6-methylpyrazin-2-yl)-2,2-dioxido-2-thia-1,8-diazaspiro[4.5]dec-3-en-8-yl]methyl}-2-(5-methyl-1,3-oxazol-4-yl)phenol; and pharmaceutically acceptable salts thereof.

Preferred embodiments include
4-{[(5R,7S)-1-(3-Fluorophenyl)-7-methyl-2,2-dioxido-2-thia-1,8-diazaspiro[4.5]dec-8-yl]methyl}-2-isopropoxyphenol;
6-{[(5R,7S)-1-(3-Fluorophenyl)-7-methyl-2,2-dioxido-2-thia-1,8-diazaspiro[4.5]dec-8-yl]methyl}-4-isopropoxypyridin-3-ol;
4-{[(5R,7S)-1-(3-fluorophenyl)-7-methyl-2,2-dioxido-2-thia-1,8-diazaspiro[4.5]dec-3-en-8-yl]methyl}-2-(3-methyl-2-thienyl)phenol, hydrochloride salt;
2′-ethyl-5-{[(5R,7S)-1-(3-fluorophenyl)-7-methyl-2,2-dioxido-2-thia-1,8-diazaspiro[4.5]dec-3-en-8-yl]methyl}biphenyl-2-ol;
2-cyclopentyl-4-{[(5R,7S)-1-(3-fluorophenyl)-7-methyl-2,2-dioxido-2-thia-1,8-diazaspiro[4.5]dec-3-en-8-yl]methyl}phenol;
2′-ethyl-5-{[(5R,7S)-1-(3-fluorophenyl)-7-methyl-2,2-dioxido-2-thia-1,8-diazaspiro[4.5]dec-8-yl]methyl}biphenyl-2-ol;
4-{[(5R,7S)-1-(3-fluorophenyl)-7-methyl-2,2-dioxido-2-thia-1,8-diazaspiro[4.5]dec-3-en-8-yl]methyl}-2-isopropoxyphenol;
4-{[(5R,7S)-1-(3-fluorophenyl)-7-methyl-2,2-dioxido-2-thia-1,8-diazaspiro[4.5]dec-3-en-8-yl]methyl}-2-(trifluoromethoxy)phenol, hydrochloride salt;
4-{[(5R,7S)-1-(3-fluorophenyl)-7-methyl-2,2-dioxido-2-thia-1,8-diazaspiro[4.5]dec-3-en-8-yl]methyl}-2-(tetrahydrofuran-2-yl)phenol;
(5R,7S)-1-(3-fluorophenyl)-8-[(5-isobutyl-1,3-oxazol-4-yl)methyl]-7-methyl-2-thia-1,8-diazaspiro[4.5]dec-3-ene 2,2-dioxide;
2-(cyclopropyloxy)-4-{[(5R,7S)-1-(3-fluorophenyl)-7-methyl-2,2-dioxido-2-thia-1,8-diazaspiro[4.5]dec-3-en-8-yl]methyl}phenol, hydrochloride salt;
2-(cyclopropyloxy)-4-{[(5R,7S)-1-(3-fluorophenyl)-7-methyl-2,2-dioxido-2-thia-1,8-diazaspiro[4.5]dec-3-en-8-yl]methyl}phenol;
2-chloro-4-{[(5R,7S)-1-(3-fluorophenyl)-7-methyl-2,2-dioxido-2-thia-1,8-diazaspiro[4.5]dec-3-en-8-yl]methyl}phenol, hydrochloride salt;
4-{[(5R,7S)-1-(3-fluorophenyl)-7-methyl-2,2-dioxido-2-thia-1,8-diazaspiro[4.5]dec-3-en-8-yl]methyl}-2-(trifluoromethyl)phenol, hydrochloride;
2-fluoro-4-{[(5R,7S)-1-(3-fluorophenyl)-7-methyl-2,2-dioxido-2-thia-1,8-diazaspiro[4.5]dec-3-en-8-yl]methyl}phenol, hydrochloride salt;
(5R,7S)-8-{[4-(cyclobutylmethyl)-1,3-thiazol-5-yl]methyl}-1-(3-fluorophenyl)-7-methyl-2-thia-1,8-diazaspiro[4.5]dec-3-ene 2,2-dioxide;
(5R,7S)-1-(3-fluorophenyl)-7-methyl-8-[(5-pyridin-3-yl-1,3-oxazol-4-yl)methyl]-2-thia-1,8-diazaspiro[4.5]dec-3-ene 2,2-dioxide, hydrochloride salt;
(5R,7S)-8-{[4-(cyclopropylmethyl)-1,3-thiazol-5-yl]methyl}-1-(3-fluorophenyl)-7-methyl-2-thia-1,8-diazaspiro[4.5]dec-3-ene-2,2-dioxide, hydrochloride salt;
5-{[(5R,7S)-1-(3-fluorophenyl)-7-methyl-2,2-dioxido-2-thia-1,8-diazaspiro[4.5]dec-3-en-8-yl]methyl}-2′-methylbiphenyl-2-ol, hydrochloride salt;
2-ethoxy-4-{[(5R,7S)-1-(3-fluorophenyl)-7-methyl-2,2-dioxido-2-thia-1,8-diazaspiro[4.5]dec-3-en-8-yl]methyl}phenol;
4-{[(5R,7S)-1-(3-fluorophenyl)-7-methyl-2,2-dioxido-2-thia-1,8-diazaspiro[4.5]dec-3-en-8-yl]methyl}-2-(tetrahydrofuran-2-yl)phenol;
4-{[(5R,7S)-1-(3-fluorophenyl)-7-methyl-2,2-dioxido-2-thia-1,8-diazaspiro[4.5]dec-3-en-8-yl]methyl}-2-(tetrahydrofuran-2-yl)phenol;
4-{[(5R,7S)-1-(3-fluorophenyl)-7-methyl-2,2-dioxido-2-thia-1,8-diazaspiro[4.5]dec-8-yl]methyl}-2-(tetrahydrofuran-2-yl)phenol;
(5R,7S)-1-(3-fluorophenyl)-7-methyl-8-[(2′-methylbiphenyl-3-yl)methyl]-2-thia-1,8-diazaspiro[4.5]decane 2,2-dioxide, formate salt;
(5R,7S)-1-(3-fluorophenyl)-7-methyl-8-[(2′-methylbiphenyl-3-yl)methyl]-2-thia-1,8-diazaspiro[4.5]dec-3-ene 2,2-dioxide;
4-{[(5R,7S)-1-(3-fluorophenyl)-7-methyl-2,2-dioxido-2-thia-1,8-diazaspiro[4.5]dec-8-yl]methyl}-2-(tetrahydrofuran-2-yl)phenol;
5-{[(5R,7S)-1-(3-fluorophenyl)-7-methyl-2,2-dioxido-2-thia-1,8-diazaspiro[4.5]dec-8-yl]methyl}-2′-methylbiphenyl-2-ol, hydrochloride salt;
4-{[(5R,7S)-1-(3,4-difluorophenyl)-7-methyl-2,2-dioxido-2-thia-1,8-diazaspiro[4.5]dec-3-en-8-yl]methyl}-2-isopropoxyphenol;
4-{[(5R,7S)-1-(3-fluorophenyl)-7-methyl-2,2-dioxido-2-thia-1,8-diazaspiro[4.5]dec-3-en-8-yl]methyl}-2-(4-methyl-1,2-thiazol-3-yl)phenol, trifluoroacetic acid salt;
4-{[(5R,7S)-1-(3-fluorophenyl)-7-methyl-2,2-dioxido-2-thia-1,8-diazaspiro[4.5]dec-3-en-8-yl]methyl}-2-(5-methyl-1,3-thiazol-4-yl)phenol, ammonium salt;
4-{[(5R,7S)-1-(3-fluorophenyl)-7-methyl-2,2-dioxido-2-thia-1,8-diazaspiro[4.5]dec-3-en-8-yl]methyl}-2-(3-methylpyridin-2-yl)phenol;
2-cyclobutyl-4-{[(5R,7S)-1-(3-fluorophenyl)-7-methyl-2,2-dioxido-2-thia-1,8-diazaspiro[4.5]dec-3-en-8-yl]methyl}phenol;
(5R,7S)-1-(3-fluorophenyl)-8-[4-hydroxy-3-(4-methylisothiazol-3-yl)benzyl]-7-methyl-2-thia-1,8-diazaspiro[4.5]decan-4-one 2,2-dioxide;
(5R,7S)-1-(3-fluorophenyl)-8-[4-hydroxy-3-(4-methylisothiazol-3-yl]benzyl]-7-methyl-2-thia-1,8-diazaspiro[4.5]decan-4-one 2,2-dioxide;
(5R,7S)-8-(3-cyclobutyl-4-hydroxybenzyl)-1-(3-fluorophenyl)-7-methyl-2-thia-1,8-diazaspiro[4.5]decan-4-one 2,2-dioxide;
4-{[(5R,7S)-1-(3-fluorophenyl)-4-methoxy-7-methyl-2,2-dioxido-2-thia-1,8-diazaspiro[4.5]dec-3-en-8-yl]methyl}-2-(4-methylisothiazol-3-yl)phenol, ammonium salt;
4-{[(5R,7S)-7-methyl-2,2-dioxido-1-(pyrazin-2-yl)-2-thia-1,8-diazaspiro[4.5]dec-8-yl]methyl}-2-(4-methylisothiazol-3-yl)phenol.

It is understood that descriptions of any one substituent, such as R¹, may be combined with descriptions of any other substituents, such as R², such that each and every combination of the first substituent and the second substituent is provided herein the same as if each combination were specifically and individually listed. For example, in one variation, R¹ is taken together with R² to provide an embodiment wherein R¹ is methyl and R² is halogen.

It will be understood that the compounds of formula I, and pharmaceutically acceptable salts thereof, also include hydrates, solvates and polymorphs of said compounds of formula I, and pharmaceutically acceptable salts thereof, as discussed below.

In one embodiment, the invention also relates to each of the individual compounds described as Examples 1 to 92 in the Examples section of the subject application, as well as the examples listed above (including the free bases or pharmaceutically acceptable salts thereof).

In another embodiment the present invention provides methods of treating neurological and psychiatric disorders comprising: administering to a patient in need thereof an amount of a compound of formula I effective in treating such disorders. Neurological and psychiatric disorders, include but are not limited to: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, AIDS-induced dementia, vascular dementia, mixed dementias, age-associated memory impairment, Alzheimer's disease, Huntington's Chorea, amyotrophic lateral sclerosis, ocular damage, retinopathy, cognitive disorders, including cognitive disorders associated with schizophrenia and bipolar disorders, idiopathic and drug-induced Parkinson's disease, muscular spasms and disorders associated with muscular spasticity including tremors, epilepsy, convulsions, migraine, migraine headache, urinary incontinence, substance tolerance, substance withdrawal, withdrawal from opiates, nicotine, tobacco products, alcohol, benzodiazepines, cocaine, sedatives, and hypnotics, psychosis, mild cognitive impairment, amnestic cognitive impairment, multi-domain cognitive impairment, obesity, schizophrenia, anxiety, generalized anxiety disorder, social anxiety disorder, panic disorder, post-traumatic stress disorder, obsessive compulsive disorder, mood disorders, depression, mania, bipolar disorders, trigeminal neuralgia, hearing loss, tinnitus, macular degeneration of the eye, emesis, brain edema, pain, acute and chronic pain states, severe pain, intractable pain, neuropathic pain, post-traumatic pain, tardive dyskinesia, sleep disorders, narcolepsy, attention deficit/hyperactivity disorder, autism, Asperger's disease, and conduct disorder in a mammal, comprising administering to the mammal an effective amount of compound of formula I or pharmaceutically acceptable salt thereof. Accordingly, in one embodiment, the invention provides a method for treating a condition in a mammal, such as a human, selected from the conditions above, comprising administering a compound of formula I to the mammal. The mammal is preferably a mammal in need of such treatment. As examples, the invention provides a method for treating attention deficit/hyperactivity disorder, schizophrenia and Alzheimer's Disease.

In another embodiment the present invention provides methods of treating neurological and psychiatric disorders comprising: administering to a patient in need thereof an amount of a compound of formula I effective in treating such disorders. The compound of formula I is optionally used in combination with another active agent. Such an active agent may be, for example, an atypical antipsychotic, a cholinesterase inhibitor, or NMDA receptor antagonist. Such atypical antipsychotics include, but are not limited to, ziprasidone, clozapine, olanzapine, risperidone, quetiapine, aripiprazole, paliperidone; such NMDA receptor antagonists include but are not limited to memantine; and such cholinesterase inhibitors include but are not limited to donepezil and galantamine.

The invention is also directed to a pharmaceutical composition comprising a compound of formula I, and a pharmaceutically acceptable carrier.

The term “alkyl” refers to a linear or branched-chain saturated hydrocarbyl substituent (i.e., a substituent obtained from a hydrocarbon by removal of a hydrogen) containing from one to twenty carbon atoms; in one embodiment from one to twelve carbon atoms; in another embodiment, from one to ten carbon atoms; in another embodiment, from one to six carbon atoms; and in another embodiment, from one to four carbon atoms. Examples of such substituents include methyl, ethyl, propyl (including n-propyl and isopropyl), butyl (including n-butyl, isobutyl, sec-butyl and tert-butyl), pentyl, isoamyl, hexyl and the like. In some instances, the number of carbon atoms in a hydrocarbyl substituent (i.e., alkyl, alkenyl, cycloalkyl, aryl, etc.) is indicated by the prefix “C_{x-y},” wherein x is the minimum and y is the maximum number of carbon atoms in the substituent. Thus, for example, “C₁₋₆alkyl” refers to an alkyl substituent containing from 1 to 6 carbon atoms.

“Alkenyl” refers to an aliphatic hydrocarbon having at least one carbon-carbon double bond, including straight chain, branched chain or cyclic groups having at least one carbon-carbon double bond. Preferably, it is a medium size alkenyl having 2 to 6-carbon atoms. For example, as used herein, the term “C₂₋₆alkenyl” means straight or branched chain unsaturated radicals of 2 to 6 carbon atoms, including, but not limited to ethenyl, 1-propenyl, 2-propenyl(allyl), isopropenyl, 2-methyl-1-propenyl, 1-butenyl, 2-butenyl, and the like; optionally substituted by 1 to 5 suitable substituents as defined above such as fluoro, chloro, trifluoromethyl, (C₁-C₆)alkoxy, (C₆-C₁₀)aryloxy, trifluoromethoxy, difluoromethoxy or C₁₋₆alkyl. When the compounds of the invention contain a C₂₋₆alkenyl group, the compound may exist as the pure E (entgegen) form, the pure Z (zusammen) form, or any mixture thereof.

“Alkylidene” refers to a divalent group formed from an alkane by removal of two hydrogen atoms from the same carbon atom, the free valencies of which are part of a double bond.

“Alkynyl” refers to an aliphatic hydrocarbon having at least one carbon-carbon triple bond, including straight chain, branched chain or cyclic groups having at least one carbon-carbon triple bond. Preferably, it is a lower alkynyl having 2 to 6 carbon atoms. For example, as used herein, the term “C₂₋₆alkynyl” is used herein to mean a straight or branched hydrocarbon chain alkynyl radical as defined above having 2 to 6 carbon atoms and one triple bond.

The term “cycloalkyl” refers to a carbocyclic substituent obtained by removing a hydrogen from a saturated carbocyclic molecule and having three to fourteen carbon atoms. In one embodiment, a cycloalkyl substituent has three to ten carbon atoms. Examples of cycloalkyl include cyclopropyl, cyclobutyl, cyclopentyl and cyclohexyl.

The term “cycloalkyl” also includes substituents that are fused to a C₆-C₁₀ aromatic ring or to a 5- to 10-membered heteroaromatic ring, wherein a group having such a fused cycloalkyl group as a substituent is bound to a carbon atom of the cycloalkyl group. When such a fused cycloalkyl group is substituted with one or more substituents, the one or more substituents, unless otherwise specified, are each bound to a carbon atom of the cycloalkyl group. The fused C₆-C₁₀ aromatic ring or 5- to 10-membered heteroaromatic ring may be optionally substituted with halogen, C₁₋₆alkyl, C₃₋₁₀ cycloalkyl, or ═O.

A cycloalkyl may be a single ring, which typically contains from 3 to 6 ring atoms. Examples include cyclopropyl, cyclobutyl, cyclopentyl, and cyclohexyl. Alternatively, 2 or 3 rings may be fused together, such as bicyclodecanyl and decalinyl.

The term “aryl” refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C₄₋₁₀carbocyclic ring, such as a C₅ or a C₆ carbocyclic ring, or to a 4- to 10-membered heterocyclic ring, wherein a group having such a fused aryl group as a substituent is bound to an aromatic carbon of the aryl group. When such a fused aryl group is substituted with one more substituents, the one or more substituents, unless otherwise specified, are each bound to an aromatic carbon of the fused aryl group. The fused C₄₋₁₀ carbocyclic or 4- to 10-membered heterocyclic ring may be optionally substituted with halogen, C₁₋₆alkyl, C₃₋₁₀cycloalkyl, or ═O. Examples of aryl groups include accordingly phenyl, naphthalenyl, tetrahydronaphthalenyl(also known as “tetralinyl”), indenyl, isoindenyl, indanyl, anthracenyl, phenanthrenyl, benzonaphthenyl (also known as “phenalenyl”), and fluorenyl.

In some instances, the number of atoms in a cyclic substituent containing one or more heteroatoms (i.e., heteroaryl or heterocycloalkyl) is indicated by the prefix “x- to y-membered”, wherein x is the minimum and y is the maximum number of atoms forming the cyclic moiety of the substituent. Thus, for example, 5- to 8-membered heterocycloalkyl refers to a heterocycloalkyl containing from 5 to 8 atoms, including one or more heteroatoms, in the cyclic moiety of the heterocycloalkyl.

The term “hydroxy” or “hydroxyl” refers to —OH. When used in combination with another term(s), the prefix “hydroxy” indicates that the substituent to which the prefix is attached is substituted with one or more hydroxy substituents. Compounds bearing a carbon to which one or more hydroxy substituents are attached include, for example, alcohols, enols and phenol.

The term “cyano” (also referred to as “nitrile”) means —CN, which also may be depicted:

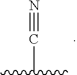 [SMILES: *C#N]

The term “halogen” refers to fluorine (which may be depicted as —F), chlorine (which may be depicted as —Cl), bromine (which may be depicted as —Br), or iodine (which may be depicted as —I). In one embodiment, the halogen is chlorine. In another embodiment, the halogen is fluorine. In another embodiment, the halogen is bromine.

The term “heterocycloalkyl” refers to a substituent obtained by removing a hydrogen from a saturated or partially saturated ring structure containing a total of 4 to 14 ring atoms, wherein at least one of the ring atoms is a heteroatom selected from oxygen, nitrogen, or sulfur. For example, as used herein, the term “4- to 10-membered heterocycloalkyl” means the substituent is a single ring with 4 to 10 total members. A heterocycloalkyl alternatively may comprise 2 or 3 rings fused together, wherein at least one such ring contains a heteroatom as a ring atom (i.e., nitrogen, oxygen, or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom.

The term “heterocycloalkyl” also includes substituents that are fused to a C₆₋₁₀ aromatic ring or to a 5- to 10-membered heteroaromatic ring, wherein a group having such a fused heterocycloalkyl group as a substituent is bound to a heteroatom of the heterocycloalkyl group or to a carbon atom of the heterocycloalkyl group. When such a fused heterocycloalkyl group is substituted with one or more substituents, the one or more substituents, unless otherwise specified, are each bound to a heteroatom of the heterocycloalkyl group or to a carbon atom of the heterocycloalkyl group. The fused C₆-C₁₀ aromatic ring or 5- to 10-membered heteroaromatic ring may be optionally substituted with halogen, C₁₋₆alkyl, C₃₋₁₀cycloalkyl, C₁₋₆alkoxy, or ═O.

The term “heteroaryl” refers to an aromatic ring structure containing from 5 to 14 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents include but are not limited to: 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused ring substituents such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heteroaryl” also includes pyridyl N-oxides and groups containing a pyridine N-oxide ring.

Examples of single-ring heteroaryls and heterocycloalkyls include but are not limited to furanyl, dihydrofuranyl, tetrahydrofuranyl, thiophenyl (also known as “thiofuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, isoxazolinyl, thiazolyl, isothiazolyl, thiazolinyl, isothiazolinyl, thiazolidinyl, isothiazolidinyl, thiadiazolyl, oxathiazolyl, oxadiazolyl (including oxadiazolyl, 1,2,4-oxadiazolyl (also known as “azoximyl”), 1,2,5-oxadiazolyl (also known as “furazanyl”), or 1,3,4-oxadiazolyl), pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), morpholinyl, azepinyl, oxepinyl, thiepinyl, and diazepinyl.

Examples of 2-fused-ring heteroaryls and heterocycloalkyls include but are not limited to indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl, indolyl, isoindolyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

Examples of 3-fused-ring heteroaryls or heterocycloalkyls include but are not limited to 5,6-dihydro-4H-imidazo[4,5,1-ij]quinoline, 4,5-dihydroimidazo[4,5,1-hi]indole, 4,5,6,7-tetrahydroimidazo[4,5,1-jk][1]benzazepine, and dibenzofuranyl.

Other examples of fused-ring heteroaryls include but are not limited to benzo-fused heteroaryls such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, or 3,1,4-benzoxazinyl), benzisoxazinyl (including 1,2-benzisoxazinyl or 1,4-benzisoxazinyl), carbazolyl, xanthenyl, and acridinyl.

The term “heteroaryl” also includes substituents such as pyridyl and quinolinyl that are fused to a C₄₋₁₀ carbocyclic ring, such as a C₅ or a C₆ carbocyclic ring, or to a 4- to 10-membered heterocyclic ring, wherein a group having such a fused heteroaryl group as a substituent is bound to an aromatic carbon of the heteroaryl group or to a heteroatom of the heteroaryl group. When such a fused heteroaryl group is substituted with one or more substituents, the one or more substituents, unless otherwise specified, are each bound to an aromatic carbon of the heteroaryl group or to a heteroatom of the heteroaryl group. The fused C₄₋₁₀ carbocyclic or 4- to 10-membered heterocyclic ring may be optionally substituted with halogen, C₁₋₆alkyl, C₃₋₁₀ cycloalkyl, or ═O.

Additional examples of heteroaryls and heterocycloalkyls include but are not limited to: 3-1H-benzimidazol-2-one, (1-substituted)-2-oxo-benzimidazol-3-yl, 2-tetrahydrofuranyl, 3-tetrahydrofuranyl, 2-tetrahydropyranyl, 3-tetrahydropyranyl, 4-tetrahydropyranyl, [1,3]-dioxalanyl, [1,3]-dithiolanyl, [1,3]-dioxanyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzo[1,3]dioxine, benzo[1,4]dioxine, benzopyrrolidinyl, benzopiperidinyl, benzoxolanyl, benzothiolanyl, 4,5,6,7-tetrahydropyrazol[1,5-a]pyridine, benzothianyl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-2-yl (C-attached).

A substituent is “substitutable” if it comprises at least one carbon or nitrogen atom that is bonded to one or more hydrogen atoms. Thus, for example, hydrogen, halogen, and cyano do not fall within this definition.

If a substituent is described as being “substituted,” a non-hydrogen substituent is in the place of a hydrogen substituent on a carbon or nitrogen of the substituent. Thus, for example, a substituted alkyl substituent is an alkyl substituent wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R″, wherein R′ and R″ together with the nitrogen atom to which they are attached may form a heterocyclic ring comprising 1 or 2 heteroatoms independently selected from oxygen, nitrogen, or sulfur, wherein said heterocycloalkyl moiety may be optionally substituted. The heterocyclic ring formed from R′ and R″ together with the nitrogen atom to which they are attached may be partially or fully saturated, or aromatic. In one embodiment, the heterocyclic ring consists of 4 to 10 atoms. In another embodiment, the heterocyclic ring is selected from the group consisting of piperidinyl, morpholinyl, azetidinyl, pyrrolyl, imidazolyl, pyrazolyl, triazolyl, tetrazolyl.

This specification uses the terms “substituent,” “radical,” and “group” interchangeably.

If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents.

If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non-hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen.

A prefix attached to a multi-moiety substituent only applies to the first moiety. To illustrate, the term “alkylcycloalkyl” contains two moieties: alkyl and cycloalkyl. Thus, a C₁₋₆- prefix on C₁₋₆alkylcycloalkyl means that the alkyl moiety of the alkylcycloalkyl contains from 1 to 6 carbon atoms; the C₁₋₆- prefix does not describe the cycloalkyl moiety. To illustrate further, the prefix “halo” on haloalkoxyalkyl indicates that only the alkoxy moiety of the alkoxyalkyl substituent is substituted with one or more halogen substituents. If the halogen substitution only occurs on the alkyl moiety, the substituent would be described as “alkoxyhaloalkyl.” If the halogen substitution occurs on both the alkyl moiety and the alkoxy moiety, the substituent would be described as “haloalkoxyhaloalkyl.”

If substituents are described as being “independently selected” from a group, each substituent is selected independent of the other(s). Each substituent therefore may be identical to or different from the other substituent(s).

As used herein the term “Formula I” may be hereinafter referred to as a “compound(s) of the invention.” Such terms are also defined to include all forms of the compound of Formula I, including hydrates, solvates, isomers, crystalline and non-crystalline forms, isomorphs, polymorphs, and metabolites thereof. For example, the compounds of Formula I, or pharmaceutically acceptable salts thereof, may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of Formula I may exist as clathrates or other complexes. Included within the scope of the invention are complexes such as clathrates, drug-host inclusion complexes wherein, in contrast to the aforementioned solvates, the drug and host are present in stoichiometric or non-stoichiometric amounts. Also included are complexes of Formula I containing two or more organic and/or inorganic components which may be in stoichiometric or non-stoichiometric amounts. The resulting complexes may be ionized, partially ionized, or non-ionized. For a review of such complexes, see J. Pharm. Sci., 64 (8), 1269-1288 by Haleblian (August 1975).

The compounds of Formula I may have asymmetric carbon atoms. The carbon-carbon bonds of the compounds of Formula I may be depicted herein using a solid line (-), a solid wedge ( [symbol] ), or a dotted wedge ( [symbol] ). The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of Formula I may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of Formula I can exist as enantiomers and diastereomers or as racemates and mixtures thereof. The use of a solid line to depict bonds to one or more asymmetric carbon atoms in a compound of Formula I and the use of a solid or dotted wedge to depict bonds to other asymmetric carbon atoms in the same compound is meant to indicate that a mixture of diastereomers is present.

Stereoisomers of Formula I include cis and trans isomers, optical isomers such as R and S enantiomers, diastereomers, geometric isomers, rotational isomers, conformational isomers, and tautomers of the compounds of Formula I, including compounds exhibiting more than one type of isomerism; and mixtures thereof (such as racemates and diastereomeric pairs). Also included are acid addition or base addition salts wherein the counterion is optically active, for example, D-lactate or L-lysine, or racemic, for example, DL-tartrate or DL-arginine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds of Formula I may exhibit the phenomena of tautomerism and structural isomerism. For example, the compounds of Formula I may exist in several tautomeric forms, including the enol and imine forms, and the keto and enamine forms, and geometric isomers and mixtures thereof. All such tautomeric forms are included within the scope of compounds of Formula I. Tautomers exist as mixtures of a tautomeric set in solution. In solid form, usually one tautomer predominates. Even though one tautomer may be described, the present invention includes all tautomers of the compounds of Formula I.

The present invention also includes isotopically-labeled compounds, which are identical to those recited in Formula I above, but for the fact that one or more atoms are replaced by an atom having an atomic mass or mass number different from the atomic mass or mass number usually found in nature. Examples of isotopes that may be incorporated into compounds of Formula I include isotopes of hydrogen, carbon, nitrogen, oxygen, phosphorus, fluorine and chlorine, such as, but not limited to, ²H, ³H, ¹¹C, ¹³C, ¹⁴C, ¹⁵N, ¹⁸O, ¹⁷O, ³²P, ³⁵S, ¹⁸F, and ³⁶C. Certain isotopically-labeled compounds of Formula I, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled compounds of Formula I may generally be prepared by carrying out the procedures disclosed in the Schemes and/or in the Examples and Preparations below, by substituting an isotopically-labeled reagent for a non-isotopically-labeled reagent.

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formula I with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention, which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include but are not limited to aliphatic, cycloaliphatic, aromatic, araliphatic, heterocyclic, carboxylic, and sulfonic classes of organic acids.

Specific examples of suitable organic acids include but are not limited to acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, and undecanoate.

Furthermore, where the compounds of the invention carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (i.e., dimethyl, diethyl, dibutyl, and diamyl sulfates), long chain halides (i.e., decyl, lauryl, myristyl, and stearyl chlorides, bromides, and iodides), arylalkyl halides (i.e., benzyl and phenethyl bromides), and others.

In one embodiment, hemisalts of acids and bases may also be formed, for example, hemisulphate and hemicalcium salts.

Typically, a compound of the invention is administered in an amount effective to treat a condition as described herein. The compounds of the invention are administered by any suitable route in the form of a pharmaceutical composition adapted to such a route, and in a dose effective for the treatment intended. Therapeutically effective doses of the compounds required to treat the progress of the medical condition are readily ascertained by one of ordinary skill in the art using preclinical and clinical approaches familiar to the medicinal arts. The term “therapeutically effective amount” as used herein refers to that amount of the compound being administered which will relieve to some extent one or more of the symptoms of the disorder being treated.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

The compounds of the invention may be administered orally. Oral administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions. In one embodiment, the total daily dose of a compound of the invention (administered in single or divided doses) is typically from about 0.01 to about 100 mg/kg. In another embodiment, the total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

In another embodiment, the invention comprises the use of one or more compounds of the invention for the preparation of a medicament for the treatment of the conditions recited herein.

For the treatment of the conditions referred to above, the compounds of the invention can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise a compound of the invention presented with a pharmaceutically acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of formula I are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (i.e., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using suitable dispersing, wetting, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, Finnin and Morgan, J. Pharm. Sci., 88 (10), 955-958 (1999).

Formulations suitable for topical administration to the eye include, for example, eye drops wherein the compound of this invention is dissolved or suspended in a suitable carrier. A typical formulation suitable for ocular or aural administration may be in the form of drops of a micronised suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed-linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone; as a mixture, for example, in a dry blend with lactose; or as a mixed component particle, for example, mixed with phospholipids, such as phosphatidylcholine) from a dry powder inhaler or as an aerosol spray from a pressurised container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman ef al., Eds., Pharmaceutical Dosage Forms, Marcel Decker, New York, N.Y., 1980; and Kibbe ef al., Eds., Handbook of Pharmaceutical Excipients (3^{rd} Ed.), American Pharmaceutical Association, Washington, 1999.

The compounds of the present invention can be used, alone or in combination with other therapeutic agents, in the treatment of various conditions or disease states. The compound(s) of the present invention and other therapeutic agent(s) may be administered simultaneously (either in the same dosage form or in separate dosage forms) or sequentially. An exemplary therapeutic agent may be, for example, a metabotropic glutamate receptor agonist.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

The present invention further comprises kits that are suitable for use in performing the methods of treatment described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

In another embodiment, the invention relates to the novel intermediates useful for preparing the compounds of the invention.

### General Synthetic Schemes

The compounds of the Formula I may be prepared by the methods described below, together with synthetic methods known in the art of organic chemistry, or modifications and transformations that are familiar to those of ordinary skill in the art. The starting materials used herein are commercially available or may be prepared by routine methods known in the art (such as those methods disclosed in standard reference books such as the COMPENDIUM OF ORGANIC SYNTHETIC METHODS, Vol. I-XII (published by Wiley-Interscience)). Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991; and T. W. Greene and P. G. M. Wuts, Protective Groups in Organic Chemistry, John Wiley & Sons, 1999, which are hereby incorporated by reference.

Compounds of Formula I, or their pharmaceutically acceptable salts, can be prepared according to the reaction Schemes discussed herein below. Unless otherwise indicated, the substituents in the Schemes are defined as above. Isolation and purification of the products is accomplished by standard procedures, which are known to a chemist of ordinary skill.

It will be understood by one skilled in the art that the various symbols, superscripts and subscripts used in the schemes, methods and examples are used for convenience of representation and/or to reflect the order in which they are introduced in the schemes, and are not intended to necessarily correspond to the symbols, superscripts or subscripts in the appended claims. The schemes are representative of methods useful in synthesizing the compounds of the present invention. They are not to constrain the scope of the invention in any way.

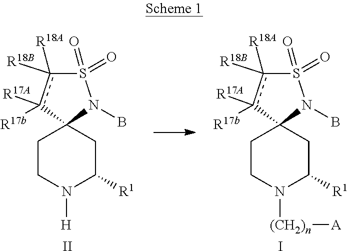

Scheme 1 refers to the preparation of compounds of the Formula I. Referring to Scheme 1, the compound of Formula I can be prepared from the compound of Formula II by reductive amination with an aldehyde under conditions well known to one of ordinary skill in the art, for instance by reaction with sodium triacetoxyborohydride, sodium cyanoborohydride or sodium borohydride in a solvent such as 1,2-dichloroethane, dichloromethane or alcohols such as methanol or ethanol. Preferably, the reaction is conducted with sodium triacetoxyborohydride in dichloroethane, to provide the compound of Formula I. Alternatively, alkylation of the compound of Formula II with a compound X—(CH₂)ₙ-A (X=Cl, Br, I), using a base such as cesium carbonate, potassium carbonate or sodium bicarbonate in a solvent such as acetonitrile, acetone or N,N-dimethylformamide (DMF), affords the compound of Formula I. Preferably the reaction is conducted in DMF using cesium carbonate as base.

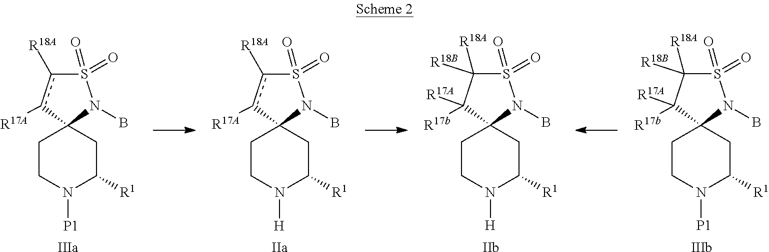

Scheme 2 refers to the preparation of compounds of Formula IIa and IIb. Compounds of Formula IIa and IIb can be converted into compounds of Formula I according to the methods of Scheme 1. Referring to Scheme 2, the compound of Formula IIIa, wherein P1 is a protecting group, can be deprotected by a variety of means well known to those skilled in the art to provide the compound of Formula IIa. The compound of Formula IIb wherein R^{17B} and R^{18B} are H can be prepared from the compound of Formula IIa via hydrogenation using standard methods, for example using a catalyst such as palladium on carbon in a solvent such as ethanol. An alternate preparation of the compound of Formula IIb, wherein R^{17B} and R^{18B} may or may not be H, is accomplished by deprotection of the compound of Formula IIIb. In the case where P1 is tert-butoxycarbonyl or benzyloxycarbonyl, IIIa and IIIb may be conveniently deprotected to afford, respectively, IIa and IIb by treatment with hydrogen bromide in acetic acid or water, or with aqueous hydrochloric acid.

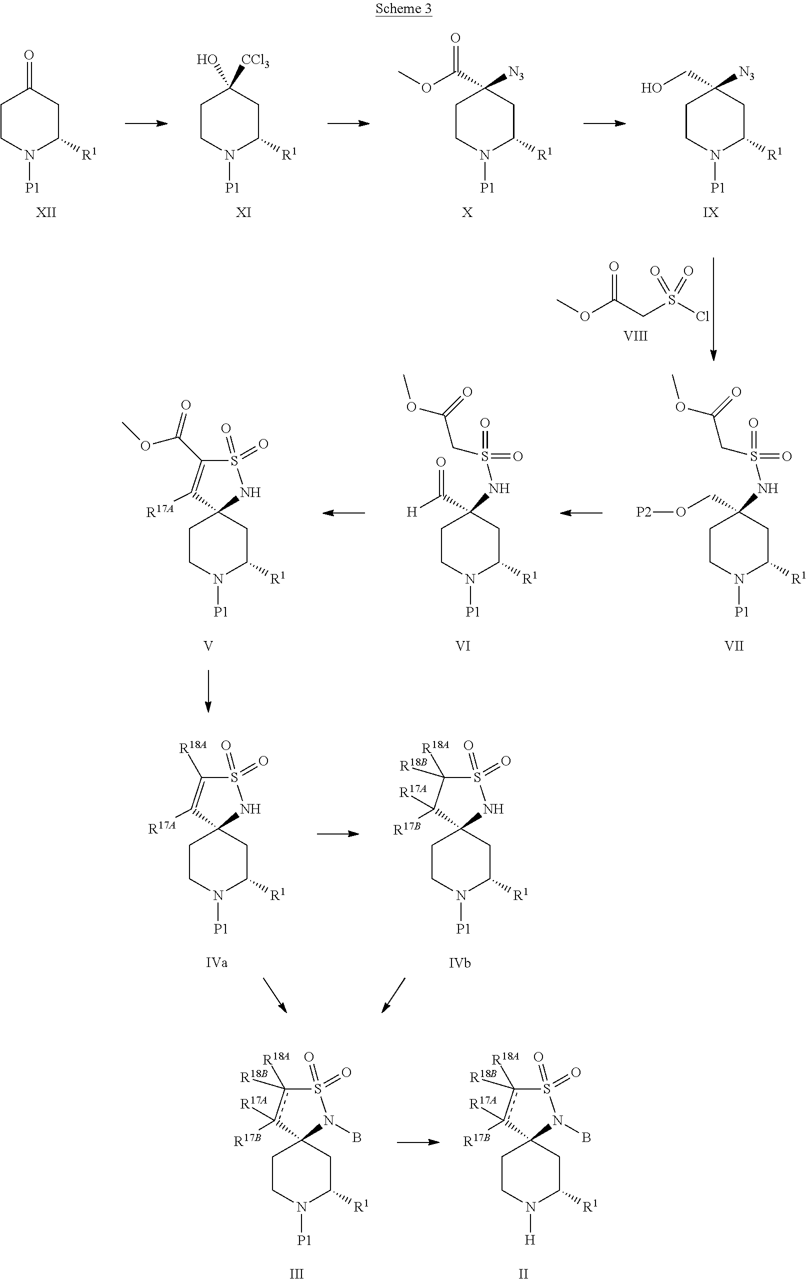

Scheme 3 illustrates an alternate preparation of compounds of the Formula II, wherein R^{17A}, R^{17B}, R^{18A} and R^{18B} are H, employing methods well known to one skilled in the art. Compounds of Formula II can be converted into compounds of Formula I according to the methods of Scheme 1. Referring to Scheme 3, base-mediated addition of chloroform to an appropriately protected chiral piperidinone of Formula XII (which may be prepared according to the method of S. Richards et al., Bioorg. Med. Chem. Lett. 2006, 16, 6241-6245, followed by chiral separation) provides the chiral compound of Formula XI after separation of diastereomers. Typical bases include lithium bis(trimethylsilyl)amide or lithium diisopropylamide in a solvent such as 1,2-dimethoxyethane or tetrahydrofuran. Reaction with sodium azide, under the influence of a base such as diazabicyclo[5.4.0]undec-7-ene, affords the azidoester of Formula X, which is then subjected to azide reduction, for instance with metallic zinc or tin, followed by ester reduction, with an agent such as sodium borohydride in alcoholic solvent, to afford the aminoalcohol of Formula IX. The free alcohol of Formula IX can be protected with a suitable silane, for instance through the action of tert-butyldimethylsilyl chloride in the presence of a base such as N,N-dimethylpyridin-4-amine; subsequent sulfonylation of the amine with an appropriate sulfonyl chloride derivative, for instance the compound of Formula VIII (prepared for example according to the method of J. B. Grimm ef al., J. Org. Chem. 2007, 72, 8135-8138), yields a compound of Formula VII. Deprotection of the alcohol with fluoride ion, followed by oxidation to the aldehyde, for instance with Dess-Martin periodinane or a Swern oxidation, affords the compound of Formula VI. Ring closure to the ester sultam of Formula V can be effected using piperidine, followed by decarboxylation via the Krapcho protocol to provide the N-protected sultam of Formula IVa. The compound of Formula IVb can be prepared from the compound of Formula IVa via hydrogenation, for example using a catalyst such as palladium on carbon in a solvent such as ethanol. Compounds of Formula III may be prepared from sulfonamides of Formula IVa and IVb by employing methods well known to one skilled in the art. Aryl or heteroaryl functionality may be added through addition of an activated aromatic such as 2-bromo-6-methylpyridine via palladium-catalyzed reaction mediated by a ligand such as, but not limited to, 5-(di-tert-butylphosphino)-1′,3′,5′-triphenyl-1′H-1,4′-bipyrazole together with a suitable base at elevated temperature. Another method of introducing aryl or heteroaryl functionality involves reaction of IVa or IVb with a bromoaryl or bromoheteroaryl moiety in the presence of a palladium catalyst such as tris(dibenzylideneacetone)dipalladium(0) and xantphos (4,5-bis(diphenylphosphino)-9,9-dimethylxanthene). The presence of a base, for instance cesium carbonate or potassium phosphate, is advantageous; an inert solvent, such as 1,4-dioxane, is preferred. The reaction can be carried out with conventional heating or in a microwave. Suitable reaction temperatures can range from about 25° C. to about 180° C., preferably from about 40° C. to about 110° C. with conventional heating, and from about 100° C. to 170° C. in a microwave reactor. The reaction is complete within about 10 minutes to about 4 hours in the microwave, and within from about 2 hours to about 48 hours with conventional heating. Aryl or heteroaryl groups may also be introduced via copper(I) iodide-mediated reaction of IVa or IVb with aryl or heteroaryl halides, using procedures described in A. Klapars et al., J. Am. Chem. Soc. 2001, 123, 7727-7729. Alternatively, alkylation of compounds of Formula IVa or IVb may be carried out using the appropriate reactant X—B (X=F, Cl, Br, I) and a base such as sodium hydride or cesium carbonate in DMF, or via a Mitsunobu reaction with the appropriate reactant B—OH to yield additional compounds of Formula III. Compounds of Formula II can then be prepared by deprotection of the amino group of Formula III.

Scheme 4 depicts an alternate preparation of the compound of Formula IIIa, wherein R^{18A} is H, employing methods well known to one skilled in the art. Compounds of Formula IIIa can be converted into compounds of Formula I according to the methods of Schemes 2 and 1. Referring to Scheme 4, Strecker reaction of an appropriately protected chiral piperidinone of the Formula XII with an aniline or aminoheterocycle and zinc cyanide in acetic acid, followed by diastereomer separation, provides the chiral compound of Formula XVII. Acylation of the amine of Formula XVII with an appropriate sulfonyl chloride and base, followed by further reaction with a base such as an alkali metal alkoxide, for example sodium methoxide in an alcohol solvent, affords the compound of Formula XVI. Decarboxylation can then be accomplished by ester hydrolysis with aqueous base under heating to provide the compound of Formula XV, wherein R^{18A} is H. Formation of the keto-sulfonamide of Formula XIV is accomplished by hydrolysis with aqueous acid. Reduction of the carbonyl group of Formula XIV, for example with sodium borohydride, affords the alcohol of Formula XIII where R^{17A}=H. Compounds wherein R^{17A} does not equal H may be prepared by reaction of the ketone of Formula XIV with a reagent such as R^{17A}—Li or R^{17A}—MgBr. Conversion of the alcohol of Formula XIII to the mesylate and elimination, mediated by a base such as 1,8-diazabicyclo[5.4.0]undec-7-ene, provides the compound of Formula IIIa, wherein R^{18A} is H.

The compound of Formula XIV can also be used to prepare compounds wherein R^{17B} is hydroxyl, (C₁₋₆alkyl)-O— or substituted amino, through functional group manipulations familiar to those skilled in the art. For example, reduction of the keto group of Formula XIV, for instance with sodium borohydride, affords the alcohol of Formula XIII, which can be alkylated using an alkyl halide and base to provide ethers of Formula IIIc. Alternatively, the compound of Formula XIV can be converted to compounds of Formula I wherein a double bond is present between groups R^{17A} and R^{18A}, and R^{17A} is a substituted amine or an alkoxy group: reaction of the compound of Formula XIV with an amine and acetic acid, or with, for example, dimethyl sulfate, provides compounds of the Formula IIId. Compounds of Formulas IIIc and IIId may be converted to compounds of Formula I according to the methods of Schemes 2 and 1.

The compound of Formula XIV can also be used to prepare compounds IIIa wherein R^{17A} is H and R^{18A} is an alkyl group or a substituted alkyl, aryl or heteroaryl group. Those skilled in the art will recognize that the activated methylene of Formula XIV (R^{18A} and R^{18B}=H) may be treated with a suitable base and reacted with an aryl, alkyl or heteroaryl halide, optionally in the presence of a transition metal catalyst, to form a compound of the Formula XIV wherein R^{18A} is optionally substituted aryl, alkyl or heteroaryl and R^{18B}=H; this compound may be converted to a compound of the Formula XIII and then dehydrated as described above to prepare a compound of Formula IIIa.

Another method for conversion of the compound of Formula XII to the compound of Formula IVa, wherein R^{18A} may be H or a group other than H, is shown in Scheme 5. Compounds of Formula IVa can be converted into compounds of Formula I according to the methods of Schemes 3, 2 and 1. Referring to Scheme 5, the ketone of Formula XII can be olefinated via a Horner-Emmons reaction employing methyl (dimethoxyphosphoryl)acetate and base, followed by reduction of the resulting ester moiety with a hydride reagent such as diisobutylaluminum hydride or lithium triethylborohydride, to afford the compound of Formula XXI as a mixture of olefin isomers. Subjection of the alcohol of Formula XXI to reaction with trichloroacetonitrile provides an intermediate imidate, which can be induced to rearrange via extended exposure to heat, to provide the trichloroacetamide of Formula XX. Removal of the trichloroacetamide group, for example by reduction of the amide with diisobutylaluminum hydride, followed by base-mediated sulfonylation of the resulting amine with the requisite vinyl sulfonyl reagent provides the divinyl compound of Formula XIX. Cyclization to the compound of Formula IVa can then be carried out via a metathesis reaction, for example using the Grubbs second generation catalyst 1,3-bis-(2,4,6-trimethylphenyl)-2-imidazolidinylidene)dichloro(phenylmethylene)-(tricyclohexylphosphine)ruthenium.

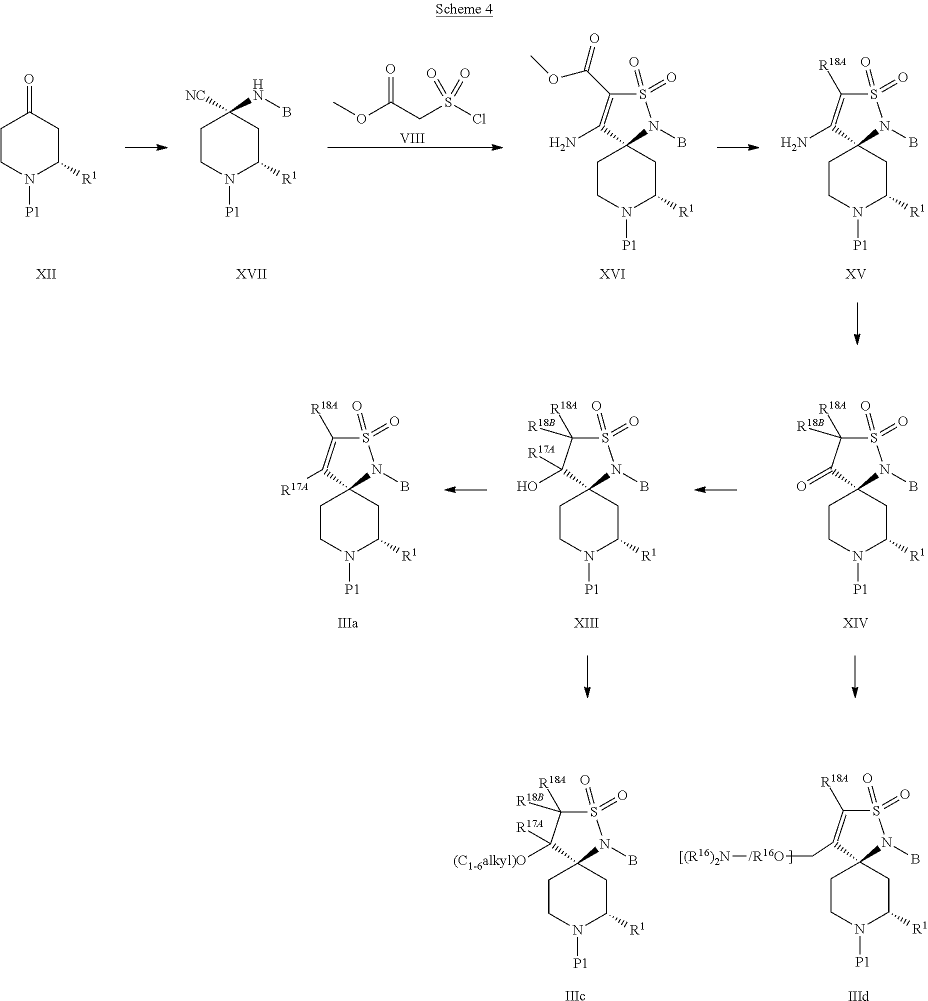

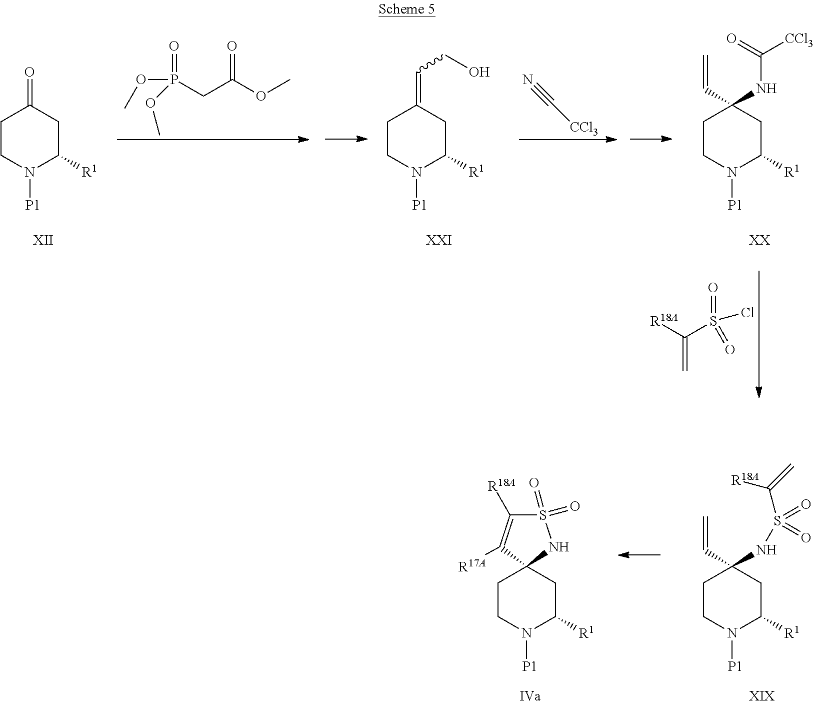

Referring to Scheme 6, compounds of Formula I wherein R^{18A} and/or R^{18B} are not hydrogen may also be prepared via mono- or bis-alkylation of the compound of Formula XIVc, after deprotonation with a base such as lithium diisopropylamide. The resulting compound of Formula XIV may be converted into a compound of Formula I according to the methods of Schemes 4, 2 and 1.

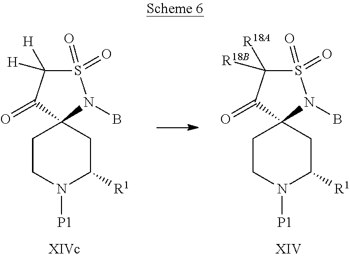

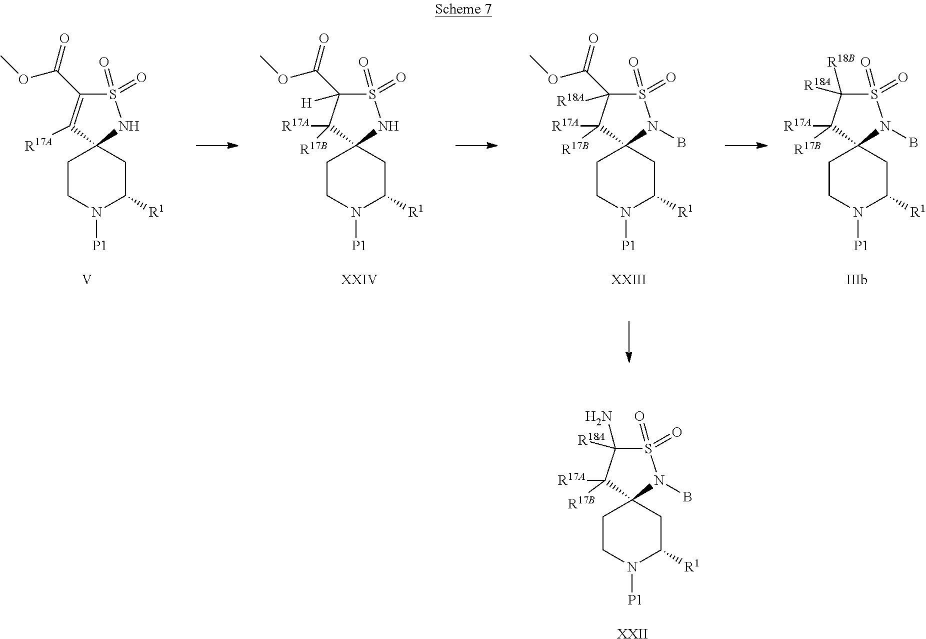

Various R^{18A} and R^{18B} groups may also be introduced using the compound of Formula V. Referring to Scheme 7, hydrogenation of the olefin of Formula V provides the compound of Formula XXIV wherein R^{17B} is H. Introduction of moiety B onto the sultam nitrogen, according to the method of Scheme 3, can be followed by deprotonation adjacent to the ester group of the compound of Formula XXIII wherein R^{18A} is H and subsequent reaction with an appropriate reactant of Formula R^{18A}—X (X=Cl, Br, I). Hydrolysis of the ester group and decarboxylation of the resulting carboxylic acid provides a compound of Formula I Mb, wherein R^{17B} and R^{18B} are H. Conversion of the ester of Formula XXIII into numerous functional groups can be carried out by methods well known to those of ordinary skill in the art. For instance, hydrolysis to the corresponding carboxylic acid, followed by Curtius rearrangement, affords the amine of Formula XXII, which can be alkylated or subjected to reductive amination to provide further compounds of Formula I, according to the methods of Schemes 2 and 1.

Removal of the piperidine nitrogen protecting group (in the case where P1=benzyloxycarbonyl) is accomplished with hydrogenation over a suitable palladium catalyst, or through reaction with nucleophilic agents such as trimethylsilyl iodide or through the action of aqueous acid (such as 6 N HCl). In cases where P1=tert-butyloxycarbonyl, deprotection may be accomplished through the agency of acid in either aqueous or anhydrous solvents.

### Experimental Procedures and Working Examples

The following illustrate the synthesis of various compounds of the present invention. Additional compounds within the scope of this invention may be prepared using the methods illustrated in these Examples, either alone or in combination with techniques generally known in the art.

It will be understood that the intermediate compounds of the invention depicted above are not limited to the particular enantiomer shown, but also include all stereoisomers and mixtures thereof. It will also be understood that compounds of Formula I can include intermediates of compounds of Formula I. Experiments were generally carried out under inert atmosphere (nitrogen or argon), particularly in cases where oxygen- or moisture-sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Mass spectrometry data is reported from either liquid chromatography-mass spectrometry (LCMS), atmospheric pressure chemical ionization (APCI) or gas chromatography-mass spectrometry (GCMS) instrumentation. Chemical shifts for nuclear magnetic resonance (NMR) data are expressed in parts per million (ppm, δ) referenced to residual peaks from the deuterated solvents employed.

For syntheses referencing procedures in other Examples or Methods, reaction conditions (length of reaction and temperature) may vary. In general, reactions were followed by thin layer chromatography or mass spectrometry, and subjected to work-up when appropriate. Purifications may vary between experiments: in general, solvents and the solvent ratios used for eluants/gradients were chosen to provide appropriate RfS or retention times.

### Preparations

### Preparation 1: (5R,7S)-(3-Fluorophenyl)-7-methyl-2-thia-1,8-diazaspiro[4.5]dec-3-ene 2,2-dioxide (P1)

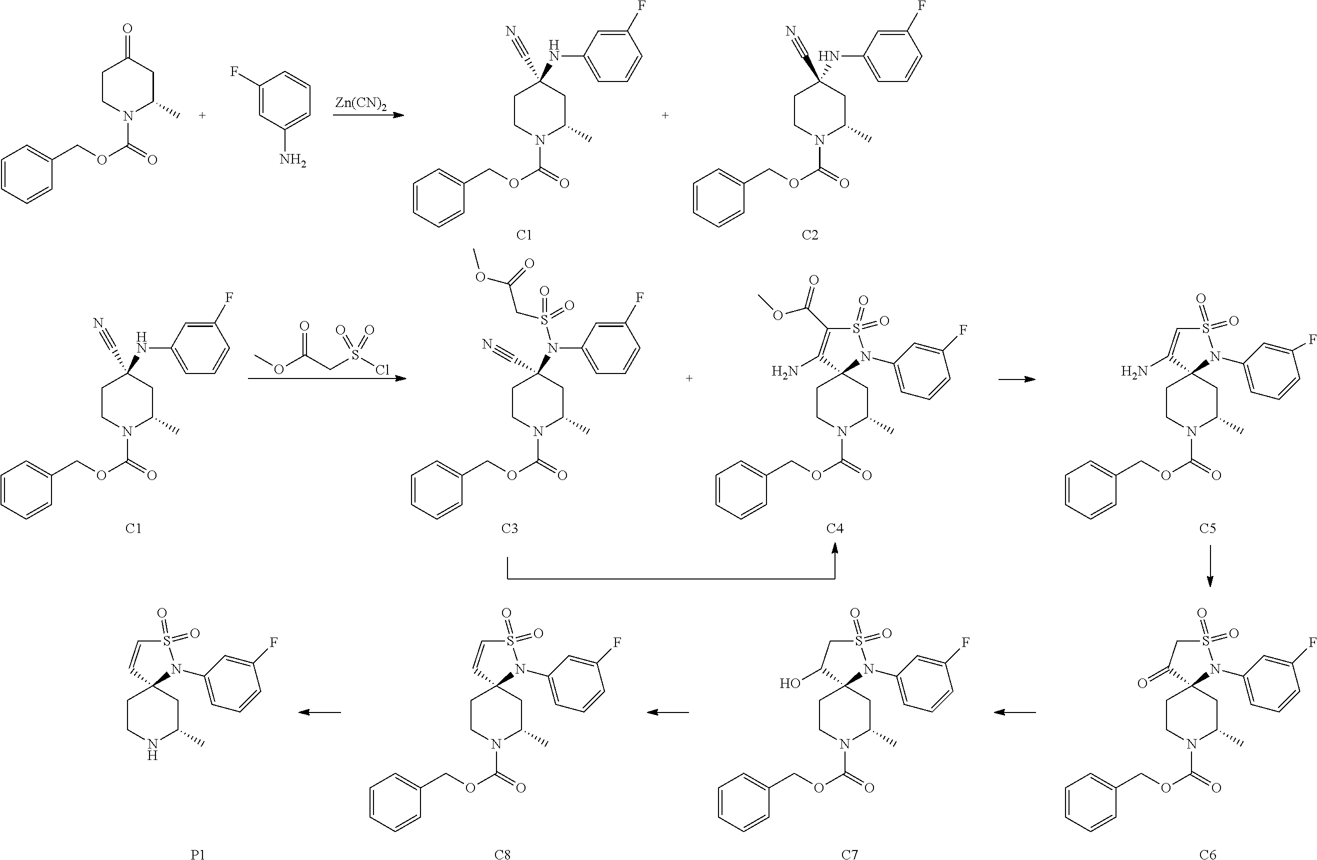

### Step 1. Synthesis of benzyl (2S,4R)-4-cyano-4-[(3-fluorophenyl)amino]-2-methylpiperidine-1-carboxylate (C1)

A solution of benzyl (2S)-2-methyl-4-oxopiperidine-1-carboxylate (see C. Coburn et. al., PCT Patent Application Publication WO 2007011810 A1 20070125) (31 g, 125 mmol) in acetic acid (250 mL) was treated with 3-fluoroaniline (24.1 mL, 250 mmol) followed by zinc cyanide (36.8 g, 313 mmol). The reaction mixture was allowed to stir at room temperature for 18 hours, at which time it was cooled in an ice bath and slowly basified with aqueous ammonium hydroxide solution. The resulting mixture was extracted three times with dichloromethane, and the combined organic layers were dried over sodium sulfate, filtered, and concentrated in vacuo. Purification of the residue by silica gel chromatography (Eluant: 20% to 40% ethyl acetate in heptane) afforded a mixture of C1 and its isomer benzyl (2S,4S)-4-cyano-4-[(3-fluorophenyl)amino]-2-methylpiperidine-1-carboxylate (C2) as an oil. Yield: 38 g, 103 mmol, 82%. This material was subjected to chromatography using a Chiralcel OJ-H column, 5 μm, 30×250 mm (Mobile phase: 80/20 CO₂/methanol; Flow rate: 80 g/min) to afford 16.5 g (36%) of C1 as an oil. MS (APCI) m/z 341.1 (M-CN)₊. ¹H NMR (400 MHz, CDCl₃) δ 7.32-7.41 (m, 5H), 7.18-7.24 (m, 1H), 6.60-6.68 (m, 3H), 5.16 (AB quartet, J_{AB}=12.3 Hz, Δν_{AB}=6.8 Hz, 2H), 4.59-4.67 (m, 1H), 4.24-4.31 (m, 1H), 3.74 (br s, 1H), 3.35 (ddd, J=14.6, 13.0, 2.4 Hz, 1H), 2.42-2.49 (m, 2H), 1.89 (dd, J=13.9, 6.5 Hz, 1H), 1.70 (ddd, J=13.1, 13.1, 4.4 Hz, 1H), 1.49 (d, J=7.2 Hz, 3H). The absolute configuration of this material was assigned based on X-ray crystallographic analysis of a single crystal of its isomer C2.

### X-Ray Analysis of Compound C2

A representative crystal was surveyed and a 0.85 Å data set (maximum sin Θ/λ=0.59) was collected on a Bruker APEX diffractometer. The absolute configuration was established through the known chiral center bearing the methyl group. Atomic scattering factors were taken from the International Tables for Crystallographyl¹ All crystallographic calculations were facilitated by the SHELXTL² system. All diffractometer data were collected at room temperature. Pertinent crystal, data collection, and refinement are summarized in Table 1.

A trial structure was obtained by direct methods. This trial structure refined routinely. Hydrogen positions were calculated wherever possible. The hydrogen on nitrogen was located by difference Fourier techniques and allowed to refine freely. The remaining hydrogen atoms were placed in idealized locations. The hydrogen parameters were added to the structure factor calculations but were not refined. The shifts calculated in the final cycles of least squares refinement were all less than 0.1 of the corresponding standard deviations. The final R-index was 3.11%. A final difference Fourier revealed no missing or misplaced electron density.

Coordinates, anisotropic temperature factors, distances and angles are given in Tables 2-5.

### REFERENCES FOR X-RAY CRYSTALLOGRAPHY STUDY

1. International Tables for Crystallography, Vol. C, pp. 219, 500, Kluwer Academic Publishers, 1992.
2. SHELXTL, Version 5.1, Bruker AXS, 1997.
3. H. D. Flack. Acta Crystalloqr. A39. 876-881, 1983.

TABLE 1
Crystal data and structure refinement for (2S,4S)-4-cyano-4-[(3-
fluorophenyl)amino]-2-methylpiperidine-1-carboxylate (C2)

| Empirical formula | C₂₁ H₂₂ N₃O₂ F | |
|---|---|---|
| Formula weight | 367.42 | |
| Temperature | 298(2) K | |
| Wavelength | 1.54178 Å | |
| Crystal system | Orthorhombic | |
| Space group | P2(1)2(1)2(1) | |
| Unit cell dimensions | a = 7.20870(10) Å | α = 90°. |
| | b = 12.5094(2) Å | β = 90°. |
| | c = 21.4005(3) Å | γ = 90°. |

| | |
|---|---|
| Volume | 1929.82(5) Å³ |
| Z | 4 |
| Density (calculated) | 1.265 Mg/m³ |
| Absorption coefficient | 0.731 mm⁻¹ |
| F(000) | 776 |
| Crystal size | 0.24 × 0.28 × 0.66 mm³ |
| Theta range for data collection | 4.09 to 64.96°. |
| Index ranges | −8 <= h <= 7, −14 <= k <= |
| | 14, −25 <= l <= 22 |
| Reflections collected | 10323 |
| Independent reflections | 2930 [R(int) = 0.0309] |
| Completeness to theta = 64.96° | 91.3% |
| Absorption correction | Empirical Absorption Correction |
| Refinement method | Full-matrix least-squares on F² |
| Data/restraints/parameters | 2930/0/265 |
| Goodness-of-fit on F² | 1.024 |
| Final R indices [I > 2sigma(I)] | R1 = 0.0311, wR2 = 0.0842 |
| R indices (all data) | R1 = 0.0326, wR2 = 0.0858 |
| Absolute structure parameter | 0.07(18) |
| Extinction coefficient | 0.0268(10) |
| Largest diff. peak and hole | 0.117 and −0.135 e.Å⁻³ |

TABLE 2
Atomic coordinates (×10⁴) and equivalent isotropic displacement
parameters (Å² × 10³) for (2S,4S)-4-cyano-4-[(3-fluorophenyl)amino]-2-
methylpiperidine-1-carboxylate (C2). U(eq) is defined as one third of
the trace of the orthogonalized Uᵢⱼ tensor.

| | x | y | z | U(eq) |
|---|---|---|---|---|
| C(1) | 11817(2) | 9652(1) | 9977(1) | 50(1) |
| C(2) | 12358(3) | 10369(2) | 10422(1) | 59(1) |
| C(3) | 11846(3) | 11424(2) | 10415(1) | 64(1) |
| C(4) | 10774(3) | 11759(1) | 9920(1) | 67(1) |
| C(5) | 10219(3) | 11065(1) | 9451(1) | 58(1) |
| C(6) | 10720(2) | 9992(1) | 9478(1) | 45(1) |
| N(7) | 10213(2) | 9221(1) | 9041(1) | 51(1) |
| C(8) | 8954(2) | 9349(1) | 8521(1) | 47(1) |
| C(9) | 8811(2) | 8266(1) | 8181(1) | 49(1) |
| C(10) | 7829(2) | 7433(1) | 8575(1) | 49(1) |
| N(11) | 5993(2) | 7816(1) | 8760(1) | 45(1) |
| C(12) | 5893(2) | 8853(1) | 9076(1) | 46(1) |
| C(13) | 6971(2) | 9686(1) | 8707(1) | 48(1) |
| C(14) | 4468(2) | 7197(1) | 8758(1) | 42(1) |
| O(15) | 4804(2) | 6214(1) | 8535(1) | 51(1) |
| C(16) | 3263(3) | 5492(1) | 8525(1) | 61(1) |
| C(17) | 3948(2) | 4418(1) | 8325(1) | 50(1) |
| C(18) | 5482(3) | 4296(1) | 7948(1) | 59(1) |
| C(19) | 6070(4) | 3282(2) | 7777(1) | 73(1) |
| C(20) | 5142(4) | 2391(1) | 7971(1) | 77(1) |
| C(21) | 3625(4) | 2506(2) | 8343(1) | 78(1) |
| C(22) | 3020(3) | 3509(2) | 8522(1) | 66(1) |
| O(23) | 2933(2) | 7473(1) | 8938(1) | 57(1) |
| C(24) | 6456(3) | 8757(2) | 9761(1) | 61(1) |
| C(25) | 9697(3) | 10143(1) | 8061(1) | 61(1) |
| N(26) | 10241(4) | 10743(2) | 7709(1) | 94(1) |
| F(27) | 13494(3) | 10044(1) | 10879(1) | 75(1) |
| F(27A) | 10716(11) | 12739(4) | 9878(4) | 92(3) |

TABLE 3
Bond lengths [Å] and angles [°] for (2S,4S)-4-cyano-
4-[(3-fluorophenyl)amino]-2-methylpiperidine-
1-carboxylate (C2)
Symmetry transformations used to generate equivalent atoms:

| | |
|---|---|
| C(1)—C(2) | 1.365(3) |
| C(1)—C(6) | 1.397(2) |
| C(2)—F(27) | 1.338(3) |
| C(2)—C(3) | 1.371(3) |
| C(3)—C(4) | 1.377(3) |
| C(4)—F(27A) | 1.230(6) |
| C(4)—C(5) | 1.385(3) |
| C(5)—C(6) | 1.391(2) |
| C(6)—N(7) | 1.393(2) |
| N(7)—C(8) | 1.445(2) |
| C(8)—C(25) | 1.497(2) |
| C(8)—C(9) | 1.541(2) |
| C(8)—C(13) | 1.542(2) |
| C(9)—C(10) | 1.516(2) |
| C(10)—N(11) | 1.463(2) |
| N(11)—C(14) | 1.345(2) |
| N(11)—C(12) | 1.4644(19) |
| C(12)—C(13) | 1.521(2) |
| C(12)—C(24) | 1.525(2) |
| C(14)—O(23) | 1.2207(19) |
| C(14)—O(15) | 1.3418(17) |
| O(15)—C(16) | 1.432(2) |
| C(16)—C(17) | 1.495(2) |
| C(17)—C(18) | 1.377(3) |
| C(17)—C(22) | 1.384(2) |
| C(18)—C(19) | 1.386(2) |
| C(19)—C(20) | 1.364(3) |
| C(20)—C(21) | 1.361(4) |
| C(21)—C(22) | 1.382(3) |
| C(25)—N(26) | 1.134(2) |
| C(2)—C(1)—C(6) | 119.66(16) |
| F(27)—C(2)—C(1) | 118.99(19) |
| F(27)—C(2)—C(3) | 117.72(18) |
| C(1)—C(2)—C(3) | 123.25(19) |
| C(2)—C(3)—C(4) | 116.92(17) |
| F(27A)—C(4)—C(3) | 112.3(4) |
| F(27A)—C(4)—C(5) | 124.2(4) |
| C(3)—C(4)—C(5) | 121.93(18) |
| C(4)—C(5)—C(6) | 119.96(18) |
| C(5)—C(6)—N(7) | 124.95(16) |
| C(5)—C(6)—C(1) | 118.24(16) |
| N(7)—C(6)—C(1) | 116.81(14) |
| C(6)—N(7)—C(8) | 127.20(13) |
| N(7)—C(8)—C(25) | 110.81(15) |
| N(7)—C(8)—C(9) | 107.87(13) |
| C(25)—C(8)—C(9) | 107.26(14) |
| N(7)—C(8)—C(13) | 114.41(14) |
| C(25)—C(8)—C(13) | 108.72(14) |
| C(9)—C(8)—C(13) | 107.47(13) |
| C(10)—C(9)—C(8) | 111.95(13) |
| N(11)—C(10)—C(9) | 110.37(13) |
| C(14)—N(11)—C(10) | 123.38(12) |
| C(14)—N(11)—C(12) | 118.12(13) |
| C(10)—N(11)—C(12) | 117.39(13) |
| N(11)—C(12)—C(13) | 109.96(13) |
| N(11)—C(12)—C(24) | 111.18(13) |
| C(13)—C(12)—C(24) | 114.65(14) |
| C(12)—C(13)—C(8) | 114.90(12) |
| O(23)—C(14)—O(15) | 122.34(14) |
| O(23)—C(14)—N(11) | 125.25(13) |
| O(15)—C(14)—N(11) | 112.41(13) |
| C(14)—O(15)—C(16) | 116.31(13) |
| O(15)—C(16)—C(17) | 108.34(14) |
| C(18)—C(17)—C(22) | 118.39(17) |
| C(18)—C(17)—C(16) | 122.22(15) |
| C(22)—C(17)—C(16) | 119.39(17) |
| C(17)—C(18)—C(19) | 120.08(18) |
| C(20)—C(19)—C(18) | 121.2(2) |
| C(21)—C(20)—C(19) | 119.03(19) |
| C(20)—C(21)—C(22) | 120.8(2) |
| C(21)—C(22)—C(17) | 120.5(2) |
| N(26)—C(25)—C(8) | 179.2(2) |

TABLE 4
Anisotropic displacement parameters (Å² × 10³) for
(2S,4S)-4-cyano-4-[(3-fluorophenyl)amino]-2-methylpiperidine-
1-carboxylate (C2). The anisotropic displacement factor exponent
takes the form: −2π²[h²a*²U¹¹ + . . . + 2 h k a* b* U¹²]

| | U¹¹ | U²² | U³³ | U²³ | U¹³ | U¹² |
|---|---|---|---|---|---|---|
| C(1) | 42(1) | 53(1) | 55(1) | −3(1) | 2(1) | −1(1) |
| C(2) | 50(1) | 73(1) | 55(1) | −7(1) | 2(1) | −6(1) |
| C(3) | 64(1) | 64(1) | 65(1) | −20(1) | 6(1) | −12(1) |
| C(4) | 75(1) | 47(1) | 80(1) | −13(1) | 7(1) | −5(1) |
| C(5) | 60(1) | 45(1) | 68(1) | −4(1) | −4(1) | −1(1) |
| C(6) | 37(1) | 44(1) | 55(1) | −5(1) | 6(1) | −1(1) |
| N(7) | 45(1) | 46(1) | 62(1) | −11(1) | −7(1) | 9(1) |
| C(8) | 45(1) | 44(1) | 52(1) | 0(1) | 1(1) | −2(1) |
| C(9) | 40(1) | 55(1) | 52(1) | −11(1) | 7(1) | −3(1) |
| C(10) | 40(1) | 41(1) | 65(1) | −9(1) | 5(1) | 5(1) |
| N(11) | 38(1) | 39(1) | 59(1) | −5(1) | 7(1) | 4(1) |
| C(12) | 40(1) | 41(1) | 56(1) | −4(1) | 2(1) | 8(1) |
| C(13) | 46(1) | 39(1) | 59(1) | 1(1) | −6(1) | 4(1) |
| C(14) | 41(1) | 40(1) | 45(1) | 1(1) | 2(1) | 5(1) |
| O(15) | 44(1) | 41(1) | 69(1) | −9(1) | 6(1) | −3(1) |
| C(16) | 46(1) | 57(1) | 80(1) | −15(1) | 8(1) | −11(1) |
| C(17) | 52(1) | 48(1) | 51(1) | −6(1) | −2(1) | −10(1) |
| C(18) | 65(1) | 51(1) | 61(1) | −6(1) | 11(1) | −7(1) |
| C(19) | 81(2) | 66(1) | 72(1) | −15(1) | 13(1) | 6(1) |
| C(20) | 109(2) | 48(1) | 73(1) | −9(1) | −11(1) | 3(1) |
| C(21) | 105(2) | 48(1) | 81(1) | 5(1) | 1−0(1) | −21(1) |
| C(22) | 67(1) | 66(1) | 65(1) | −3(1) | 3(1) | −19(1) |
| O(23) | 41(1) | 52(1) | 79(1) | −7(1) | 10(1) | 5(1) |
| C(24) | 62(1) | 67(1) | 53(1) | −5(1) | 8(1) | 2(1) |
| C(25) | 59(1) | 62(1) | 62(1) | 1(1) | 4(1) | −15(1) |
| N(26) | 109(2) | 89(1) | 82(1) | 14(1) | 8(1) | −39(1) |
| F(27) | 75(1) | 89(1) | 60(1) | −4(1) | −18(1) | −2(1) |
| F(27A) | 102(6) | 58(3) | 116(6) | −21(3) | −9(4) | 8(3) |

TABLE 5
Hydrogen coordinates (×10⁴) and isotropic displacement
parameters (Å² × 10³) for (2S,4S)-4-cyano-4-
[(3-fluorophenyl)amino]-2-methylpiperidine-1-carboxylate (C2).

| | x | y | z | U(eq) |
|---|---|---|---|---|
| H(1) | 12190 | 8917 | 10007 | 80 |
| H(2A) | 13780(80) | 10510(40) | 10730(30) | 80 |
| H(3) | 12217 | 11907 | 10740 | 80 |
| H(4A) | 9910(90) | 12460(50) | 10010(30) | 80 |
| H(5) | 9488 | 11325 | 9108 | 80 |
| H(7X) | 10960(40) | 8674(18) | 9002(12) | 80 |
| H(9A) | 8148 | 8361 | 7796 | 80 |
| H(9B) | 10036 | 8017 | 8081 | 80 |
| H(10A) | 7705 | 6783 | 8340 | 80 |
| H(10B) | 8553 | 7282 | 8940 | 80 |
| H(12) | 4616 | 9069 | 9070 | 80 |
| H(13A) | 7045 | 10328 | 8951 | 80 |
| H(13B) | 6290 | 9851 | 8334 | 80 |
| H(16A) | 2335 | 5744 | 8239 | 80 |
| H(16B) | 2717 | 5446 | 8933 | 80 |
| H(18) | 6146 | 4914 | 7804 | 80 |
| H(19) | 7149 | 3205 | 7518 | 80 |
| H(20) | 5553 | 1693 | 7846 | 80 |
| H(21) | 2968 | 1884 | 8484 | 80 |
| H(22) | 1947 | 3577 | 8784 | 80 |
| H(24A) | 7699 | 8485 | 9787 | 80 |
| H(24B) | 5625 | 8278 | 9971 | 80 |
| H(24C) | 6399 | 9448 | 9955 | 80 |

### Step 2. Synthesis of benzyl (2S,4R)-4-cyano-4-{(3-fluorophenyl)[(2-methoxy-2-oxoethyl)sulfonyl]amino}-2-methylpiperidine-1-carboxylate (C3) and 8-benzyl 3-methyl (5R,7S)-4-amino-1-(3-fluorophenyl)-7-methyl-2-thia-1,8-diazaspiro[4.5]dec-3-ene-3,8-dicarboxylate 2,2-dioxide (C4)

2,6-Dimethylpyridine (99%, 3.84 mL, 32.6 mmol) was added to a solution of benzyl (2S,4R)-4-cyano-4-[(3-fluorophenyl)amino]-2-methylpiperidine-1-carboxylate (C1) (4.00 g, 10.9 mmol) in dichloromethane (40 mL). After 5 minutes, the reaction mixture was cooled to 0° C. and treated with methyl (chlorosulfonyl)acetate (prepared according to the method of J. B. Grimm et al., J. Org. Chem. 2007, 72, 8135-8138) (4.70 g, 27.2 mmol). The ice bath was removed, and the reaction was allowed to warm to room temperature; after 1 hour, it was heated to 40° C. for 18 hours. The reaction was then poured into aqueous sodium bicarbonate solution, and the mixture was extracted three times with ethyl acetate. The combined organic layers were washed with saturated aqueous sodium chloride solution, dried over sodium sulfate, filtered and concentrated in vacuo. Chromatography on silica gel (Eluant: 3:1-ethyl acetate: heptane) afforded C3 as a yellow foam (Yield: 1.58 g, 3.14 mmol, 29%) and C4 as a yellow solid (Yield: 2.70 g, 5.36 mmol, 49%). Physical data for C3: LCMS m/z 504.5 (M+1). ¹H NMR (400 MHz, CDCl₃) δ 7.45 (ddd, J=8.2, 8.2, 6.3 Hz, 1H), 7.29-7.39 (m, 5H), 7.15-7.26 (m, 3H), 5.08-5.15 (m, 2H), 4.53-4.61 (m, 1H), 4.16-4.24 (m, 1H), 4.14 (br s, 2H), 3.85 (s, 3H), 3.26-3.35 (m, 1H), 2.51-2.64 (m, 1H), 2.16-2.30 (m, 1H), 1.92-1.99 (m, 1H), 1.46-1.56 (m, 1H), 1.44 and 1.45 (2 d, J=7.3 and 7.4 Hz, 3H). Physical data for C4: LCMS m/z 504.5 (M+1). ¹H NMR (400 MHz, CDCl₃) δ 7.22-7.39 (m, 7H), 7.19 (ddd, J=9.3, 2.2, 2.2 Hz, 1H), 7.06 (dddd, J=8.1, 8.1, 2.5, 1.3 Hz, 1H), 4.85 (AB quartet, J_{AB}=12.3 Hz, Δν_{AB}=67.3 Hz, 2H), 3.88 (s, 3H), 3.81-3.88 (m, 1H), 3.63-3.72 (m, 1H), 2.96 (ddd, J=14.6, 11.9, 5.1 Hz, 1H), 2.48 (ddd, J=16, 12, 7 Hz, 1H), 2.31 (dd, J=15, 6 Hz, 1H), 2.12 (ddd, J=16, 5, 2 Hz, 1H), 1.85 (dd, J=15.0, 11.1 Hz, 1H), 1.05 (d, J=6.2 Hz, 3H).

### Step 3. Conversion of benzyl (2S,4R)-4-cyano-4-{(3-fluorophenyl)[(2-methoxy-2-oxoethyl)sulfonyl]-amino}-2-methylpiperidine-1-carboxylate (C3) to 8-benzyl 3-methyl (5R,7S)-4-amino-1-(3-fluorophenyl)-7-methyl-2-thia-1,8-diazaspiro[4.5]dec-3-ene-3.8-dicarboxylate 2,2-dioxide (C4)

Sodium methoxide (95%, 312 mg, 5.48 mmol) was added to a solution of benzyl (2S,4R)-4-cyano-4-{(3-fluorophenyl)[(2-methoxy-2-oxoethyl)sulfonyl]amino}-2-methylpiperidine-1-carboxylate (C3) (1.38 g, 2.74 mmol) in methanol (14 mL), and the reaction was stirred at room temperature for 18 hours. It was then poured into an aqueous solution of sodium bicarbonate, and the mixture was extracted three times with ethyl acetate. The combined organic layers were washed with saturated aqueous sodium chloride solution, dried over sodium sulfate, filtered and concentrated in vacuo. Purification of the residue via silica gel chromatography (Gradient: 50% to 75% ethyl acetate in heptanes) provided the title product as an off-white solid. Yield: 446 mg, 0.886 mmol, 32%. LCMS m/z 502.7 (M−1). ¹H NMR (400 MHz, CDCl₃) δ 7.23-7.39 (m, 7H), 7.19 (ddd, J=9.3, 2.2, 2.2 Hz, 1H), 7.05-7.10 (m, 1H), 4.87 (AB quartet, J_{AB}=12.3 Hz, Δν_{AB}=63.3 Hz, 2H), 3.89 (s, 3H), 3.84-3.91 (m, 1H), 3.65-3.74 (m, 1H), 2.95 (ddd, J=14.4, 11.8, 5.0 Hz, 1H), 2.49 (br ddd, J=16, 12, 7 Hz, 1H), 2.34 (dd, J=15.0, 6.3 Hz, 1H), 2.12 (ddd, J=15.6, 4.8, 2.3 Hz, 1H), 1.82 (dd, J=15.0, 11.1 Hz, 1H), 1.07 (d, J=6.2 Hz, 3H).

### Step 4. Synthesis of benzyl (5R,7S)-4-amino-1-(3-fluorophenyl)-7-methyl-2-thia-1,8-diazaspiro[4.5]dec-3-ene-8-carboxylate 2,2-dioxide (C5)

An aqueous solution of lithium hydroxide (2.3 M, 5.18 mL, 11.9 mmol) was added to a solution of 8-benzyl 3-methyl (5R,7S)-4-amino-1-(3-fluorophenyl)-7-methyl-2-thia-1,8-diazaspiro[4.5]dec-3-ene-3,8-dicarboxylate 2,2-dioxide (C4) (600 mg, 1.19 mmol) in tetrahydrofuran (6 mL), and the mixture was heated at reflux for 18 hours. The reaction was then diluted with water and extracted three times with ethyl acetate. The combined organic layers were washed with saturated aqueous sodium chloride solution, dried over sodium sulfate, filtered and concentrated in vacuo. Purification via silica gel chromatography (Gradient: 50% to 100% ethyl acetate in heptane) afforded the title product as a white solid. Yield: 353 mg, 0.792 mmol, 67%. LCMS m/z 446.6 (M+1). ¹H NMR (400 MHz, CDCl₃) δ 7.23-7.39 (m, 8H), 7.05-7.10 (m, 1H), 5.63 (s, 1H), 4.86 (AB quartet, J_{AB}=12.5 Hz, Δν_{AB}=71 Hz, 2H), 4.19 (br s, 2H), 3.92-3.99 (m, 1H), 3.63-3.72 (m, 1H), 2.92 (ddd, J=14.3, 12.0, 4.7 Hz, 1H), 2.50 (ddd, J=16, 12, 6 Hz, 1H), 2.33 (dd, J=15, 6 Hz, 1H), 2.18 (ddd, J=15.6, 4.8, 2.4 Hz, 1H), 1.84 (dd, J=14.9, 11.1 Hz, 1H), 1.09 (d, J=6.2 Hz, 3H).

### Step 5. Synthesis of benzyl (5R,7S)-1-(3-fluorophenyl)-7-methyl-4-oxo-2-thia-1,8-diazaspiro[4.5]decane-8-carboxylate 2,2-dioxide (C6)

A solution of benzyl (5R,7S)-4-amino-1-(3-fluorophenyl)-7-methyl-2-thia-1,8-diazaspiro[4.5]dec-3-ene-8-carboxylate 2,2-dioxide (C5) (1.16 g, 2.60 mmol) in methanol (26 mL) was treated with aqueous hydrochloric acid (1 M, 20.8 mL, 20.8 mmol). Additional methanol (50 mL) was added, and the reaction mixture was stirred for 30 minutes. The reaction was basified with aqueous sodium bicarbonate solution, then extracted three times with ethyl acetate. The combined organic extracts were washed with saturated aqueous sodium chloride solution, dried over sodium sulfate, filtered and concentrated under reduced pressure. Silica gel chromatography of the residue (Eluant: dichloromethane) provided the product as a pale yellow foam. Yield: 1.05 g, 2.35 mmol, 90%. ¹H NMR (400 MHz, CDCl₃) δ 7.43 (ddd, J=8.1, 8.1, 6.3 Hz, 1H), 7.27-7.37 (m, 5H), 7.18-7.24 (m, 2H), 7.13 (ddd, J=9.1, 2.2, 2.2 Hz, 1H), 5.03 (AB quartet, J_{AB}=12.4 Hz, Δν_{AB}=16 Hz, 2H), 4.31-4.39 (m, 1H), 4.06 (AB quartet, J_{AB}=17.0 Hz, Δν_{AB}=22.3 Hz, 2H), 4.02-4.09 (m, 1H), 3.41-3.50 (m, 1H), 2.05-2.20 (m, 3H), 1.68-1.77 (m, 1H), 1.23 (d, J=7.0 Hz, 3H).

### Step 6. Synthesis of benzyl (5R,7S)-1-(3-fluorophenyl)-4-hydroxy-7-methyl-2-thia-1,8-diazaspiro[4.5]decane-8-carboxylate 2,2-dioxide (C7)

Sodium borohydride (88.6 mg, 2.34 mmol) was added to a suspension of benzyl (5R,7S)-1-(3-fluorophenyl)-7-methyl-4-oxo-2-thia-1,8-diazaspiro[4.5]decane-8-carboxylate 2,2-dioxide (C6) (950 mg, 2.13 mmol) in methanol (18 mL), and the reaction was allowed to proceed at room temperature for 1 hour. It was then poured into water, and the mixture was extracted three times with ethyl acetate. The combined organic layers were washed with saturated aqueous sodium chloride solution, dried over sodium sulfate, filtered and concentrated in vacuo to provide the product as a white foam. By ¹H NMR analysis this material was a mixture of alcohol diastereomers. Yield: 952 mg, 2.12 mmol, 99.5%. LCMS m/z 449.0 (M+1). ¹H NMR (400 MHz, CDCl₃), selected peaks, δ 7.38-7.45 (m, 1H), 7.28-7.37 (m, 5H), 7.16-7.22 (m, 1H), 7.08-7.12 (m, 1H), 7.02-7.07 (m, 1H), 5.03-5.11 (m, 2H), 3.64-3.72 (m, 1H), 3.44-3.51 (m, 1H), 3.20 and 3.32 (2 d, J=10.9 and 11.1 Hz, 1H), 1.27 and 1.30 (2 d, J=7.2 Hz, 3H).

### Step 7. Synthesis of benzyl (5R,7S)-1-(3-fluorophenyl)-7-methyl-2-thia-1,8-diazaspiro[4.5]dec-3-ene-8-carboxylate 2,2-dioxide (C8)

Methanesulfonyl chloride (0.215 mL, 2.77 mmol) was added to a 0° C. solution of benzyl (5R,7S)-1-(3-fluorophenyl)-4-hydroxy-7-methyl-2-thia-1,8-diazaspiro[4.5]decane-8-carboxylate 2,2-dioxide (C7) (952 mg, 2.12 mmol) and triethylamine (0.592 mL, 4.25 mmol) in dichloromethane (11 mL). After 1 hour, the reaction was poured into water and extracted three times with dichloromethane. The combined organic layers were washed with saturated aqueous sodium chloride solution, dried over sodium sulfate, filtered and concentrated to provide the intermediate mesylate. This material was dissolved in dichloromethane (10 mL), cooled to 0° C. and treated with 1,8-diazabicyclo[5.4.0]undec-7-ene (0.312 mL, 2.09 mmol). After 30 minutes, the reaction was poured into water and extracted three times with dichloromethane. The combined organic extracts were washed with saturated aqueous sodium chloride solution, dried over sodium sulfate, filtered and concentrated under reduced pressure. Silica gel chromatography (Gradient: 0% to 50% ethyl acetate in heptane) provided the product as a white foam. Yield: 713 mg, 1.66 mmol, 78%. LCMS m/z 431.6 (M+1). ¹H NMR (400 MHz, CDCl₃) δ 7.45 (ddd, J=8.1, 8.1, 6.4 Hz, 1H), 7.39 (d, J=7.4 Hz, 1H), 7.29-7.37 (m, 5H), 7.23 (dddd, J=8.3, 8.3, 2.5, 0.8 Hz, 1H), 7.16 (ddd, J=7.8, 1.8, 0.9 Hz, 1H), 7.10 (ddd, J=9.2, 2.2, 2.2 Hz, 1H), 6.91 (d, J=7.3 Hz, 1H), 5.04-5.11 (m, 2H), 4.57-4.66 (m, 1H), 4.16-4.26 (m, 1H), 3.08-3.17 (m, 1H), 2.06 (dd, J=13.7, 6.9 Hz, 1H), 1.74-1.86 (m, 3H), 1.29 (d, J=7.2 Hz, 3H).

### Step 8. Synthesis of (5R,7S)-(3-fluorophenyl)-7-methyl-2-thia-1,8-diazaspiro[4.5]dec-3-ene 2,2-dioxide (P1)

A suspension of benzyl (5R,7S)-1-(3-fluorophenyl)-7-methyl-2-thia-1,8-diazaspiro[4.5]dec-3-ene-8-carboxylate 2,2-dioxide (C8) (1.08 g, 2.51 mmol) in aqueous hydrochloric acid (6 M, 12.5 mL, 75 mmol) and 1,4-dioxane (5 mL) was heated to reflux for 3 hours. After cooling to room temperature, the reaction was poured into dichloromethane. The aqueous layer was basified with 1 N aqueous sodium hydroxide solution and extracted four times with dichloromethane; these organic layers were combined, dried over sodium sulfate, filtered and concentrated under reduced pressure to provide the product as a yellow solid. Yield: 636 mg, 2.15 mmol, 86%. ¹H NMR (400 MHz, CDCl₃) δ 7.40-7.47 (m, 1H), 7.27-7.30 (m, 1H), 7.18-7.24 (m, 2H), 6.76 (AB quartet, J_{AB}=7.0 Hz, Δν_{AB}=18.4 Hz, 2H), 2.86 (ddd, J=12.7, 5.0, 3.4 Hz, 1H), 2.60-2.68 (m, 1H), 2.56 (ddd, J=12.6, 11.9, 3.1 Hz, 1H), 2.05-2.13 (m, 2H), 1.92 (ddd, J=14.4, 11.8, 5.1 Hz, 1H), 1.57 (dd, J=14.2, 10.6 Hz, 1H), 1.00 (d, J=6.2 Hz, 3H).

### Alternate Preparation of benzyl (5R,7S)-7-methyl-2-thia-1,8-diazaspiro[4.5]dec-3-ene-8-carboxylate 2,2-dioxide core (C18)

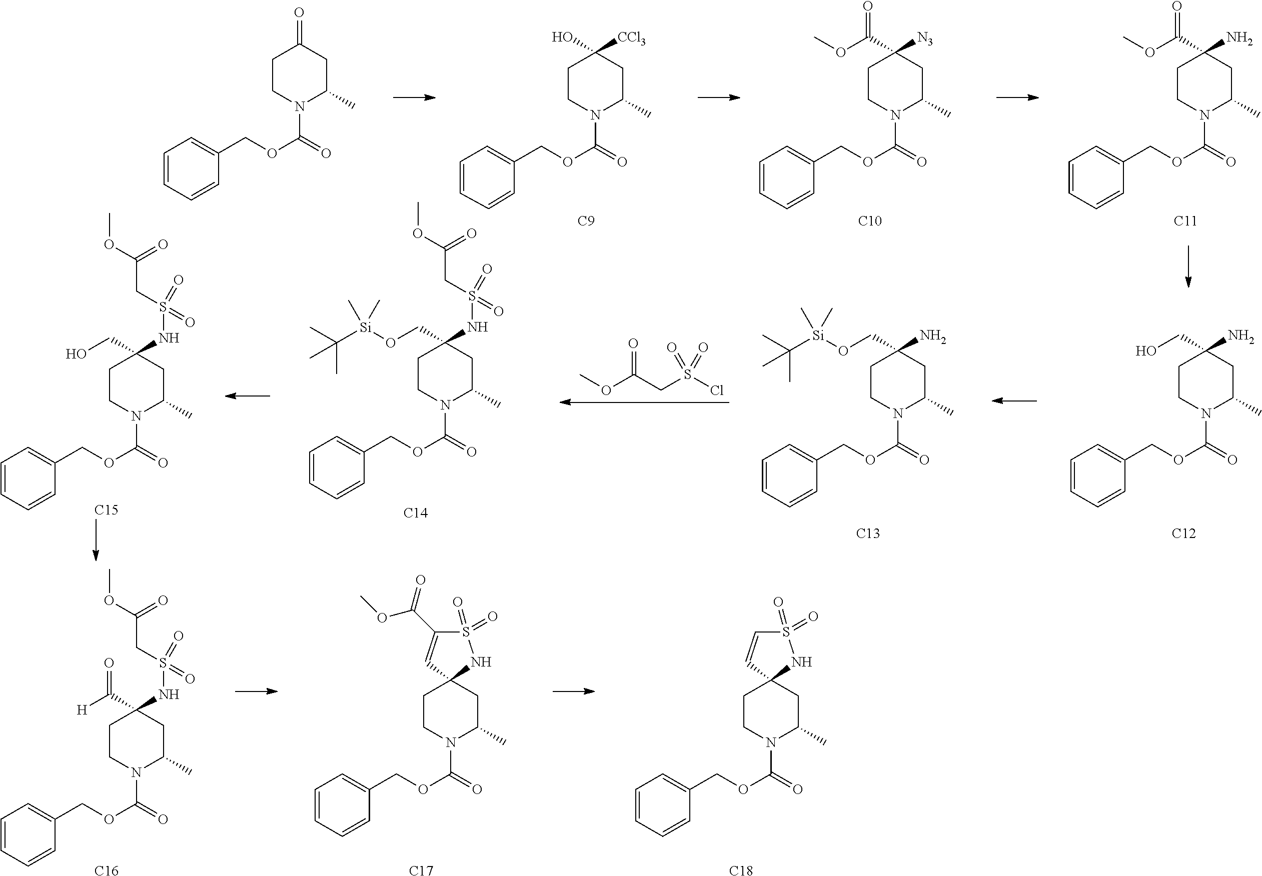

### Step 1. Synthesis of benzyl (2S,4S)-4-hydroxy-2-methyl-4-(trichloromethyl)piperidine-1-carboxylate (C9)

Chloroform (4.06 mL, 50.7 mmol) was added to a mixture of benzyl (2S)-2-methyl-4-oxopiperidine-1-carboxylate (98.5%, 4.24 g, 16.9 mmol) and magnesium chloride (4.83 g, 50.7 mmol) in 1,2-dimethoxyethane (45 mL), and the reaction mixture was cooled in a dry ice/acetone bath. Lithium bis(trimethylsilyl)amide (1 M in tetrahydrofuran, 25.4 mL, 25.4 mmol) was added drop-wise over 30 minutes, while keeping the internal temperature of the reaction below −72° C. The reaction was stirred at −72 to −77° C. for 4 hours, then allowed to warm to −15° C. by transferring the flask to a wet ice-methanol bath. After one hour at −15° C., the reaction was slowly quenched with water (25 mL), then partitioned between water (75 mL) and ethyl acetate (150 mL). The aqueous phase was extracted with ethyl acetate (2×50 mL), and the combined organic extracts were washed with saturated aqueous sodium chloride solution (75 mL), dried over magnesium sulfate, filtered and concentrated in vacuo. The crude product was dissolved in diethyl ether (30 mL), which caused a white precipitate to form; this mixture was stirred for 18 hours. The solid was collected by filtration and rinsed with cold diethyl ether (10 mL) to provide C9 as a white solid. Yield: 2.95 g, 8.05 mmol, 48%. ¹H NMR (400 MHz, DMSO-oV presumed to be a mixture of rotamers) δ 1.27 and 1.28 (2 d, J=6.9 Hz, 3H), 1.81-1.96 (m, 3H), 2.07-2.15 (m, 1H), 3.09-3.25 (m, 1H), 3.95-4.03 (m, 1H), 4.44-4.53 (m, 1H), 5.04-5.14 (m, 2H), 6.20 (s, 1H), 7.29-7.40 (m, 5H). The relative configuration of the methyl and hydroxy groups was determined by single-crystal X-ray crystallographic analysis of a sample prepared in an analogous manner; that sample was crystallized from acetonitrile-water.

### X-Ray Analysis of Compound C9

Data collection was performed on a Bruker APEX diffractometer at room temperature. Data collection consisted of 3 omega scans at low angle and three at high angle; each with 0.5 step. In addition, 2 phi scans were collected to improve the quality of the absorption correction.

The structure was solved by direct methods using SHELXTL software suite in the space group P2(1)2(1)2(1). The structure was subsequently refined by the full-matrix least squares method. All non-hydrogen atoms were found and refined using anisotropic displacement parameters.

All remaining hydrogen atoms were placed in calculated positions and were allowed to ride on their carrier atoms. The final refinement included isotropic displacement parameters for all hydrogen atoms. The hydrogen atom bonded to O3 was refined as a rotating OH (AFIX 147).

From this crystal structure it has been possible to assign the absolute configuration of the molecule directly from the x-ray diffraction data. The structure was refined as depicted with the flack parameter=0.023 with an esd of 0.019. Additionally, the Hooft parameter=0.033 with an esd of 0.012

Pertinent crystal, data collection and refinement are summarized in Table 6. Atomic coordinates, bond lengths, bond angles, torsion angles and displacement

parameters are listed in Tables 7-10 below.

### SOFTWARE AND REFERENCES

SHELXTL, Version 5.1, Bruker AXS, 1997
PLATON, A. L. Spek, J. Appl. Cryst. 2003, 36, 7-13.
MERCURY, C. F. Macrae, P. R. Edington, P. McCabe, E. Pidcock, G. P. Shields, R. Taylor, M. Towler and J. van de Streek, J. Appl. Cryst. 2006, 39, 453-457.

For structures with absolute configuration:
H. D. Flack, Acta Cryst. 1983, A39, 867-881.
R. W. W. Hooft, L. H. Straver, and A. L. Spek. J. Appl. Cryst. 2008, 41, 96-103.

TABLE 6
Crystal data and structure refinement for benzyl (2S,4S)-
4-hydroxy-2-methyl-4-(trichloromethyl)piperidine-1-carboxylate(C9)

| Empirical formula | C₁₅ H₁₈ Cl₃ N O₃ | |
|---|---|---|
| Formula Empirical weight | 366.65 | |
| Temperature | 298(2) K | |
| Wavelength | 1.54178 Å | |
| Crystal system | Orthorhombic | |
| Space group | P2(1)2(1)2(1) | |
| Unit cell dimensions | a = 8.14750(10) Å | α = 90° |
| | b = 11.0267(2) Å | β = 90° |
| | c = 19.1568(4) Å | γ = 90° |

| | |
|---|---|
| Volume | 1721.05(5) Å³ |
| Z | 4 |
| Density (calculated) | 1.415 Mg/m³ |
| Absorption coefficient | 4.919 mm⁻¹ |
| F(000) | 760 |
| Crystal size | 0.45 × 0.15 × 0.10 mm³ |
| Theta range for data collection | 4.62 to 67.34°. |
| Index ranges | −9 <= h <= 9, −12 <= k <= |
| | 10, −0 <= l <= 22 |
| Reflections collected | 7706 |
| Independent reflections | 2952 [R(int) = 0.0360] |
| Completeness to theta = 67.34° | 97.9% |
| Absorption correction | Empirical |
| Max. and min. transmission | 0.6390 and 0.2156 |
| Refinement method | Full-matrix least-squares on F² |
| Data/restraints/parameters | 2952/0/201 |
| Goodness-of-fit on F² | 1.025 |
| Final R indices [I > 2sigma(I)] | R1 = 0.0419, wR2 = 0.1061 |
| R indices (all data) | R1 = 0.0488, wR2 = 0.1119 |
| Absolute structure parameter | 0.023(19) |
| Largest diff. peak and hole | 0.196 and −0.298 e.Å⁻³ |

TABLE 7
Atomic coordinates (×10⁴) and equivalent isotropic displacement
parameters (Å² × 10³) for benzyl (2S,4S)-4-hydroxy-2-
methyl-4-(trichloromethyl)piperidine-1-carboxylate (C9). U(eq) is defined
as one third of the trace of the orthogonalized U^{ij} tensor.

| — | x | y | z | U(eq) |
|---|---|---|---|---|
| C(1) | 84(4) | 5621(3) | −305(2) | 57(1) |
| C(2) | −1585(5) | 5436(4) | −379(3) | 71(1) |
| C(3) | −2614(5) | 5765(4) | 159(3) | 74(1) |
| C(4) | −2038(5) | 6265(4) | 751(2) | 78(1) |
| C(5) | −336(5) | 6449(4) | 828(2) | 65(1) |
| C(6) | 732(4) | 6113(3) | 304(2) | 46(1) |
| C(7) | 2544(4) | 6254(3) | 386(2) | 55(1) |
| C(8) | 3814(3) | 5110(3) | 1304(2) | 40(1) |
| C(9) | 5285(4) | 3911(3) | 2159(2) | 44(1) |
| C(10) | 4115(4) | 3211(4) | 2622(2) | 59(1) |
| C(11) | 7038(3) | 3394(3) | 2105(2) | 43(1) |
| C(12) | 7213(3) | 2312(3) | 1614(2) | 40(1) |
| C(13) | 6464(4) | 2615(3) | 904(2) | 43(1) |
| C(14) | 4701(4) | 3029(3) | 988(2) | 46(1) |
| C(15) | 9058(4) | 1969(3) | 1525(2) | 51(1) |
| Cl(1) | 9949(1) | 1553(1) | 2337(1) | 71(1) |
| Cl(2) | 10241(1) | 3167(1) | 1163(1) | 82(1) |
| Cl(3) | 9279(1) | 688(1) | 962(1) | 79(1) |
| N(1) | 4613(3) | 4082(2) | 1455(1) | 42(1) |
| O(1) | 3271(3) | 5128(2) | 635(1) | 50(1) |
| O(2) | 3633(3) | 5963(2) | 1701(1) | 56(1) |
| O(3) | 6390(3) | 1268(2) | 1856(1) | 45(1) |

TABLE 8
Bond lengths [Å] and angles [°] for benzyl (2S,4S)-4-hydroxy-
2-methyl-4-(trichloromethyl)piperidine-1-carboxylate (C9).

| | |
|---|---|
| C(1)—C(2) | 1.382(5) |
| C(1)—C(6) | 1.391(5) |
| C(2)—C(3) | 1.377(6) |
| C(3)—C(4) | 1.346(6) |
| C(4)—C(5) | 1.409(6) |
| C(5)—C(6) | 1.378(5) |
| C(6)—C(7) | 1.493(4) |
| C(7)—O(1) | 1.457(4) |
| C(8)—O(2) | 1.219(4) |
| C(8)—N(1) | 1.338(4) |
| C(8)—O(1) | 1.355(3) |
| C(9)—N(1) | 1.468(4) |
| C(9)—C(10) | 1.513(4) |
| C(9)—C(11) | 1.541(4) |
| C(11)—C(12) | 1.526(4) |
| C(12)—O(3) | 1.411(3) |
| C(12)—C(13) | 1.527(4) |
| C(12)—C(15) | 1.559(4) |
| C(13)—C(14) | 1.516(4) |
| C(14)—N(1) | 1.468(4) |
| C(15)—Cl(2) | 1.776(4) |
| C(15)—Cl(1) | 1.777(3) |
| C(15)—Cl(3) | 1.786(4) |
| C(2)—C(1)—C(6) | 121.1(4) |
| C(3)—C(2)—C(1) | 118.9(4) |
| C(4)—C(3)—C(2) | 121.8(4) |
| C(3)—C(4)—C(5) | 119.3(4) |
| C(6)—C(5)—C(4) | 120.5(4) |
| C(5)—C(6)—C(1) | 118.4(3) |
| C(5)—C(6)—C(7) | 121.3(3) |
| C(1)—C(6)—C(7) | 120.2(3) |
| O(1)—C(7)—C(6) | 110.3(3) |
| O(2)—C(8)—N(1) | 125.3(3) |
| O(2)—C(8)—O(1) | 122.6(3) |
| N(1)—C(8)—O(1) | 112.0(2) |
| N(1)—C(9)—C(10) | 111.7(3) |
| N(1)—C(9)—C(11) | 109.3(2) |
| C(10)—C(9)—C(11) | 115.8(3) |
| C(12)—C(11)—C(9) | 114.7(2) |
| O(3)—C(12)—C(11) | 112.9(2) |
| O(3)—C(12)—C(13) | 106.4(2) |
| C(11)—C(12)—C(13) | 109.9(2) |
| O(3)—C(12)—C(15) | 107.2(2) |
| C(11)—C(12)—C(15) | 110.3(2) |
| C(13)—C(12)—C(15) | 110.0(2) |
| C(14)—C(13)—C(12) | 110.5(2) |
| N(1)—C(14)—C(13) | 110.4(2) |
| C(12)—C(15)—Cl(2) | 112.7(2) |
| C(12)—C(15)—Cl(1) | 111.2(2) |
| Cl(2)—C(15)—Cl(1) | 108.17(18) |
| C(12)—C(15)—Cl(3) | 110.8(2) |
| Cl(2)—C(15)—Cl(3) | 107.35(17) |
| Cl(1)—C(15)—Cl(3) | 106.44(19) |
| C(8)—N(1)—C(9) | 119.3(2) |
| C(8)—N(1)—C(14) | 124.2(2) |
| C(9)—N(1)—C(14) | 116.1(2) |
| C(8)—O(1)—C(7) | 117.1(2) |

Symmetry transformations used to generate equivalent atoms.

TABLE 9
Anisotropic displacement parameters (Å² × 10³) for benzyl (2S,4S)-
4-hydroxy-2-methyl-4-(trichloromethyl)piperidine-1-carboxylate
(C9). The anisotropic displacement factor exponent takes the
form: −2π²[h²a*²U¹¹ + . . . + 2 h k a* b* U¹²]

| | U¹¹ | U²² | U³³ | U²³ | U¹³ | U¹² |
|---|---|---|---|---|---|---|
| C(1) | 53(2) | 56(2) | 62(2) | 1(2) | −4(2) | 8(2) |
| C(2) | 59(2) | 64(2) | 90(3) | −2(2) | −18(2) | −5(2) |
| C(3) | 46(2) | 68(2) | 109(4) | 24(2) | −2(2) | −3(2) |
| C(4) | 66(2) | 85(3) | 83(3) | 22(2) | 26(2) | 15(2) |
| C(5) | 73(2) | 72(2) | 50(2) | 10(2) | 1(2) | 15(2) |
| C(6) | 44(2) | 41(2) | 55(2) | 14(1) | −2(1) | 5(1) |
| C(7) | 49(2) | 52(2) | 64(2) | 18(2) | −12(2) | 3(1) |
| C(8) | 31(1) | 46(2) | 43(2) | 3(1) | −1(1) | 0(1) |
| C(9) | 47(2) | 45(2) | 40(1) | −3(1) | −6(1) | 4(1) |
| C(10) | 48(2) | 74(2) | 54(2) | 8(2) | 11(2) | 14(2) |
| C(11) | 39(1) | 48(2) | 42(2) | −1(1) | −6(1) | 0(1) |
| C(12) | 35(1) | 44(2) | 42(2) | 4(1) | 0(1) | −1(1) |
| C(13) | 51(2) | 43(2) | 37(2) | −1(1) | −1(1) | 4(1) |
| C(14) | 51(2) | 42(2) | 44(2) | −2(1) | −12(1) | 1(1) |
| C(15) | 39(2) | 67(2) | 47(2) | 3(2) | 3(1) | 4(1) |
| Cl(1) | 49(1) | 101(1) | 61(1) | 7(1) | −12(1) | 18(1) |
| Cl(2) | 45(1) | 103(1) | 99(1) | 25(1) | 14(1) | −15(1) |
| Cl(3) | 65(1) | 93(1) | 80(1) | −25(1) | 2(1) | 30(1) |
| N(1) | 42(1) | 44(1) | 41(1) | −3(1) | −6(1) | 5(1) |
| O(1) | 50(1) | 50(1) | 50(1) | 6(1) | −12(1) | 7(1) |
| O(2) | 67(1) | 47(1) | 54(1) | −6(1) | −5(1) | 14(1) |
| O(3) | 45(1) | 42(1) | 50(1) | 6(1) | −3(1) | −5(1) |

TABLE 10
Hydrogen coordinates (×10⁴) and isotropic displacement
parameters (Å² × 10³) for benzyl (2S,4S)-4-hydroxy-
2-methyl-4-(trichloromethyl)piperidine-1-carboxylate (C9).

| | x | y | z | U(eq) |
|---|---|---|---|---|
| H(1) | 786 | 5412 | −668 | 68 |
| H(2) | −2006 | 5096 | −786 | 85 |
| H(3A) | −3737 | 5638 | 112 | 89 |
| H(4) | −2756 | 6487 | 1106 | 93 |
| H(5) | 70 | 6799 | 1234 | 78 |
| H(7A) | 3027 | 6472 | −60 | 66 |
| H(7B) | 2773 | 6901 | 714 | 66 |
| H(9) | 5389 | 4721 | 2365 | 53 |
| H(10A) | 3096 | 3644 | 2658 | 88 |
| H(10B) | 4588 | 3120 | 3078 | 88 |
| H(10C) | 3918 | 2425 | 2424 | 88 |
| H(11A) | 7766 | 4033 | 1947 | 52 |
| H(11B) | 7396 | 3150 | 2567 | 52 |
| H(13A) | 7100 | 3251 | 682 | 52 |
| H(13B) | 6500 | 1904 | 606 | 52 |
| H(14A) | 4253 | 3243 | 535 | 55 |
| H(14B) | 4047 | 2372 | 1178 | 55 |
| H(3) | 6524 | 1203 | 2279 | 68 |

### Step 2. Synthesis of 1-benzyl 4-methyl (2S,4R)-4-azido-2-methylpiperidine-1,4-dicarboxylate (C10)

A suspension of benzyl (2S,4S)-4-hydroxy-2-methyl-4-(trichloromethyl)piperidine-1-carboxylate (C9) (18.00 g, 49.09 mmol), 18-crown-6 ether (2.00 g, 7.57 mmol) and sodium azide (98%, 9.00 g, 136 mmol) in methanol (130 mL) was stirred at room temperature for 1 hour. 1,8-Diazabicyclo[5.4.0]undec-7-ene (98%, 24.0 mL, 157 mmol) was then added over ten minutes. The reaction mixture was stirred at room temperature for 18 hours. Most of the methanol was removed in vacuo, and the residue was diluted with water (200 mL) and extracted with ethyl acetate (2×250 mL). The combined organic extracts were washed with water (150 mL), washed with saturated aqueous sodium chloride solution (150 mL) and dried over magnesium sulfate. After filtration and removal of solvent under reduced pressure, C10 was obtained as a light yellow oil. Yield: 15.8 g, 47.5 mmol, 97%. APCI m/z 333.3 (M+1). ¹H NMR (400 MHz, CDCl₃) δ 1.09 (d, J=7.1 Hz, 3H), 1.60 (ddd, J=13.5, 12.5, 5.3 Hz, 1H), 1.94 (dd, J=13.6, 6.1 Hz, 1H), 2.23-2.32 (m, 2H), 3.16 (ddd, J=14.3, 12.3, 3.2 Hz, 1H), 3.84 (s, 3H), 4.07 (br ddd, J=14, 5, 3 Hz, 1H), 4.45-4.53 (m, 1H), 5.14 (s, 2H), 7.30-7.40 (m, 5H).

### Step 3. Synthesis of 1-benzyl 4-methyl (2S,4R)-4-amino-2-methylpiperidine-1,4-dicarboxylate, hydrochloride salt (C11)

Zinc dust (99%, 4.76 g, 72 mmol) was added to a solution of compound 1-benzyl 4-methyl (2S,4R)-4-azido-2-methylpiperidine-1,4-dicarboxylate (C10) (4.8 g, 14.4 mmol) in acetic acid (35 mL) and tetrahydrofuran (35 mL), and the reaction mixture was heated at 50° C. for 4 hours. After cooling to room temperature, the mixture was filtered through Celite, and the filtrate was concentrated in vacuo to remove most of the solvents. The residue was diluted with ethyl acetate, washed several times with saturated aqueous sodium bicarbonate solution, then washed with saturated aqueous sodium chloride solution and dried over magnesium sulfate. The mixture was filtered and concentrated under reduced pressure to provide the free base of the product as a light yellow oil. Yield: 4.4 g, 14.4 mmol, quantitative. LCMS m/z 307.5 (M+1). ¹H NMR (400 MHz, CDCl₃) δ 1.05 (d, J=7.1 Hz, 3H), 1.44 (ddd, J=13.2, 12.8, 5.2 Hz, 1H), 1.73 (dd, J=13.6, 6.0 Hz, 1H), 2.15-2.26 (m, 4H), 3.16 (ddd, J=14.1, 12.7, 3.1 Hz, 1H), 3.75 (s, 3H), 4.05 (br ddd, J=14, 5, 3 Hz, 1H), 4.42-4.50 (m, 1H), 5.14 (AB quartet, J_{AB}=12.5 Hz, Δν_{AB}=5.5 Hz, 2H), 7.29-7.39 (m, 5H). This material can be converted to its hydrochloride salt by dissolution in a 5:1 mixture of diethyl ether and methanol, and treatment of the solution with an excess of a solution of hydrogen chloride in diethyl ether. The title compound is isolated by filtration as a white solid. APCI m/z 307.3 (M+1). ¹H NMR (400 MHz, DMSO-d₆) δ 8.99 (br s, 3H), 7.29-7.40 (m, 5H), 5.09 (AB quartet, J_{AB}=12.6 Hz, Δν_{AB}=14.0 Hz, 2H), 4.33-4.42 (m, 1H), 3.99 (br ddd, J=14, 5, 3 Hz, 1H), 3.78 (s, 3H), 3.17-3.25 (m, 1H), 2.27 (br d, J=13.5 Hz, 1H), 2.13-2.18 (m, 1H), 2.07 (dd, half of ABX pattern, J=14.0, 6.0 Hz, 1H), 1.82 (ddd, J=13.0, 13.0, 5.2 Hz, 1H), 1.00 (d, J=7.0 Hz, 3H).

### Step 4. Synthesis of benzyl (2S,4R)-4-amino-4-(hydroxymethyl)-2-methylpiperidine-1-carboxylate (C12)

Sodium borohydride (24.1 g, 0.64 mol) was suspended in ethanol (500 mL) and the flask was cooled with a water bath. 1-Benzyl 4-methyl (2S,4R)-4-amino-2-methylpiperidine-1,4-dicarboxylate, hydrochloride salt (C11) (25.0 g, 73.0 mmol) was added in portions, while maintaining the temperature below 30° C. The suspension was stirred at room temperature for 18 hours, at which time aqueous 5 N hydrochloric acid was added to bring the pH to 7, and the slurry was concentrated in vacuo. Water (50 mL) was added to the residue, and the resulting mixture was extracted with ethyl acetate (4×200 mL). The combined extracts were washed with water (2×250 mL), then with saturated aqueous sodium chloride solution and dried over sodium sulfate. Filtration and removal of solvent in vacuo provided the product as a clear oil. Yield: 18.65 g, 67.00 mmol, 92%. This product was used in the next step without purification. LCMS m/z 279.2 (M+1). ¹H NMR (300 MHz, CDCl₃) δ 7.31-7.39 (m, 5H), 5.15 (AB quartet, J_{AB}=12.5 Hz, Δν_{AB}=7.4 Hz, 2H), 4.28-4.37 (m, 1H), 4.00 (br ddd, J=14, 6, 3 Hz, 1H), 3.48 (AB quartet, J_{AB}=10.7 Hz, Δν_{AB}=36.5 Hz, 2H), 3.03 (ddd, J=14.2, 12.0, 4.0 Hz, 1H), 1.57-1.83 (m, 3H), 1.40-1.52 (m, 1H), 1.21 (d, J=6.8 Hz, 3H).

### Step 5. Synthesis of benzyl (2S,4R)-4-amino-4-({[tert-butyl(dimethyl)sily]oxy}methyl)-2-methylpiperidine-1-carboxylate (C13)

Benzyl (2S,4R)-4-amino-4-(hydroxymethyl)-2-methylpiperidine-1-carboxylate (C12) (18.65 g, 67.00 mmol) was dissolved in dichloromethane (350 mL). Triethylamine (20.8 mL, 149 mmol), 4-(dimethylamino)pyridine (81 mg, 0.66 mmol) and tert-butyldimethylsilyl chloride (11.15 g, 74.0 mmol) were added and the solution was stirred at room temperature for 18 hours. Water (350 mL) was added and the mixture was stirred for 20 min. The layers were separated and the organic fraction was washed with water (2×200 mL) and saturated aqueous sodium chloride solution, then dried over sodium sulfate, filtered and concentrated in vacuo to yield the product as a clear oil. Yield: 24.3 g, 61.9 mmol, 92%. The crude product was used in the next step without purification. LCMS m/z 393.2 (M+1). ¹H NMR (300 MHz, CDCl₃) δ 7.31-7.38 (m, 5H), 5.14 (AB quartet, J_{AB}=12.4 Hz, Δν_{AB}=8.4 Hz, 2H), 4.22-4.37 (m, 1H), 3.94 (br ddd, J=14, 6, 3 Hz, 1H), 3.46 (AB quartet, J_{AB}=9.5 Hz, Δν_{AB}=11.8 Hz, 2H), 3.02 (ddd, J=14.0, 11.7, 4.1 Hz, 1H), 1.58-1.73 (m, 3H), 1.39-1.50 (m, 1H), 1.20 (d, J=6.8 Hz, 3H), 0.91 (s, 9H), 0.07 (s, 6H).

### Step 6. Synthesis of benzyl (2S,4R)-4-({[tert-butyl(dimethyl)silyl]oxy}methyl)-4-{[(2-methoxy-2-oxoethyl)sulfonyl]amino}-2-methylpiperidine-1-carboxylate (C14)

Benzyl (2S,4R)-4-amino-4-({[tert-butyl(dimethyl)silyl]oxy}methyl)-2-methylpiperidine-1-carboxylate (C13) (24.3 g, 61.9 mmol) was dissolved in tetrahydrofuran (300 mL). 2,4,6-Collidine (99%, 11.0 mL, 82.4 mmol), 4-(dimethylamino)pyridine (75 mg, 0.61 mmol) and methyl (chlorosulfonyl)acetate (11.9 g, 68.9 mmol) were added. The mixture was stirred at room temperature for 66 hours, at which time the volatiles were removed in vacuo and the residue was taken up in ethyl acetate (500 mL). The solution was washed with aqueous 1 N potassium hydrogensulfate solution (2×250 mL) and saturated aqueous sodium chloride solution, then dried over sodium sulfate, filtered and concentrated in vacuo. Purification using silica gel chromatography (Gradient: 50% to 100% ethyl acetate in heptanes) gave the product as a yellow oil. Yield: 5.1 g, 9.6 mmol, 16%. LCMS m/z 529.1 (M+1). ¹H NMR (300 MHz, CDCl₃) δ 7.31-7.38 (m, 5H), 5.13 (AB quartet, J_{AB}=12.3 Hz, Δν_{AB}=9.8 Hz, 2H), 4.96 (br s, 1H), 4.25-4.37 (m, 1H), 4.07 (s, 2H), 3.94-4.02 (m, 1H), 3.78 (s, 3H), 3.76 (AB quartet, J_{AB}=10.4 Hz, Δν_{AB}=7.0 Hz, 2H), 3.05 (ddd, J=14.2, 11.2, 4.3 Hz, 1H), 2.15 (dd, J=14.2, 6.5 Hz, 1H), 1.97 (ddd, J=14.0, 11.2, 5.6 Hz, 1H), 1.82 (ddd, J=14.0, 4, 4 Hz, 1H), 1.70 (dd, J=14, 6 Hz, 1H), 1.22 (d, J=6.8 Hz, 3H), 0.91 (s, 9H), 0.10 (s, 6H).

### Step 7. Synthesis of benzyl (2S,4R)-4-(hydroxymethyl)-4-{([(2-methoxy-2-oxoethyl)sulfonyl]-amino}-2-methylpiperidine-1-carboxylate (C15)

Benzyl (2S,4R)-4-({[tert-butyl(dimethyl)silyl]oxy}methyl)-4-{[(2-methoxy-2-oxoethyl)sulfonyl]amino}-2-methylpiperidine-1-carboxylate (C14) (5.1 g, 9.6 mmol) was dissolved in tetrahydrofuran (100 mL). Tetrabutylammonium fluoride solution (1 M in tetrahydrofuran, 14.2 mL, 14.2 mmol) was added drop-wise to this solution over 10 minutes. After the addition was complete, the mixture was stirred at room temperature for 2 hours. Volatiles were removed in vacuo and the residue was taken up in ethyl acetate (350 mL). The solution was washed with water (2×70 mL) and saturated aqueous sodium chloride solution, then dried over sodium sulfate, filtered and concentrated in vacuo. Silica gel chromatography (Eluant: ethyl acetate) afforded the product as a yellow oil. Yield: 2.4 g, 5.8 mmol, 60%. The ¹H NMR indicated that this material was a roughly 3:2 mixture of rotamers. LCMS m/z 415.1 (M+1). ¹H NMR (300 MHz, CDCl₃), selected peaks, δ 7.29-7.41 (m, 5H), 5.88 (br s, 1H), 5.09-5.19 (m, 2H), 4.44-4.58 (m, 2H), 3.01-3.16 (m, 1H), 2.22 (dd, J=14.3, 6.3 Hz) and 2.12 (dd, J=14.1, 6.6 Hz, total 1H), 1.23 (d, J=6.8 Hz) and 1.22 (d, J=6.8 Hz, total 3H).

### Step 8. Synthesis of benzyl (2S,4R)-4-formyl-4-{([(2-methoxy-2-oxoethyl)sulfonyl]amino}-2-methylpiperidine-1-carboxylate (C16)

Benzyl (2S,4R)-4-(hydroxymethyl)-4-{[(2-methoxy-2-oxoethyl)sulfonyl]amino}-2-methylpiperidine-1-carboxylate (C15) (0.76 g, 1.83 mmol) was dissolved in dichloromethane (25 mL). A solution of Dess-Martin periodinane (15% by weight in dichloromethane, 5.69 g, 2.01 mmol) was added and the mixture was stirred for 18 hours. Saturated aqueous sodium bicarbonate solution (25 mL) was added and the layers were separated. The organic layer was washed with saturated aqueous sodium bicarbonate solution (2×10 mL), dried over sodium sulfate, filtered and concentrated in vacuo. Silica gel chromatography (Eluant: 2:1 ethyl acetate in heptane) provided the product as a yellow oil. Yield: 0.41 g, 0.99 mmol, 54%. The product seemed by NMR to be a mixture of rotamers. ¹H NMR (300 MHz, CDCl₃), selected peaks, δ 9.65 (d, J=0.9 Hz, 1H), 7.34-7.39 (m, 5H), 5.10-5.19 (m, 2H), 3.18 (ddd, J=14.5, 12.0, 3.8 Hz, 1H), 1.16 (d, J=6.9 Hz) and 1.26 (d, J=6.8 Hz, total 3H).

### Step 9. Synthesis of 8-benzyl 3-methyl (5R,7S)-7-methyl-2-thia-1,8-diazaspiro[4.5]dec-3-ene-3,8-dicarboxylate 2,2-dioxide (C17)

Benzyl (2S,4R)-4-formyl-4-{[(2-methoxy-2-oxoethyl)sulfonyl]amino}-2-methylpiperidine-1-carboxylate (C16) (0.41 g, 0.99 mmol) was dissolved in ethanol (2 mL). Piperidine (2 drops) was added and the mixture was stirred at 65° C. for 45 min. The volatiles were removed in vacuo; chromatography on silica gel (Eluant: 1:1 ethyl acetate in heptane) gave the product as an off-white solid. Yield: 0.27 g, 0.68 mmol, 69%. LCMS m/z 393.1 (M−1). ¹H NMR (300 MHz, CDCl₃) δ 7.70 (s, 1H), 7.34-7.42 (m, 5H), 5.16 (AB quartet, J_{AB}=12.4 Hz, Δν_{AB}=4.6 Hz, 2H), 4.62 (br s, 1H), 4.41-4.52 (m, 1H), 4.07-4.15 (m, 1H), 3.95 (s, 3H), 3.15-3.26 (m, 1H), 2.03-2.11 (m, 1H), 1.90-2.02 (m, 2H), 1.85 (brdd, J=13.9, 5.2 Hz, 1H), 1.29 (d, J=7.0 Hz, 3H)

### Step 10. Synthesis of benzyl (5R,7S)-7-methyl-2-thia-1,8-diazaspiro[4.5]dec-3-ene-8-carboxylate 2,2-dioxide (C18)

8-Benzyl 3-methyl (5R,7S)-7-methyl-2-thia-1,8-diazaspiro[4.5]dec-3-ene-3,8-dicarboxylate 2,2-dioxide (C17) (0.22 g, 0.56 mmol) was dissolved in dimethyl sulfoxide (1.52 mL) in a pressure tube. Water (0.11 mL) and sodium chloride (39 mg, 0.67 mmol) were added. The tube was sealed and the mixture was stirred and heated at 165° C. for 6 hours. After cooling to room temperature, the reaction was treated with saturated aqueous sodium chloride solution (6 mL) and the mixture was extracted with ethyl acetate (3×7 mL). The combined extracts were dried over sodium sulfate, filtered and concentrated in vacuo. Silica gel chromatography (Eluant: 1:1 ethyl acetate:heptane) afforded the product as a yellow oil. Yield: 67 mg, 0.20 mmol (36%). LCMS m/z 335.1 (M−1). ¹H NMR (300 MHz, CDCl₃) δ 7.33-7.40 (m, 5H), 6.92 (d, J=6.5 Hz, 1H), 6.71 (d, J=6.5 Hz, 1H), 5.16 (s, 2H), 4.39-4.49 (m, 1H), 4.04-4.11 (m, 1H), 3.14-3.24 (m, 1H), 2.04 (dd, J=13.8, 6.3 Hz, 1H), 1.90-1.97 (m, 2H), 1.77-1.84 (m, 1H), 1.27 (d, J=6.9 Hz, 3H).

### Preparation 2: tert-Butyl (5R,7S)-7-methyl-2-thia-1,8-diazaspiro[4.5]dec-3-ene-8-carboxylate 2,2-dioxide (P2)

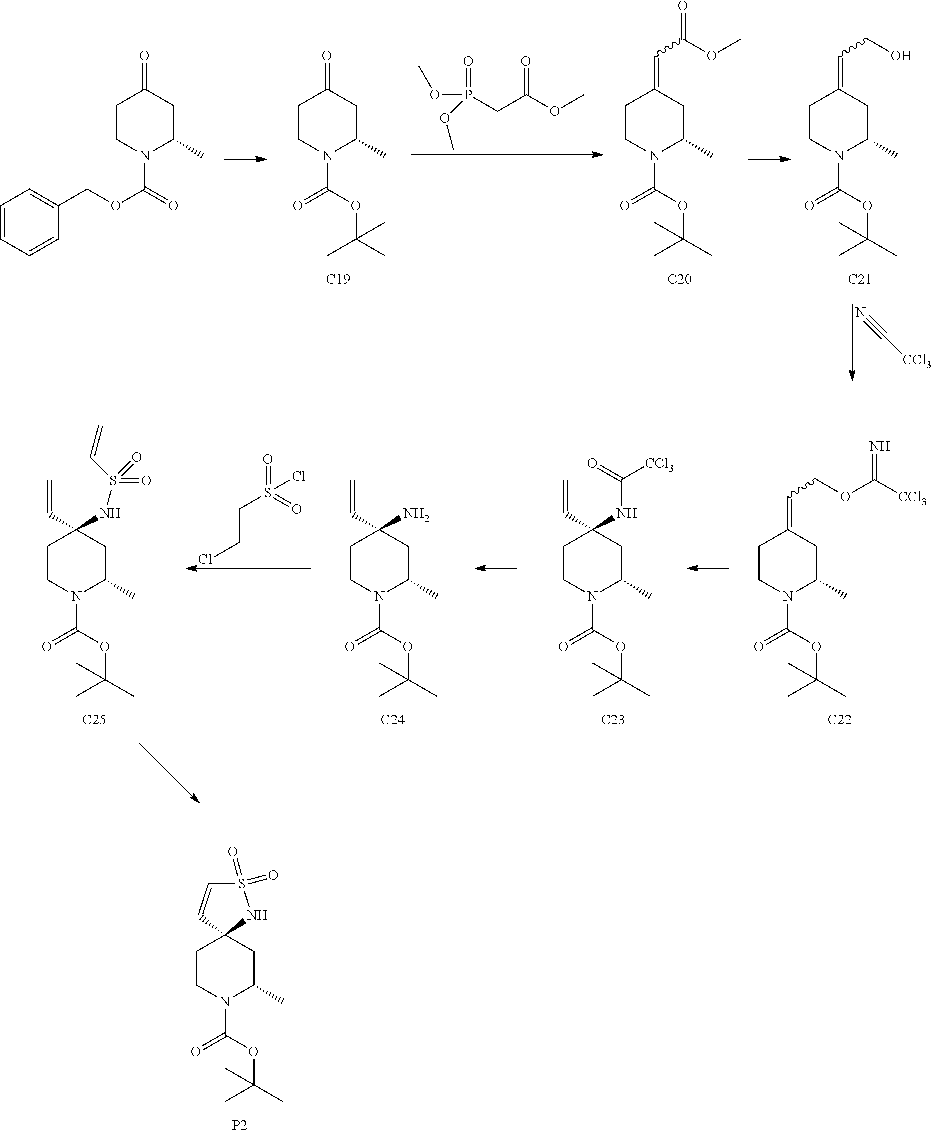

### Step 1. Synthesis of tert-butyl (2S)-2-methyl-4-oxopiperidine-1-carboxylate (C19)

A mixture of benzyl (2S)-2-methyl-4-oxopiperidine-1-carboxylate (6.00 g, 24.3 mmol), palladium on carbon (1.03 g), ethanol (50 mL) and tetrahydrofuran (50 mL) was treated with di-tert-butyl dicarbonate (5.82 g, 26.7 mmol) and subjected to Parr hydrogenation at 15 psi for 18 hours. The reaction was filtered through Celite and the filter cake was washed with ethanol (3×150 mL). The combined filtrates were concentrated in vacuo, yielding an oily residue that crystallized when placed under high vacuum. The product was obtained as a solid. Yield: 5.52 g, 25.9 mmol, quantitative. APCI m/z 114.0 [(M-tert-BOC)+1]. ¹H NMR (400 MHz, CDCl₃) δ 4.67-4.75 (m, 1H), 4.20-4.27 (m, 1H), 3.32 (ddd, J=13.9, 11.2, 4.0 Hz, 1H), 2.68 (dd, J=14.5, 6.7 Hz, 1H), 2.48 (br ddd, J=15.3, 11.3, 6.9 Hz, 1H), 2.31-2.38 (m, 1H), 2.25 (ddd, J=14.5, 2.7, 1.8 Hz, 1H), 1.49 (s, 9H), 1.18 (d, J=6.8 Hz, 3H).

### Step 2. Synthesis of tert-butyl (2S,4E)- and tert-butyl (2S,4Z)-4-(2-methoxy-2-oxoethylidene)-2-methylpiperidine-1-carboxylate (C20)

Sodium hydride (60% in mineral oil, 1.35 g, 33.6 mmol) was washed with hexanes (2×5 mL), suspended in N,N-dimethylformamide (40 mL) and cooled to 0° C. Methyl (dimethoxyphosphoryl)acetate (4.66 mL, 32.3 mmol) was added to the reaction in a drop-wise manner, and the mixture was held at 0° C. with vigorous stirring for 20 minutes. A solution of tert-butyl (2S)-2-methyl-4-oxopiperidine-1-carboxylate (C19) (5.52 g from the previous experiment, ≦24.3 mmol) in N,N-dimethylformamide (10 mL) was added drop-wise, and the resulting solution was allowed to warm to room temperature over 16 hours. The reaction was then diluted with diethyl ether (400 mL) and washed with water (300 mL). The aqueous layer was extracted with diethyl ether (200 mL) and the combined organic layers were washed with water (4×200 mL) and saturated aqueous sodium chloride solution (200 mL), then dried over magnesium sulfate, filtered and concentrated under reduced pressure. The product was obtained as a colorless oil, composed of a roughly 1:1 mixture of olefin isomers. Yield: 6.63 g, 24.6 mmol, quantitative. ¹H NMR (400 MHz, CDCl₃) δ 5.83 and 5.72 (2 br s, 1H), 4.44-4.61 (m, 1H), 3.98-4.14 (m, 1H), 3.71 and 3.70 (2 s, 3H), 3.58-3.70 (m, 1H), 2.93-3.03 (m, 1H), 2.06-2.11, 2.18-2.33 and 2.53-2.59 (multiplets, total 3H), 1.47 (2 s, 9H), 1.08 (d, J=6.7 Hz) and 1.07 (d, J=6.9 Hz, total 3H).

### Step 3. Synthesis of tert-butyl (2S,4E)- and tert-butyl (2S,4Z)-4-(2-hydroxyethylidene)-2-methylpiperidine-1-carboxylate (C21)

A solution of tert-butyl (2S,4E)- and tert-butyl (2S,4Z)-4-(2-methoxy-2-oxoethylidene)-2-methylpiperidine-1-carboxylate (C20) (2.91 g, 10.8 mmol) in toluene (75 mL) was cooled to −78° C. and treated drop-wise with diisobutylaluminum hydride (1.5 M in toluene, 18.0 mL, 27.0 mmol). The reaction was maintained at −78° C. for 18 hours, then quenched with methanol (0.5 mL), warmed to room temperature and stirred for 2 hours. After filtration through Celite and washing of the filter cake with ethyl acetate (3×100 mL), the combined filtrates were concentrated in vacuo, and the residue was purified by silica gel chromatography (Gradient: 30% to 50% ethyl acetate in heptane). The product was obtained as a colorless oil, judged by ¹H NMR analysis to consist of a roughly 1:1 mixture of olefin isomers. Yield: 2.19 g, 9.07 mmol, 84%. APCI m/z 142.0 [(M-tert-BOC)+1]. ¹H NMR (400 MHz, CDCl₃) δ 5.60-5.65 and 5.46-5.51 (m, 1H), 4.39-4.57 (m, 1H), 4.12-4.21 (m, 2H), 3.98-4.06 (m, 1H), 2.82-2.93 (m, 1H), 2.36-2.54 and 2.10-2.21 (m, 3H), 1.95-2.03 (m, 1H), 1.47 (s, 9H), 1.28-1.35 (m, 1H), 1.03-1.07 (m, 3H).

### Step 4. Synthesis of tert-butyl (2S,4E)- and tert-butyl (2S,4Z)-2-methyl-4-{2-[(2,2,2-trichloroethanimidoyl)oxy]ethylidene}piperidine-1-carboxylate (C22)

Trichloroacetonitrile (1.37 mL, 13.7 mmol) was added to a 0° C. solution of tert-butyl (2S,4E)- and tert-butyl (2S,4Z)-4-(2-hydroxyethylidene)-2-methylpiperidine-1-carboxylate (C21) (2.19 g, 9.07 mmol) and 1,8-diazabicyclo[5.4.0]undec-7-ene (95%, 0.287 mL, 1.82 mmol) in dichloromethane (65 mL). The resulting solution was allowed to warm slowly to room temperature. After 2 hours, volatiles were removed in vacuo, and the residue was purified by silica gel chromatography (Gradient: 0% to 30% ethyl acetate in heptane). The product was obtained as a colorless oil, judged by ¹H NMR analysis to consist of a roughly 1:1 mixture of olefin isomers. Yield: 3.32 g, 8.61 mmol, 95%. ¹H NMR (400 MHz, CDCl₃) δ 8.29 (br s, 1H), 5.69-5.74 and 5.53-5.58 (m, 1H), 4.77-4.92 (m, 2H), 4.42-4.60 (m, 1H), 4.00-4.09 (m, 1H), 2.85-2.96 (m, 1H), 2.58-2.64, 2.43-2.51, 2.16-2.28 and 2.00-2.09 (4 multiplets, total 4H), 1.47 (s, 9H), 1.06 (2 d, J=6.8 Hz, 3H).

### Step 5. Synthesis of tert-butyl (2S,4R)-2-methyl-4-[(trichloroacetyl)amino]-4-vinylpiperidine-1-carboxylate (C23)

Potassium carbonate (10 g, 72 mmol) was added to a solution of tert-butyl (2S,4E)- and tert-butyl (2S,4Z)-2-methyl-4-{2-[(2,2,2-trichloroethanimidoyl)oxy]ethylidene}piperidine-1-carboxylate (C22) (3.22 g, 8.35 mmol) in xylenes (350 mL), and the mixture was heated to 140° C. for 72 hours. The reaction was cooled and concentrated in vacuo, treatment of the residue with diethyl ether (20 mL) caused a solid to precipitate. Isolation of this solid by filtration and washing with diethyl ether (2×10 mL) provided the product as a white solid (1.16 g). Removal of solvent from the filtrate under reduced pressure provided an oil, which was subjected to chromatography on silica gel (Gradient: 0% to 60% ethyl acetate in heptane) to provide additional product as a white solid. The cis orientation of the methyl and vinyl groups was established by observation of a nuclear Overhauser effect between these substituents in the proton NMR. Total yield: 1.48 g, 3.84 mmol, 46%. ¹H NMR (400 MHz, CDCl₃) δ 6.53 (br s, 1H), 6.07 (dd, J=17.6, 10.7 Hz, 1H), 5.30 (d, J=17.5 Hz, 1H), 5.28 (d, J=10.7 Hz, 1H), 4.18-4.26 (m, 1H), 4.01 (br ddd, J=14.2, 6.2, 2.6 Hz, 1H), 3.05 (ddd, J=14.2, 11.7, 4.5 Hz, 1H), 2.40 (dd, J=13.7, 6.6 Hz, 1H), 2.15-2.21 (m, 1H), 1.85-1.94 (m, 2H), 1.45 (s, 9H), 1.18 (d, J=6.7 Hz, 3H).

### Step 6

Synthesis of tert-butyl (2S,4R)-4-amino-2-methyl-4-vinylpiperidine-1-carboxylate (C24). Diisobutylaluminum hydride (1.5 M in toluene, 0.124 mL, 0.186 mmol) was added to a −78° C. solution of tert-butyl (2S,4R)-2-methyl-4-[(trichloroacetyl)amino]-4-vinylpiperidine-1-carboxylate (C23) (47.7 mg, 0.124 mmol) in dichloromethane (2.5 mL), and the reaction was maintained at this temperature for 1 hour. Ethyl acetate (4 mL) was added to the cold reaction, followed by a saturated aqueous solution of potassium sodium tartrate (10 mL). Additional ethyl acetate (15 mL) was added, and the reaction was allowed to warm to room temperature and stir for 1.5 hours. The aqueous layer was extracted with ethyl acetate (2×25 mL) and the combined organic layers were dried over sodium sulfate, filtered and concentrated in vacuo. Purification by silica gel chromatography (Gradient: 3.5% to 10% methanol in dichloromethane) provided the product as an oil. Yield: 22 mg, 0.092 mmol, 74%. LCMS m/z 241.2 (M+1). ¹H NMR (400 MHz, CDCl₃) δ 6.02 (dd, J=17.6, 10.9 Hz, 1H), 5.26 (d, J=17.7 Hz, 1H), 5.16 (d, J=10.8 Hz, 1H), 4.31-4.40 (m, 1H), 3.95 (br ddd, J=14, 4, 4 Hz, 1H), 2.99 (ddd, J=13.9, 12.5, 3.0 Hz, 1H), 2.20 (br s, 2H), 1.93-1.99 (m, 1H), 1.77 (dd, half of ABX pattern, J=13.6, 6.0 Hz, 1H), 1.72 (ddd, half of ABXY pattern, J=13.5, 3.5, 1.7 Hz, 1H), 1.56 (ddd, J=13, 13, 5 Hz, 1H), 1.46 (s, 9H), 1.15 (d, J=7.1 Hz, 3H).

### Step 7

Synthesis of tert-butyl (2S,4R)-2-methyl-4-vinyl-4-[(vinylsulfonyl)amino]piperidine-1-carboxylate (C25). A solution of tert-butyl (2S,4R)-4-amino-2-methyl-4-vinylpiperidine-1-carboxylate (C24) (59 mg, 0.24 mmol) in dichloromethane (2 mL) and pyridine (2 mL) was cooled to 0° C. and treated drop-wise with 2-chloroethanesulfonyl chloride (96%, 27.0 μL, 0.248 mmol). The mixture was stirred at 0° C. for 15 minutes, than allowed to warm to room temperature and stirred for 18 hours. The reaction was diluted with aqueous citric acid (1 M, 10 mL) and extracted with ethyl acetate (2×25 mL). The combined organic layers were washed with saturated aqueous sodium chloride solution (20 mL), dried over sodium sulfate, filtered and concentrated in vacuo. Chromatography on silica gel (Gradient: 0% to 100% ethyl acetate in heptane) afforded the product as a colorless oil. Yield: 37 mg, 0.11 mmol, 44%. LCMS m/z 329.1 (M−1). ¹H NMR (500 MHz, CDCl₃) δ 6.54 (dd, J=16.6, 9.8 Hz, 1H), 6.15 (d, J=16.6 Hz, 1H), 5.96 (dd, J=17.8, 10.7 Hz, 1H), 5.81 (d, J=9.8 Hz, 1H), 5.29-5.36 (m, 2H), 4.55 (s, 1H), 4.34 (td, J=6.8, 3.7 Hz, 1H), 3.88-3.96 (m, 1H), 2.96 (ddd, J=14.0, 12.3, 3.2 Hz, 1H), 2.18-2.25 (m, 1H), 2.07 (dd, J=13.5, 6.5 Hz, 1H), 1.87-1.93 (m, 1H), 1.83 (td, J=12.9, 5.1 Hz, 1H), 1.45 (s, 9H), 1.13 (d, J=7.1 Hz, 3H).

### Step 8

Synthesis of tert-butyl (5R,7S)-7-methyl-2-thia-1,8-diazaspiro[4.5]dec-3-ene-8-carboxylate 2,2-dioxide (P2). A mixture of tert-butyl (2S,4R)-2-methyl-4-vinyl-4-[(vinylsulfonyl)amino]piperidine-1-carboxylate (C25) (36 mg, 0.11 mmol), 1,3-bis-(2,4,6-trimethylphenyl)-2-imidazolidinylidene)dichloro(phenylmethylene)(tricyclohexylphosphine)ruthenium (4.2 mg, 0.0050 mmol) and toluene (5 mL) was heated to 80° C. for 18 hours. Removal of solvent in vacuo provided an oil, which was purified by silica gel chromatography (Gradient: 0% to 100% ethyl acetate in heptane), providing the product as a gray solid. Yield: 26.8 mg, 0.0886 mmol, 81%. LCMS m/z 301.0 (M−1). ¹H NMR (500 MHz, CDCl₃) δ 6.93 (d, J=6.4 Hz, 1H), 6.71 (d, J=6.6 Hz, 1H), 4.31-4.39 (m, 2H), 4.02 (dt, J=14.3, 4.4 Hz, 1H), 3.10 (ddd, J=14.5, 10.1, 5.5 Hz, 1H), 1.99-2.06 (m, 1H), 1.89-1.94 (m, 2H), 1.79 (dd, J=13.8, 5.2 Hz, 1H), 1.48 (s, 9H), 1.24 (d, J=7.1 Hz, 3H).

### Preparation 3: (5R,7S)-1-(3-Fluorophenyl)-7-methyl-2-thia-1,8-diazaspiro[4.5]decan-4-ol 2,2-dioxide (P3)

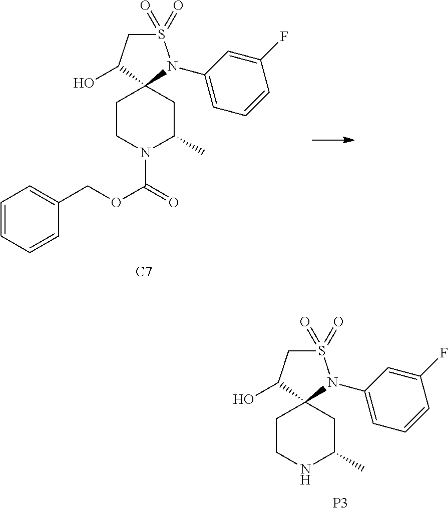

Palladium on carbon (10%, 38 mg) was added to a solution of benzyl (5R,7S)-1-(3-fluorophenyl)-4-hydroxy-7-methyl-2-thia-1,8-diazaspiro[4.5]decane-8-carboxylate 2,2-dioxide (C7) (118 mg, 0.263 mmol) in ethanol (5 mL), and the reaction mixture was hydrogenated at 50 psi for 18 hours. After filtration through Celite, rinsing with ethyl acetate and ethanol, the reaction was concentrated in vacuo to provide the product as a glass. By ¹H NMR analysis this material was a mixture of alcohol diastereomers. Yield: 84 mg, 0.27 mmol, quantitative. LCMS m/z 315.0 (M+1). ¹H NMR (400 MHz, CD₃OD), selected peaks, δ 7.49-7.55 (m, 1H), 7.39-7.44 (m, 2H), 7.25-7.30 (m, 1H), 4.40-4.46 (m, 1H), 3.85-3.92 (m, 1H), 3.44-3.50 (m, 1H), 2.30-2.45 (m, 2H), 2.03-2.12 (m, 1H), 1.84-1.92 (m, 1H), 1.17 and 1.26 (2 d, J=6.4 Hz, 3H).

### Preparation 4: (5R,7S)-1-(3-Fluorophenyl)-7-methyl-2-thia-1,8-diazaspiro[4.5]decane 2,2-dioxide (P4)

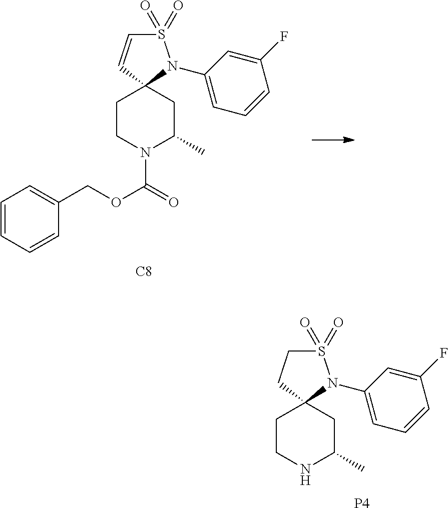

Palladium on carbon (10%, 35 mg) was added to a solution of benzyl (5R,7S)-1-(3-fluorophenyl)-7-methyl-2-thia-1,8-diazaspiro[4.5]dec-3-ene-8-carboxylate 2,2-dioxide (C8) (103 mg, 0.239 mmol) in ethanol, and the reaction mixture was hydrogenated at 50 psi for 4 hours. As the reaction was not complete, additional palladium on carbon was added, and hydrogenation was continued for 18 hours. After filtration through Celite, rinsing with ethyl acetate and ethanol, the reaction was concentrated in vacuo. Silica gel chromatography (Gradient: 0% to 20% [methanol containing 5% concentrated ammonium hydroxide] in dichloromethane) afforded the product as a gum. Yield: 43 mg, 0.144 mmol, 60%. LCMS m/z 299.1 (M+1). ¹H NMR (400 MHz, CDCl₃) δ 7.39 (ddd, J=9.0, 8.0, 6.4 Hz, 1H), 7.22 (ddd, J=7.9, 2, 1 Hz, 1H), 7.12-7.18 (m, 2H), 3.42 (dd, J=7.6, 7.4 Hz, 2H), 2.82 (ddd, J=12.8, 4.4, 4.4 Hz, 1H), 2.60-2.68 (m, 1H), 2.55 (ddd, J=12.8, 11.1, 3.0 Hz, 1H), 2.33-2.45 (m, 2H), 2.17-2.25 (m, 2H), 1.73 (ddd, J=14.0, 11.2, 4.8 Hz, 1H), 1.41 (dd, J=14.0, 10.0, 1H), 1.00 (d, J=6A Hz, 3H).

### Preparation 5: (5R,7S)-1-(3-Fluorophenyl)-N,7-dimethyl-2-thia-1,8-diazaspiro[4.5]dec-3-en-4-amine 2,2-dioxide (P5)

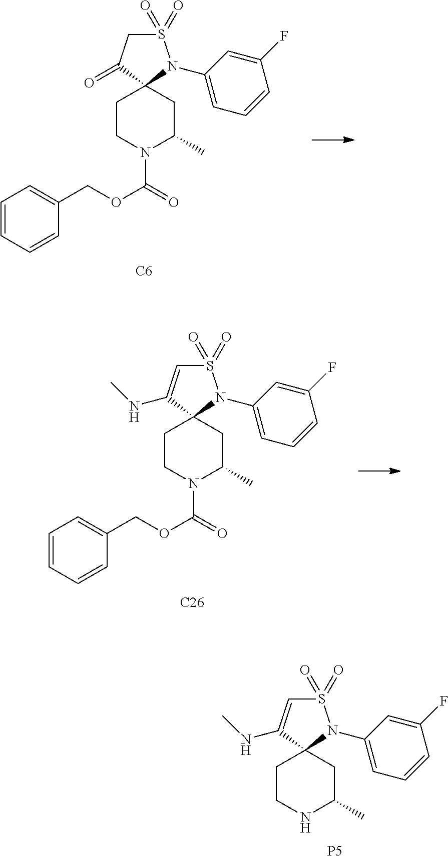

### Step 1. Synthesis of benzyl (5R,7S)-1-(3-fluorophenyl)-7-methyl-4-(methylamino)-2-thia-1,8-diazaspiro[4.5]dec-3-ene-8-carboxylate 2,2-dioxide (C26)

Methylamine (2 M in methanol, 0.116 mL, 0.232 mmol) and acetic acid (7.0 μL, 0.12 mmol) were added to a solution of benzyl (5R,7S)-1-(3-fluorophenyl)-7-methyl-4-oxo-2-thia-1,8-diazaspiro[4.5]decane-8-carboxylate 2,2-dioxide (C6) (52 mg, 0.12 mmol) in 1,2-dichloroethane (1.2 mL). After 20 minutes, the reaction mixture was treated with sodium triacetoxyborohydride (49.2 mg, 0.232 mmol), and the reaction was allowed to stir for 12 days. Additional methylamine solution (0.25 mL, 0.50 mmol) was added, and stirring was continued for 18 hours. The reaction was poured into ethyl acetate, washed with water, washed with saturated aqueous sodium chloride solution and dried over sodium sulfate. After the drying agent was filtered off, concentration in vacuo provided crude product (55 mg), which was taken into the next step without purification. LCMS m/z 460.1 (M+1). ¹H NMR (400 MHz, CDCl₃), characteristic peak: δ 2.74 (d, J=4.7 Hz, 3H).

### Step 2. Synthesis of (5R,7S)-1-(3-fluorophenyl)-N,7-dimethyl-2-thia-1,8-diazaspiro[4.5]dec-3-en-4-amine 2,2-dioxide (P5)

A solution of benzyl (5R,7S)-1-(3-fluorophenyl)-7-methyl-4-(methylamino)-2-thia-1,8-diazaspiro[4.5]dec-3-ene-8-carboxylate 2,2-dioxide (C26) (material from the previous step) in ethanol (5 mL) was hydrogenated using an H-Cube® continuous flow reactor (ThalesNano) (40° C., 10% Pd/C, 1 atmosphere H₂). The crude product was used without purification. LCMS m/z 326.1 (M+1).

### Preparation 6: (5R,7S)-1-(3-Fluorophenyl)-4-methoxy-7-methyl-2-thia-1,8-diazaspiro[4.5]dec-3-ene 2,2-dioxide (P6)

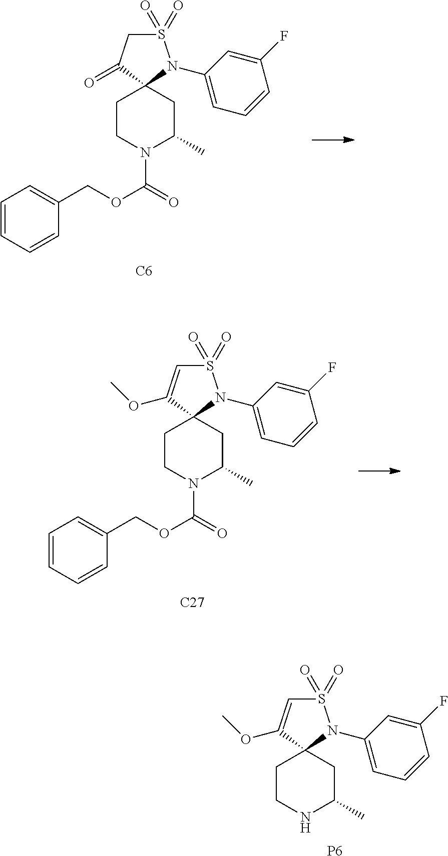

### Step 1. Synthesis of benzyl (5R,7S)-1-(3-fluorophenyl)-4-methoxy-7-methyl-2-thia-1,8-diazaspiro[4.5]dec-3-ene-8-carboxylate 2,2-dioxide (C27)

A slurry of benzyl (5R,7S)-1-(3-fluorophenyl)-7-methyl-4-oxo-2-thia-1,8-diazaspiro[4.5]decane-8-carboxylate 2,2-dioxide (C6) (48 mg, 0.11 mmol), dimethyl sulfate (0.051 mL, 0.54 mmol) and anhydrous potassium carbonate (37.3 mg, 0.270 mmol) in acetone (1 mL) was heated at 56° C. for 1 hour, then quenched with saturated aqueous sodium chloride solution. The mixture was extracted with ethyl acetate, and the organic layer was dried over sodium sulfate, filtered and concentrated in vacuo to provide the product as a solid (54 mg). This material was taken directly into the next step. LCMS m/z 461.0 (M+1). ¹H NMR (500 MHz, CDCl₃) δ 7.31-7.39 (m, 5H), 7.24-7.27 (m, 2H), 7.21 (ddd, J=9.5, 2, 2 Hz, 1H), 7.11-7.16 (m, 1H), 5.84 (s, 1H), 4.93 (AB quartet, upfield signals are broadened, J_{AB}=12.3 Hz, Δν_{AB}=55.5 Hz, 2H), 3.92-4.0 (m, 2H), 3.90 (s, 3H), 3.08-3.15 (m, 1H), 2.08-2.20 (m, 3H), 2.01 (dd, J=14.7, 7.7 Hz, 1H), 1.14 (d, J=6.6 Hz, 3H).

### Step 2. Synthesis of (5R,7S)-1-(3-fluorophenyl)-4-methoxy-7-methyl-2-thia-1,8-diazaspiro[4.5]dec-3-ene 2,2-dioxide (P6)

Compound P6 was prepared from benzyl (5R,7S)-1-(3-fluorophenyl)-4-methoxy-7-methyl-2-thia-1,8-diazaspiro[4.5]dec-3-ene-8-carboxylate 2,2-dioxide (C27) according to the general procedure for the synthesis of (5R,7S)-1-(3-fluorophenyl)-7-methyl-2-thia-1,8-diazaspiro[4.5]decan-4-ol 2,2-dioxide (P3) in Preparation 3, except that the crude product was purified by chromatography on silica gel (Gradient: 0% to 20% [methanol containing 5% concentrated ammonium hydroxide] in dichloromethane). The product was obtained as a solid. Yield: 25 mg, 0.077 mmol, 70% over 2 steps. LCMS m/z 327.5 (M+1). ¹H NMR (500 MHz, CDCl₃) δ 7.42 (ddd, J=8.1, 8.1, 6.4 Hz, 1H), 7.32 (ddd, J=7.9, 1.7, 1.0 Hz, 1H), 7.24 (ddd, J=9.4, 2.2, 2.2 Hz, 1H), 7.19 (dddd, J=8.3, 8.3, 2.6, 1.0 Hz, 1H), 5.83 (s, 1H), 3.86 (s, 3H), 2.84 (ddd, J=12.8, 5.4, 1.9 Hz, 1H), 2.44-2.53 (m, 2H), 2.15 (ddd, J=14.5, 13.2, 5.5 Hz, 1H), 2.01-2.07 (m, 2H), 1.78 (dd, J=14.4, 12.1 Hz, 1H), 0.96 (d, J=6.2 Hz, 3H).

### Preparation 7: (5R,7S)-1-(3-Fluorophenyl)-3.3.7-trimethyl-2-thia-1,8-diazaspiro[4.5]decan-4-one 2,2-dioxide (P7)

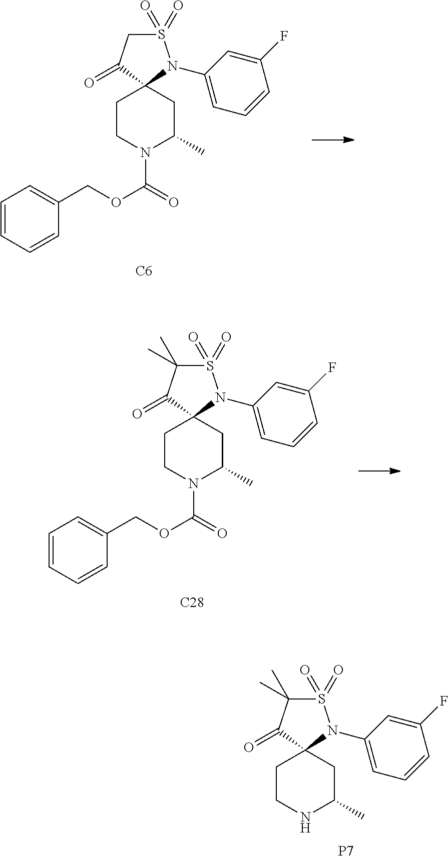

### Step 1. Synthesis of benzyl (5R,7S)-1-(3-fluorophenyl)-3.3.7-trimethyl-4-oxo-2-thia-1,8-diazaspiro[4.5]decane-8-carboxylate 2,2-dioxide (C28)

A slurry of benzyl (5R,7S)-1-(3-fluorophenyl)-7-methyl-4-oxo-2-thia-1,8-diazaspiro[4.5]decane-8-carboxylate 2,2-dioxide (C6) (31 mg, 0.069 mmol) and potassium carbonate (28.6 mg, 0.207 mmol) in N,N-dimethylformamide (0.35 mL) was cooled to 0° C. and treated with a solution of iodomethane (7.0 μL, 0.11 mmol) in dichloromethane (63 μL). After 18 hours at room temperature, the reaction was treated with additional iodomethane (0.5 equivalents) and stirred for an additional 2 hours, at which time it was poured into ethyl acetate and washed three times with water and once with saturated aqueous sodium chloride solution. The organic layer was dried over sodium sulfate, filtered and concentrated under reduced pressure. The residue was combined with material from a similar reaction run on 33 mg (0.074 mmol) of substrate, using cesium carbonate as base. Purification via silica gel chromatography (Gradient: 0% to 100% ethyl acetate in heptane) afforded the product as a glass. Yield: 48 mg, 0.10 mmol, 70%. LCMS m/z 475.0 (M+1). ¹H NMR (400 MHz, CDCl₃) δ 7.42 (ddd, J=8.2, 8.1, 6.3 Hz, 1H), 7.26-7.37 (m, 5H), 7.16-7.23 (m, 2H), 7.14 (ddd, J=9.1, 2.2 Hz, 1H), 5.01 (AB quartet, upfield peaks are broadened, J_{AB}=12.3 Hz, Δν_{AB}=24.7 Hz, 2H), 4.18-4.26 (m, 1H), 3.99-4.07 (m, 1H), 3.37-3.46 (m, 1H), 2.01-2.11 (m, 3H), 1.80-1.89 (m, 1H), 1.66 (s, 3H), 1.64 (s, 3H), 1.23 (d, J=7.0 Hz, 3H).

### Step 2. Synthesis of (5R,7S)-1-(3-fluorophenyl)-3,3,7-trimethyl-2-thia-1,8-diazaspiro[4.5]decan-4-one 2,2-dioxide (P7)

A solution of benzyl (5R,7S)-1-(3-fluorophenyl)-3,3,7-trimethyl-4-oxo-2-thia-1,8-diazaspiro[4.5]decane-8-carboxylate 2,2-dioxide (C28) (48 mg, 0.10 mmol) in methanol was hydrogenated using an H-Cube® continuous flow reactor (ThalesNano) (45° C., 10% Pd/C, 1 atmosphere H₂). Only partial reduction was effected, so the hydrogenation was repeated. The crude product, obtained as a gum, was used without purification. Yield: 30 mg, 0.088 mmol, 88%. LCMS m/z 341.0 (M+1). ¹H NMR (500 MHz, CDCl₃) δ 7.48 (ddd, J=8.2, 8.2, 6.4 Hz, 1H), 7.29-7.31 (m, 1H), 7.24 (br ddd, J=8, 8, 2 Hz, 1H), 7.20 (ddd, J=8.8, 2, 2 Hz, 1H), 3.09 (ddd, J=13.0, 4.7, 2.6 Hz, 1H), 2.61-2.69 (m, 1H), 2.49 (ddd, J=13.1, 13.1, 3.1 Hz, 1H), 2.37 (ddd, J=14.9, 13.1, 5.0 Hz, 1H), 2.24-2.30 (m, 2H), 2.04-2.10 (m, 1H), 1.61 (s, 3H), 1.61 (s, 3H), 1.18 (d, J=6.4 Hz, 3H).

### Preparation 8: 4-Hydroxy-3-isopropoxybenzaldehyde (P8)

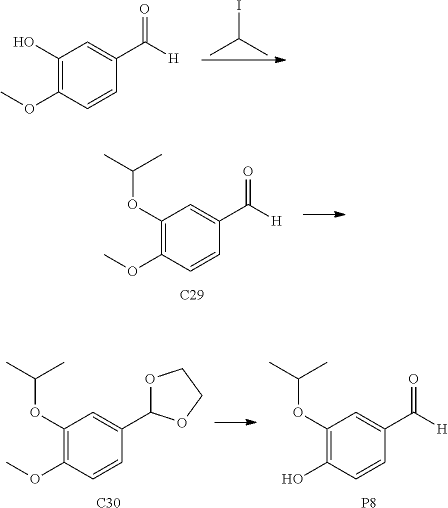

### Step 1. Synthesis of 3-isopropoxy-4-methoxybenzaldehyde (C29)

A solution of 3-hydroxy-4-methoxybenzaldehyde (5.00 g, 32.9 mmol) in N,N-dimethylformamide (100 mL) was treated with potassium carbonate (9.08 g, 65.7 mmol) and 2-iodopropane (6.57 mL, 65.7 mmol). The reaction was stirred for 4 hours and then additional 2-iodopropane (3.29 mL, 32.9 mmol) was added and the mixture was allowed to react for an additional hour. It was then poured into water and extracted with ethyl acetate (3×20 mL). The combined organic layers were washed with 1 N aqueous sodium hydroxide solution, then with saturated aqueous sodium chloride solution, dried, filtered and concentrated in vacuo to provide the product as an oil. Yield: 4.60 g, 23.7 mmol, 72%. LCMS m/z 195.2 (M+1). ¹H NMR (400 MHz, CDCl₃) δ 9.85 (s, 1H), 7.42-7.46 (m, 2H), 6.99 (d, J=8.1 Hz, 1H), 4.65 (m, 1H), 3.95 (s, 3H), 1.41 (d, J=6.2 Hz, 6H).

### Step 2. Synthesis of 2-(3-isopropoxy-4-methoxyphenyl)-1,3-dioxolane (C30)

Ethylene glycol (99%, 2.63 mL, 47.4 mmol) and para-toluenesulfonic acid monohydrate (97%, 75 mg, 0.38 mmol) were added to a solution of 3-isopropoxy-4-methoxybenzaldehyde (C29) (4.6 g, 23.7 mmol) in toluene (79 mL). The reaction flask was equipped with a Dean-Stark trap, and the contents were heated at reflux for 5 hours. The reaction was poured into aqueous potassium carbonate solution, and the organic layer was then washed an additional two times with aqueous potassium carbonate solution, and once with saturated aqueous sodium chloride solution. The organic layer was dried, filtered and concentrated in vacuo; NMR and LCMS revealed that the reaction was incomplete, so the product was resubjected to the reaction conditions, heating at reflux for 18 hours. The workup was repeated, to afford the product as an oil. Yield: 5.0 g, 21.0 mmol, 89%. ¹H NMR (400 MHz, CDCl₃) δ 7.03 (m, 2H), 6.88 (d, J=8.7 Hz, 1H), 5.75 (s, 1H), 4.57 (septet, J=6.0 Hz, 1H), 4.14 (m, 2H), 4.02 (m, 2H), 3.86 (s, 3H), 1.38 (d, J=6.2 Hz, 6H).

### Step 3. Synthesis of 4-hydroxy-3-isopropoxybenzaldehyde (P8)

Lithium wire (cut into small segments, 204 mg, 29.4 mmol) was added to a solution of chlorodiphenylphosphine (2.17 mL, 11.7 mmol) in tetrahydrofuran (18.7 mL), and the reaction was stirred for 1 hour. A solution of 2-(3-isopropoxy-4-methoxyphenyl)-1,3-dioxolane (C30) (2.00 g, 8.39 mmol) in tetrahydrofuran (5 mL) was then added drop-wise to the dark red mixture, and the reaction was stirred for 2 hours. It was then filtered into an aqueous sodium hydroxide solution, and extracted with diethyl ether (3×15 mL); the combined organic layers were washed with 1 N aqueous sodium hydroxide solution, and the aqueous layers were combined and cooled in an ice bath. This aqueous phase was acidified with concentrated aqueous hydrochloric acid. The mixture was extracted with diethyl ether (3×10 mL) and these three organic layers were combined and washed with saturated aqueous sodium chloride solution, dried and concentrated in vacuo to give the product as an oil. Yield: 740 mg, 4.11 mmol, 49%. ¹H NMR (400 MHz, CDCl₃) δ 9.82 (s, 1H), 7.40 (m, 2H), 7.05 (d, J=8.0 Hz, 1H), 6.30 (s, 1H), 4.73 (septet, J=6.1 Hz, 1H), 1.41 (d, J=6.0 Hz, 6H).

### Preparation 9: (5R,7S)-1-(3,4-Difluorophenyl-7-methyl-2-thia-1,8-diazaspiro[4.5]dec-3-ene 2,2-dioxide (P9)

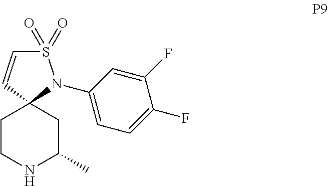 [SMILES: C[C@H]1C[C@@]2(C=CS(=O)(=O)N2c2ccc(F)c(F)c2)CCN1]

(5R,7S)-1-(3,4-Difluorophenyl)-7-methyl-2-thia-1,8-diazaspiro[4.5]dec-3-ene 2,2-dioxide (P9) was prepared in analogous manner to (5R,7S)-1-(3-fluorophenyl)-7-methyl-2-thia-1,8-diazaspiro[4.5]dec-3-ene 2,2-dioxide (P1) in Preparation 1, except that 3,4-difluoroaniline was employed in place of 3-fluoroaniline. LCMS m/z 315.2 (M+1). ¹H NMR (400 MHz, CDCl₃) δ 7.23-7.36 (m, 3H), 6.77 (AB quartet, J_{AB}=7.1 Hz, Δν_{AB}=18.0 Hz, 2H), 2.89 (ddd, J=12.7, 5.1, 3.5 Hz, 1H), 2.61-2.70 (m, 1H), 2.56 (ddd, J=12.7, 11.7, 3.2 Hz, 1H), 2.03-2.10 (m, 2H), 1.95 (ddd, J=14.3, 11.7, 5.1 Hz, 1H), 1.61 (dd, J=14.4, 10.8 Hz, 1H), 1.03 (d, J=6.2 Hz, 3H)

### Preparation 10: tert-Butyl (5R,7S)-7-methyl-1-pyridin-2-yl-2-thia-1,8-diazaspiro[4.5]decane-8-carboxylate 2,2-dioxide (P10)

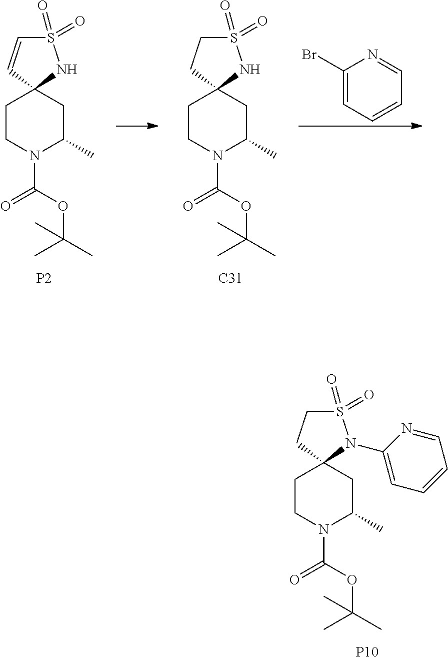

### Step 1. Synthesis of tert-butyl (5R,7S)-7-methyl-2-thia-1,8-diazaspiro[4.5]decane-8-carboxylate 2,2-dioxide (C31)

tert-Butyl (5R,7S)-7-methyl-2-thia-1,8-diazaspiro[4.5]dec-3-ene-8-carboxylate 2,2-dioxide (P2) was converted to the product using the method described in Preparation 4 for hydrogenation of benzyl (5R,7S)-1-(3-fluorophenyl)-7-methyl-2-thia-1,8-diazaspiro[4.5]dec-3-ene-8-carboxylate 2,2-dioxide (C8). In this case, chromatographic purification was not required. The product was obtained as a white solid. Yield: 306 mg, 1.01 mmol, 98%. APCI m/z 303.3 (M−1). ¹H NMR (500 MHz, CDCl₃) δ 1.21 (d, J=7.1 Hz, 3H), 1.47 (s, 9H), 1.72-1.79 (m, 2H), 1.85-1.90 (m, 1H), 1.99 (dd, J=13.8, 6.5 Hz, 1H), 2.39-2.51 (m, 2H), 3.02 (ddd, J=14.4, 11.8, 3.7 Hz, 1H), 3.16-3.28 (m, 2H), 3.98-4.04 (m, 2H), 4.31-4.38 (m, 1H).

### Step 2. Synthesis of tert-butyl (5R,7S)-7-methyl-1-pyridin-2-yl-2-thia-1,8-diazaspiro[4.5]decane-8-carboxylate 2,2-dioxide (P10)

tert-Butyl (5R,7S)-7-methyl-2-thia-1,8-diazaspiro[4.5]decane-8-carboxylate 2,2-dioxide (C31) (100 mg, 0.329 mmol), copper(I) iodide (251 mg, 1.32 mmol) and potassium phosphate (210 mg, 0.989 mmol) were combined in a sealed vial, and the vial was evacuated and flushed with argon three times. A solution of 2-bromopyridine (35 μL, 0.36 mmol) and N,N′-dimethylethylenediamine (99%, 178 μL, 1.64 mmol) in N,N-dimethylformamide (6 mL) was added to the reaction vial, and the reaction mixture was placed on a plate stirrer at 110° C. for 72 hours. The reaction was then cooled to room temperature and mixed with water (150 mL); the resulting mixture was extracted with diethyl ether (3×25 mL), and the combined organic layers were washed with water (50 mL), washed with saturated aqueous sodium chloride solution (50 mL), and dried over magnesium sulfate. Filtration and removal of solvent under reduced pressure provided a residue, which was purified using silica gel chromatography (Gradient: 20% to 50% ethyl acetate in heptane), to provide the product as an oil containing a minor impurity. Yield: 11.2 mg, 0.0294 mmol, 9%. LCMS m/z 382.1 (M+1). ¹H NMR (500 MHz, CDCl₃) δ 1.24 (d, J=7.2 Hz, 3H), 1.46 (s, 9H), 1.64 (ddd, J=13.7, 1.9, 1.8 Hz, 1H), 1.78-1.83 (m, 1H), 2.56-2.64 (m, 2H), 2.69 (ddd, J=13.1, 5.7, 5.7 Hz, 1H), 2.87 (br ddd, J=13, 13, 5 Hz, 1H), 3.00 (br dd, J=14, 14 Hz, 1H), 3.36-3.39 (m, 2H), 4.06-4.13 (m, 1H), 4.43-4.52 (m, 1H), 7.15 (ddd, J=7.4, 4.9, 1.0 Hz, 1H), 7.47 (br d, J=8.2 Hz, 1H), 7.70 (ddd, J=8.2, 7.4, 2.1 Hz, 1H), 8.45 (br dd, J=4.8, 1.9 Hz, 1H).

### Preparation 11: (5R,7S)-7-Methyl-1-pyrazin-2-yl-2-thia-1,8-diazaspiro[4.5]decane 2,2-dioxide (P11)

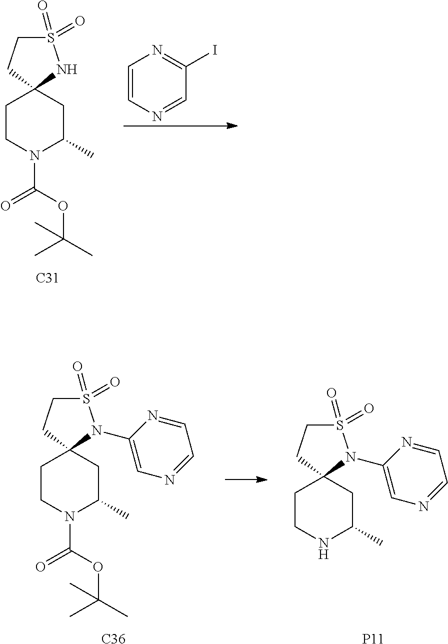

### Step 1. Synthesis of tert-butyl (5R,7S)-7-methyl-1-(pyrazin-2-yl)-2-thia-1,8-diazaspiro[4.5]decane-8-carboxylate 2,2-dioxide (C36)

A sealed tube was charged with copper(I) iodide (0.047 g, 0.246 mmol), potassium carbonate (0.459 g, 3.29 mmol) and tert-butyl (5R,7S)-7-methyl-2-thia-1,8-diazaspiro[4.5]decane-8-carboxylate 2,2-dioxide (C31) (0.500 g, 1.64 mmol). N,N-Dimethylformamide (11 mL) was added, followed by trans-N,N′-dimethylcyclohexane-1,2-diamine (0.52 mL, 3.3 mmol) and 2-iodopyrazine (0.162 mL, 1.64 mmol). The resulting blue suspension was stirred at room temperature for 5 minutes, then heated to 100° C. for 16 hours. The reaction mixture was cooled to room temperature and partitioned between ethyl acetate (100 mL) and aqueous ammonium chloride solution (10%, 200 mL). The aqueous phase was extracted with ethyl acetate (3×50 mL) and the combined organic layers were washed with water (3×100 mL), with saturated aqueous sodium chloride solution (100 mL) and dried over magnesium sulfate. Filtration and concentration in vacuo provided a residue, which was purified via silica gel chromatography (Gradient: 40% to 50% ethyl acetate in heptane) to afford the product as an oil. Yield: 0.268 g, 0.701 mmol, 43%. LCMS m/z 383.1 (M+1). ¹H NMR (400 MHz, CDCl₃) δ 8.80 (d, J=1.4 Hz, 1H), 8.37-8.40 (m, 2H), 4.46-4.57 (m, 1H), 4.07-4.17 (m, 1H), 3.40-3.45 (m, 2H), 3.01 (br dd, J=14, 14 Hz, 1H), 2.87 (ddd, J=13, 13, 4 Hz, 1H), 2.56-2.78 (m, 3H), 1.77-1.83 (m, 1H), 1.64 (ddd, J=14, 2, 2 Hz, 1H), 1.47 (s, 9H), 1.26 (d, J=7.2 Hz, 3H).

### Step 2. Synthesis of (5R,7S)-7-methyl-1-pyrazin-2-yl-2-thia-1,8-diazaspiro[4.5]decane 2,2-dioxide (P11)

Trifluoroacetic acid (0.39 mL, 5.0 mmol) and triethylsilane (0.155 mL, 0.968 mmol) were added to a solution of tert-butyl (5R,7S)-7-methyl-1-(pyrazin-2-yl)-2-thia-1,8-diazaspiro[4.5]decane-8-carboxylate 2,2-dioxide (C36) (148 mg, 0.387 mmol) in dichloromethane (5 mL), and the reaction was allowed to stir for 18 hours. Water (100 mL) was added, and the aqueous layer was washed with dichloromethane (2×20 mL). The aqueous layer was then basified to pH 12 with an aqueous sodium hydroxide solution (1 M, 15 mL). After extraction with dichloromethane (3×25 mL), the combined organic extracts were washed with saturated aqueous sodium chloride solution, dried over magnesium sulfate, filtered and concentrated in vacuo. The product was obtained as a yellow oil. Yield: 84 mg, 0.30 mmol, 78%. LCMS m/z 283.2 (M+1). ¹H NMR (400 MHz, CDCl₃) δ 8.73 (d, J=1.4 Hz, 1H), 8.55 (d, J=2.5 Hz, 1H), 8.52 (dd, J=2.5, 1.4 Hz, 1H), 3.49 (dd, J=7.6, 7.6 Hz, 2H), 2.84-2.90 (m, 1H), 2.46-2.67 (m, 6H), 1.63 (ddd, J=14, 11, 4 Hz, 1H), 1.32 (dd, J=13.9, 10.2 Hz, 1H), 0.99 (d, J=6.2 Hz, 3H).

### EXAMPLES

### Example 1

### 4-{[(5R,7S)-1-(3-Fluorophenyl)-7-methyl-2,2-dioxido-2-thia-1,8-diazaspiro[4.5]dec-8-yl]methyl}-2-isopropoxyphenol (1)

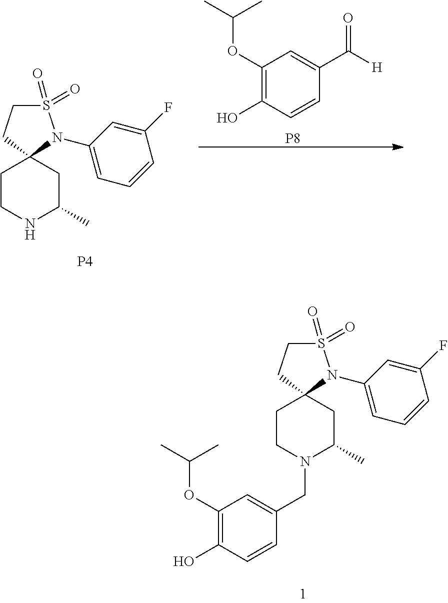

4-Hydroxy-3-isopropoxybenzaldehyde (P8) (58.9 mg, 0.327 mmol), 4 Å molecular sieves and acetic acid (12 μL, 0.21 mmol) were added to a solution of (5R,7S)-1-(3-fluorophenyl)-7-methyl-2-thia-1,8-diazaspiro[4.5]decane 2,2-dioxide (P4) (64.0 mg, 0.214 mmol) in 1,2-dichloroethane (1 mL), and the mixture was stirred for 18 hours at room temperature. Sodium triacetoxyborohydride (92.4 mg, 0.436 mmol) was added, and the reaction was continued for an additional 24 hours, then poured into aqueous sodium bicarbonate solution. After two extractions with ethyl acetate, the combined organic layers were dried over sodium sulfate, filtered and concentrated in vacuo. Two chromatographic purifications on silica gel (First gradient: 0% to 10% methanol in dichloromethane; Second gradient: 0% to 10% methanol in ethyl acetate) provided the product as a colorless foam. Yield: 96.0 mg, 0.208 mmol, 97%. LCMS m/z 463.1 (M+1). ¹H NMR (400 MHz, CDCl₃) δ 7.37 (ddd, J=8.2, 8.1, 6.4 Hz, 1H), 7.13-7.18 (m, 2H), 7.08 (ddd, J=9.4, 2.2, 2.2 Hz, 1H), 6.80 (d, J=8.0 Hz, 1H), 6.73 (br d, J=1.8 Hz, 1H), 6.64 (br dd, J=8.0, 1.8 Hz, 1H), 5.61 (br s, 1H), 4.50 (septet, J=6.1 Hz, 1H), 3.40 (AB quartet, J_{AB}=13.3 Hz, Δν_{AB}=79.2 Hz, 2H), 3.32-3.37 (m, 2H), 2.77-2.85 (m, 1H), 2.44-2.59 (m, 3H), 2.28-2.35 (m, 1H), 2.02 (dd, J=13.4, 5.2 Hz, 1H), 1.94 (br ddd, J=13, 9, 4 Hz, 1H), 1.70-1.77 (m, 1H), 1.66 (br ddd, J=13, 5, 2 Hz, 1H), 1.32 (d, J=6.1 Hz, 6H), 1.09 (d, J=6.8 Hz, 3H).

### Example 2

### (5R,7S)-1-(3-Fluorophenyl)-8-[(4-isobutyl-1,3-oxazol-5-yl)methyl]-3,7-dimethyl-2-thia-1,8-diazaspiro[4.5]decane 2,2-dioxide, formate salt (2)

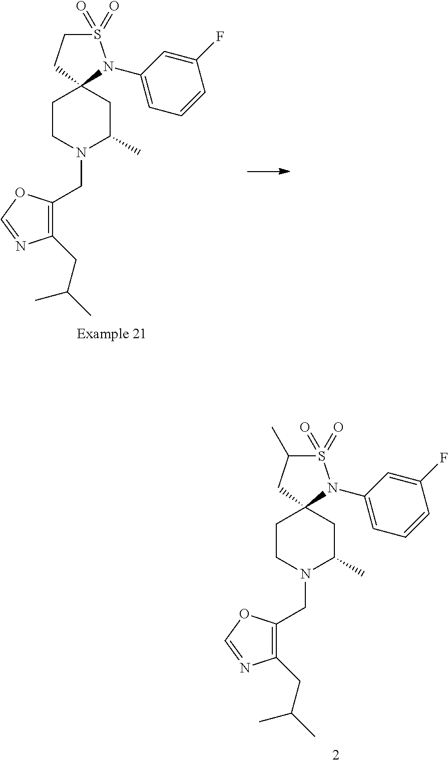

A solution of lithium diisopropylamide (1.8 M in heptane/tetrahydrofuran/ethylbenzene, 0.157 mL, 0.28 mmol) was added drop-wise over 5 minutes to a solution of (5R,7S)-1-(3-fluorophenyl)-8-[(4-isobutyl-1,3-oxazol-5-yl)methyl]-7-methyl-2-thia-1,8-diazaspiro[4.5]decane 2,2-dioxide (Example 21) (41 mg, 0.094 mmol) in tetrahydrofuran (0.5 mL) at −78° C., and the solution was stirred for 1 hour at that temperature. A solution of iodomethane (18.0 μL, 0.288 mmol) in tetrahydrofuran (0.3 mL) was added via syringe, and the reaction was monitored until the product was visible by LCMS analysis. The reaction was quenched with saturated aqueous ammonium chloride solution; after warming to room temperature, it was poured into water and extracted with ethyl acetate. The combined organic layers were washed with saturated aqueous sodium chloride solution, dried over sodium sulfate, filtered and concentrated in vacuo. Purification via reversed-phase chromatography (Column: Phenomenex Luna C18(2), 5 μm; Mobile phase A: 0.1% formic acid in water (v/v); Mobile phase B: 0.1% formic acid in methanol (v/v); Gradient: 5% to 100% B) provided the product as a gum. By ¹H NMR, the product was a roughly 2:1 mixture of diastereomers at the newly introduced methyl group. Yield: 26 mg, 0.052 mmol, 55%. LCMS m/z 450.2 (M+1). ¹H NMR (500 MHz, CDCl₃) δ 8.23 (s, 1H), 7.72 and 7.75 (2 s, 1H), 7.27-7.33 (m, 1H), 7.08-7.15 (m, 2H), 7.03-7.06 (m, 1H), 3.75 (AB quartet, J_{AB}=15.2 Hz, Δν_{AB}=30.0 Hz) and 3.83 (AB quartet, J_{AB}=15.5 Hz, Δν_{AB}=59.5 Hz, total 2H), 3.44-3.56 (m, 1H), 2.37-2.84 (m, 4H), 1.83-2.30 (m, 8H), 1.49 (d, J=6.6 Hz, 3H), 1.18 (d, J=6.5 Hz) and 1.32 (d, J=6.4 Hz, total 3H), 0.89 (d, J=6.7 Hz) plus 0.84 (d, J=6.6 Hz) and 0.86 (d, J=6.8 Hz) plus 0.82 (d, J=6.7 Hz, total 6H).

### Example 3

### 6-{[(5R,7S)-1-(3-Fluorophenyl)-7-methyl-2,2-dioxido-2-thia-1,8-diazaspiro[4.5]dec-8-yl]methyl}-4-isopropoxypyridin-3-ol (3)

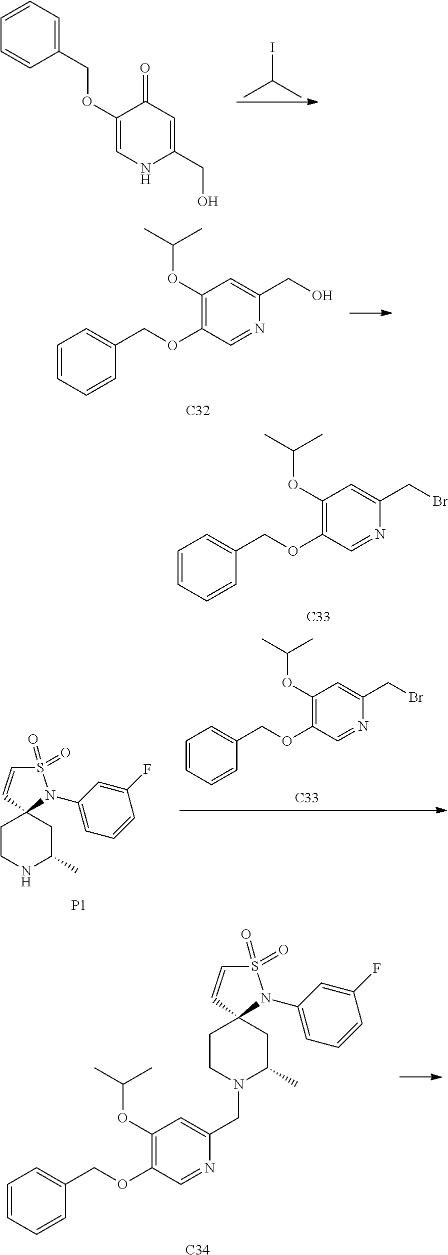

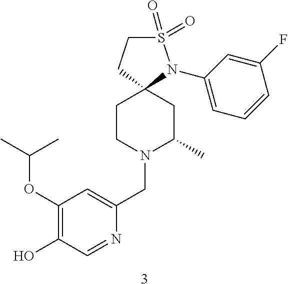 [SMILES: CC(C)Oc1cc(CN2CC[C@]3(CCS(=O)(=O)N3c3cccc(F)c3)C[C@@H]2C)ncc1O ; "3"]

### Step 1. Synthesis of 5-(benzyloxy)-2-(bromomethyl)-4-isopropoxypyridine (C33)

A. Synthesis of [5-(benzyloxy)-4-isopropoxypyridin-2-yl]methanol (C32). A solution of 5-(benzyloxy)-2-(hydroxymethyl)pyridin-4(1H)-one (prepared by a procedure similar to that reported by M. M. O'Malley et al., Organic Letters 2006, 8, 2651-2652) (1.3 g, 5.6 mmol) in N,N-dimethylformamide (11.2 mL) was treated with 2-iodopropane (95%, 1.77 mL, 16.8 mmol) and potassium carbonate (1.17 g, 8.42 mmol). The slurry was stirred for 2.5 hours at room temperature and then heated at 80° C. for 18 hours, with addition of 2-iodopropane (1.3 mL, 12 mmol) after the first hour. The mixture was extracted three times with ethyl acetate, and the combined organic layers were dried over sodium sulfate. Filtration and removal of solvent in vacuo gave a residue, which was purified by silica gel chromatography (Eluants: 0%, then 20%, then 40% 2-propanol in ethyl acetate) to provide the product as a brown solid. Yield: 700 mg, 2.56 mmol, 46%. ¹H NMR (400 MHz, DMSO-d₆) δ 8.06 (s, 1H), 7.30-7.44 (m, 5H), 7.06 (s, 1H), 5.28 (t, J=5.9 Hz, 1H), 5.13 (s, 2H), 4.72 (septet, J=6.0 Hz, 1H), 4.42 (d, J=5.9 Hz, 2H), 1.32 (d, J=6.0 Hz, 6H).

B. Synthesis of 5-(benzyloxy)-2-(bromomethyl)-4-isopropoxypyridine (C33). Phosphorus tribromide (0.104 mL, 1.10 mmol) was added to a solution of [5-(benzyloxy)-4-isopropoxypyridin-2-yl]methanol (C32) (100 mg, 0.366 mmol) in dichloromethane (1.46 mL) at 0° C. After 1.5 hours at room temperature, the reaction mixture was carefully quenched with saturated aqueous sodium bicarbonate solution and diluted with ethyl acetate. The aqueous layer was extracted with ethyl acetate, and the combined organic layers were washed with saturated aqueous sodium chloride solution, dried over sodium sulfate, filtered and concentrated in vacuo to provide the product as an oil. Yield: 115 mg, 0.342 mmol, 93%. ¹H NMR (400 MHz, CDCl₃) δ 8.09 (s, 1H), 7.30-7.44 (m, 5H), 6.95 (s, 1H), 5.16 (s, 2H), 4.69 (septet, J=6.1 Hz, 1H), 4.48 (s, 2H), 1.43 (d, J=6.1 Hz, 6H).

### Step 2. Synthesis of (5R,7S)-8-{[5-(benzyloxy)-4-isopropoxypyridin-2-yl]methyl}-1-(3-fluorophenyl)-7-methyl-2-thia-1,8-diazaspiro[4.5]dec-3-ene 2,2-dioxide (C34)

Cesium carbonate (99%, 111 mg, 0.337 mmol) was added to a solution of (5R,7S)-1-(3-fluorophenyl)-7-methyl-2-thia-1,8-diazaspiro[4.5]dec-3-ene 2,2-dioxide (P1) (50 mg, 0.17 mmol) and 5-(benzyloxy)-2-(bromomethyl)-4-isopropoxypyridine (C33) (85.4 mg, 0.254 mmol) in N,N-dimethylformamide (0.85 mL), and the mixture was stirred for 18 hours. It was then diluted with water (4.25 mL) and extracted with ethyl acetate. The combined organic layers were dried over sodium sulfate, filtered and concentrated under reduced pressure. Purification via silica gel chromatography (Gradient: 0% to 60% [1:1-methanol:dichloromethane] in dichloromethane) provided the product as a gum. Yield: 70 mg, 0.13 mmol, 76%. ¹H NMR (400 MHz, CDCl₃) δ 8.02 (s, 1H), 7.28-7.43 (m, 6H), 7.16-7.21 (m, 2H), 7.13 (br ddd, J=9, 2, 2 Hz, 1H), 7.09 (d, J=7.2 Hz, 1H), 6.80 (d, J=7.2 Hz, 1H), 6.78 (s, 1H), 5.11 (s, 2H), 4.57 (septet, J=6.1 Hz, 1H), 3.52 (AB quartet, J_{AB}=13.8 Hz, Δν_{AB}=121.7 Hz, 2H), 2.83-2.88 (m, 1H), 2.66 (ddd, 12.7, 8.7, 3.4 Hz, 1H), 2.34 (ddd, J=12.7, 6.8, 3.9 Hz, 1H), 2.16 (dd, J=13.7, 4.7 Hz, 1H), 2.00 (ddd, J=13.4, 8.7, 3.8 Hz, 1H), 1.82-1.87 (m, 1H), 1.76 (dd, J=13.8, 5.7 Hz, 1H), 1.34-1.36 (m, 6H), 1.13 (d, J=6.6 Hz, 3H).

### Step 3. Synthesis of 6-([(5R,7S)-1-(3-fluorophenyl)-7-methyl-2,2-dioxido-2-thia-1,8-diazaspiro[4.5]dec-8-yl]methyl)-4-isopropoxypyridin-3-ol (3)

A solution of (5R,7S)-8-{[5-(benzyloxy)-4-isopropoxypyridin-2-yl]methyl}-1-(3-fluorophenyl)-7-methyl-2-thia-1,8-diazaspiro[4.5]dec-3-ene 2,2-dioxide (C34) (35 mg, 0.063 mmol) in methanol was hydrogenated using an H-Cube® continuous flow reactor (ThalesNano) (50° C., 10% Pd/C, 1 atmosphere H2). The crude product was purified using silica gel chromatography (Gradient: 0% to 100% [1:1 methanokdichloromethane] in dichloromethane) to afford the product as a gum. Yield: 16 mg, 0.035 mmol, 56%. ¹H NMR (400 MHz, CDCl₃) δ 8.06 (s, 1H), 7.37 (ddd, J=8, 8, 6.4 Hz, 1H), 7.12-7.17 (m, 2H), 7.07 (ddd, J=9.4, 2, 2 Hz, 1H), 6.79 (s, 1H), 4.59 (septet, J=6.1 Hz, 1H), 3.55 (AB quartet, J_{AB}=13.8 Hz, Δν_{AB}=55.6 Hz, 2H), 3.31-3.39 (m, 2H), 2.84-2.92 (m, 1H), 2.64 (br ddd, J=13, 9, 3 Hz, 1H), 2.45-2.59 (m, 2H), 2.38 (br ddd, J=13, 6, 4 Hz, 1H), 2.06 (dd, J=13.5, 5.0 Hz, 1H), 1.96 (br ddd, J=13, 9, 4 Hz, 1H), 1.74-1.82 (m, 1H), 1.70 (br dd, J=13, 5 Hz, 1H), 1.34 (d, J=6.1 Hz, 6H), 1.13 (d, J=6.8 Hz, 3H).

### Example 4

### (5R,7S)-8-(3-Isopropoxybenzyl)-7-methyl-1-(6-methylpyridin-2-yl)-2-thia-1,8-diazaspiro[4.5]dec-3-ene 2,2-dioxide (4)

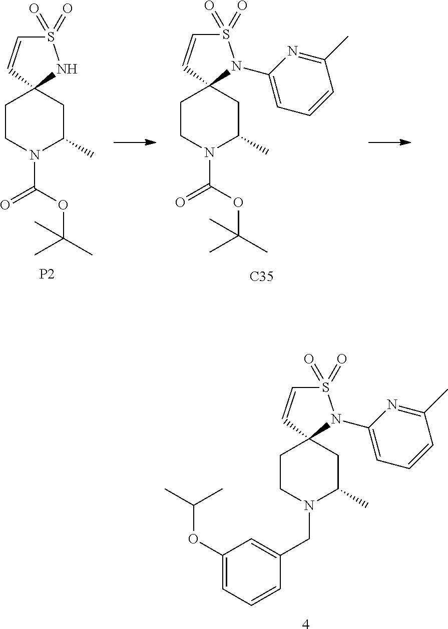

### Step 1. Synthesis of tert-butyl (5R,7S)-7-methyl-1-(6-methylpyridin-2-yl)-2-thia-1,8-diazaspiro[4.5]dec-3-ene-8-carboxylate 2,2-dioxide (C35)

Palladium acetate (1.8 mg, 0.0080 mmol) and 5-(di-tert-butylphosphino)-1′,3′,5′-triphenyl-1′H-1,4′-bipyrazole (8.1 mg, 0.016 mmol) were stirred in toluene (1 mL) at 20° C. for 30 min. To this solution was added tert-butyl (5R,7S)-7-methyl-2-thia-1,8-diazaspiro[4.5]dec-3-ene-8-carboxylate 2,2-dioxide (P2) (20 mg, 0.66 mmol), 2-bromo-6-methylpyridine (35 mg, 0.20 mmol) and cesium carbonate (12.3 mg, 0.205 mmol). The vessel was sealed and the resulting mixture was stirred at 110° C. for 20 hours. The reaction mixture was cooled and the product isolated by silica gel chromatography (Gradient: 25% to 75% ethyl acetate in heptane). The product was obtained as a colorless oil. Yield: 2.9 mg, 0.074 mmol, 11%. LCMS m/z 394.1 (M+1). ¹H NMR (400 MHz, CDCl₃) δ 7.56 (t, J=7.9 Hz, 1H), 7.47 (d, J=8.2 Hz, 1H), 7.37 (d, J=7.4 Hz, 1H), 6.92 (d, J=7.4 Hz, 1H), 6.74 (d, J=7.4 Hz, 1H), 4.62 (dd, J=8.2, 2.0 Hz, 1H), 3.18-3.32 (m, 3H), 2.99-3.09 (m, 1H), 2.46 (s, 3H), 1.71 (dd, J=13.6, 1.5 Hz, 1H), 1.60 (dt, J=13.9, 1.9 Hz, 1H), 1.46 (s, 9H), 1.26 (d, J=7.0 Hz, 3H).

### Step 2. Synthesis of (5R,7S)-8-(3-isopropoxybenzyl)-7-methyl-1-(6-methylpyridin-2-yl)-2-thia-1,8-diazaspiro[4.5]dec-3-ene 2,2-dioxide (4)

Hydrochloric acid (4.0 M in 1,4-dioxane, 50 μL, 0.20 mmol) was added to a 20 C solution of tert-butyl (5R,7S)-7-methyl-1-(6-methylpyridin-2-yl)-2-thia-1,8-diazaspiro[4.5]dec-3-ene-8-carboxylate 2,2-dioxide (C35) (1.7 mg, 0.0040 mmol) in methanol (50 μL). The resulting solution was stirred for 20 hours. Volatiles were removed in vacuo to give (5R,7S)-7-methyl-1-(6-methylpyridin-2-yl)-2-thia-1,8-diazaspiro[4.5]dec-3-ene 2,2-dioxide as a yellow solid. The solid was treated with acetonitrile (100 μL), 1-(bromomethyl)-3-isopropoxybenzene (0.90 mg, 0.0040 mmol) and potassium carbonate (5.0 mg, 0.036 mmol) and stirred for 20 hours. The reaction mixture was loaded onto an Oasis™ MCX SPE column, and the column was washed with dichloromethane (6 mL), then eluted with a solution of ammonia in methanol (1 M, 3 mL). The ammonia/methanol solution was concentrated in vacuo to give an amber residue, which was purified by silica gel chromatography (Eluant: 75% ethyl acetate in heptane). The product was obtained as a colorless oil. Yield: 0.50 mg, 0.00011 mmol, 30%. LCMS m/z 442.0 (M+1). ¹H NMR (500 MHz, CDCl₃) δ 7.59 (t, J=7.8 Hz, 1H), 7.45 (d, J=8.1 Hz, 1H), 7.17-7.22 (m, 2H), 7.00 (d, J=7.6 Hz, 1H), 6.87 (br s, 1H), 6.85 (d, J=7.6 Hz, 1H), 6.77 (dd, J=8.3, 2.0 Hz, 1H), 6.69 (d, J=7.1 Hz, 1H), 4.49-4.59 (m, 1H), 3.82 (d, J=13.6 Hz, 1H), 3.42 (d, J=13.5 Hz, 1H), 3.16 (dd, J=13.5, 5.5 Hz, 1H), 3.12-3.02 (m, 1H), 2.89 (dd, J=12.0, 5.9 Hz, 1H), 2.77 (ddd, J=12.9, 9.0, 4.0 Hz, 1H), 2.57 (s, 3H), 2.36-2.44 (m, 1H), 1.66-1.73 (m, 1H), 1.57-1.62 (m, 1H), 1.34 (d, J=6.1 Hz, 6H), 1.16 (d, J=6.6 Hz, 3H).

### Method A

### Preparation of 8-substituted (5R,7S)-2-thia-1,8-diazaspiro[4.5]dec-3-ene 2,2-dioxides and 8-substituted (5R,7S)-2-thia-1,8-diazaspiro[4.5]decane 2,2-dioxides via reductive amination

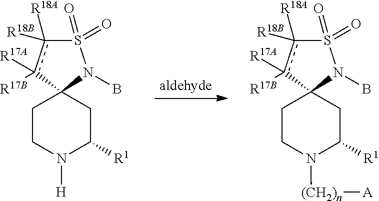

A solution of the appropriate (5R,7S)-2-thia-1,8-diazaspiro[4.5]dec-3-ene 2,2-dioxide or (5R,7S)-2-thia-1,8-diazaspiro[4.5]decane 2,2-dioxide in 1,2-dichloroethane (0.05-0.1 M) was treated with the requisite aldehyde (1.5-2 equivalents) and acetic acid (1 equivalent). In some cases, 4 Å molecular sieves were added. The reaction mixture was stirred for 3 to 18 hours, at a temperature of 25° C. to 50° C. After addition of sodium triacetoxyborohydride (2 equivalents), stirring was continued until the reaction was complete as assessed by thin layer chromatographic or mass spectral analysis. In some cases, heat was applied. Additional quantities of reagents were added if necessary. When the reaction was complete, it was partitioned between ethyl acetate and aqueous sodium bicarbonate solution. The aqueous layer was extracted with ethyl acetate, and the combined organic layers were dried over sodium sulfate, filtered and concentrated. Purification of the crude product was carried out using one of the following methods: 1) silica gel chromatography using an appropriate gradient: methanol in dichloromethane, ethyl acetate in heptane or 2-propanol in ethyl acetate; 2) reversed-phase HPLC (Column: Waters XBridge C₁₈, 5 μm; Mobile phase A: 0.03% NH₄OH in Water (v/v); Mobile phase B: 0.03% NH₄OH in Acetonitrile (v/v); Gradient: 15% to 100% B).

### Method B

### Preparation of 8-substituted (5R,7S)-2-thia-1,8-diazaspiro[4.5]dec-3-ene 2,2-dioxides and 8-substituted (5R,7S)-2-thia-1,8-diazaspiro[4.5]decane 2,2-dioxides via alkylation

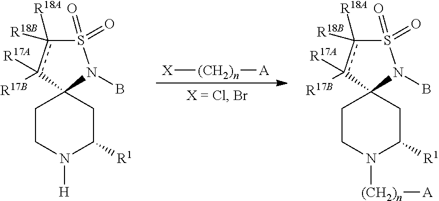

A solution of the appropriate (5R,7S)-2-thia-1,8-diazaspiro[4.5]dec-3-ene 2,2-dioxide or (5R,7S)-2-thia-1,8-diazaspiro[4.5]decane 2,2-dioxide in N,N-dimethylformamide (0.1-0.3 M) was treated with the requisite alkylating agent (1.2-2 equivalents). For chloro compounds, potassium carbonate (2-3 equivalents) was added, and the reaction mixture was heated at 80° C. for 5 hours. For bromo compounds, cesium carbonate (3 equivalents) was used as the base, and the reaction was carried out at room temperature for 18 hours to 5 days. In both cases, when the reaction was complete, it was partitioned between ethyl acetate and water. The aqueous layer was extracted with additional ethyl acetate, and the combined organic layers were washed with water, then with saturated aqueous sodium chloride solution, and dried over sodium sulfate. After filtration and removal of solvent, the residue was purified by one of the following methods: 1) Silica gel chromatography using an appropriate gradient of methanol in dichloromethane, ethyl acetate in heptane or 2-propanol in ethyl acetate; 2) Reversed-phase HPLC (Column: Phenomenex Phenyl-Hexyl, 5 μm; Mobile phase A: 0.1% formic acid in water, Mobile phase B: 0.1% formic acid in methanol; Gradient: 5% to 100% B).

In some cases where compounds were prepared by Methods A or B, the final compound was converted to its hydrochloride salt. This was effected either by: 1) dissolving the free base in diethyl ether and treating it with a solution of hydrogen chloride in diethyl ether (2 N, 1 equivalent), followed by isolation of the hydrochloride salt via filtration; or 2) treating a methanolic solution of the free base with a solution of hydrogen chloride in dioxane (4 M), followed by removal of solvent and appropriate trituration of the residue.

### Method C

### Preparation of (5R,7S)-2-thia-1,8-diazaspiro[4.5]decane 2,2-dioxides via hydrogenation

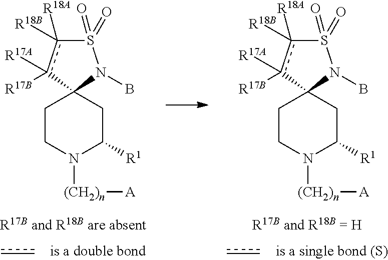

The substrate (0.04-0.15 mmol) in methanol (2-5 mL) was hydrogenated using an H-Cube® continuous flow reactor (ThalesNano) (20-30° C., 10% Pd/C, 1 atmosphere H2). The eluant was concentrated in vacuo; if purification was required, the material was purified by one of the following methods. 1) Preparative plate chromatography on silica gel (Eluant: 2-propanol in ethyl acetate); 2) Reversed-phase HPLC (Column: Phenomenex Gemini-NX, 5 μm; Mobile phase A: 0.1% NH₄OH in water, Mobile phase B: 0.1% NH₄OH in methanol; Gradient: 5% to 100% B); 3) Reversed-phase HPLC (Column: Waters XBridge C18, 5 μm; Mobile phase A: 0.03% NH₄OH in water, Mobile phase B: 0.03% NH₄OH in acetonitrile; Gradient: 15% to 100% B; 4) Reversed-phase HPLC (Column: Waters Sunfire C18, 5 μm; Mobile phase A: 0.05% formic acid in water, Mobile phase B: 0.05% formic acid in acetonitrile; Gradient: 20% to 100% B; 5) Chromatography on a Chiralcel OD column, 10 μm (Mobile phase: 80/20 CO₂/methanol).

The structures of additional Examples are shown in Tables 11, 12 and 13, which also give physical data and preparative information for these Examples. For starting materials that are not commercially available, preparation is described in a footnote. Biological activity for many of the Examples is given in Table 14.

TABLE 11
Examples 5-62 and 69-80

| | |
|---|---|
| 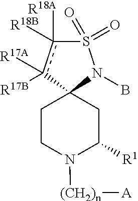 | B = 3-fluorophenyl R¹ = CH₃ R^{17A}, R^{18A} = H R^{17B}, R^{17B} (if present) = H |
| | 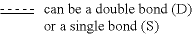 |

| Ex # | 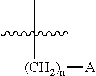 | 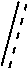 | Method of preparation; starting material(s) | IUPAC Name | ¹H NMR (400 MHz, CDCl₃), δ (ppm); Mass spectrum, observed ion m/z (M + 1) (unless otherwise indicated) or HPLC retention time (minutes); Mass spectrum m/z (M + 1) |
|---|---|---|---|---|---|
| 5 | 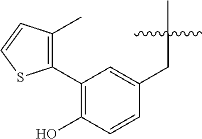 [SMILES: *Cc1ccc(O)c(-c2sccc2C)c1] | D | A; P1¹ | 4-{[(5R,7S)-1-(3- fluorophenyl)-7- methyl-2,2- dioxido-2-thia-1,8- diazaspiro[4.5]dec- 3-en-8-yl]methyl}- 2-(3-methyl-2- thienyl)phenol, hydrochloride salt | 7.42 (ddd, J = 8.2, 8.2, 6.5 Hz, 1H), 7.35 (d, J = 5.2 Hz, 1H), 7.10-7.22 (m, 4H), 7.07 (d, J = 7.2 Hz. 1H), 7.05 (d, J = 2.0 Hz, 1H), 6.99 (d, J = 5.2 Hz, 1H), 6.90 (d, J = 8.3 Hz, 1H), 6.80 (d, J = 7.2 Hz, 1H), 5.14 (br s, 1H), 3.61 (d, J = 13.6 Hz, 1H), 3.25 (d, J = 13.3 Hz, 1H), 2.76-2.84 (m, 1H), 2.63 (br ddd. J = 13, 8, 4 Hz, 1H), 2.27 (br ddd, J = 13, 7, 4 Hz, 1H), 2.11 (s, 3H), 2.10-2.16 (m, 1H), 1.99 (br ddd, J = 13, 8, 4 Hz, 1H), 1.82-1.89 (m, 1H), 1.76 (br dd, J = 14, 6 Hz, 1H), 1.12 (d, J = 6.5 Hz, 3H);² 499.1 |
| 6 | 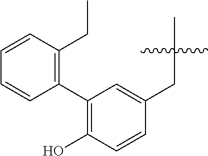 [SMILES: *Cc1ccc(O)c(-c2ccccc2CC)c1] | D | A; P1³ | 2′-ethyl-5- {[(5R,7S)-1-(3- fluorophenyl)-7- methyl-2,2- dioxido-2-thia-1,8- diazaspiro[4.5]dec- 3-en-8- yl]methyl}biphenyl- 2-ol | 7.36-7.45 (m, 3H), 7.28-7.31 (m, 1H), 7.05-7.22 (m, 6H), 6.97-6.99 (m, 1H), 6.89 (d, J = 8.3 Hz, 1H), 6.79 (d, J = 7.1 Hz, 1H), 4.68 (br s, 1H), 3.65 and 3.60 (2 br d, J = 13 Hz, 1H), 3.27 and 3.22 (2 br d, J = 13 Hz, 1H), 2.75-2.87 (m, 1H), 2.58-2.64 (m, 1H), 2.35-2.53 (m, 2H), 2.24-2.33 (m, 1H), 2.08-2.15 (m, 1H), 1.93-2.02 (m, 1H), 1.80-1.87 (m, 1H), 1.72-1.78 (m, 1H), 1.09-1.13 (m, 3H), 1.02 (t, J = 7.6 Hz, 3H); 507.2 |
| 7 | 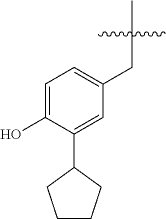 [SMILES: *Cc1ccc(O)c(C2CCCC2)c1] | D | A; P1⁴ | 2-cyclopentyl-4- {[(5R,7S)-1-(3- fluorophenyl)-7- methyl-2,2- dioxido-2-thia-1,8- diazaspiro[4.5]dec- 3-en-8- yl]methyl}phenol | 7.40 (td, J = 8.1, 6.6 Hz, 1H), 7.15-7.23 (m, 2H), 7.13 (dt, J = 9.3, 2.2 Hz, 1H), 7.07 (d, J = 7.2 Hz, 1H), 6.97 (d, J = 2.0 Hz, 1H), 6.87 (dd, J = 8.1, 2.0 Hz, 1H), 6.79 (d, J = 7.2 Hz, 1H), 6.65 (d, J = 8.2 Hz, 1H), 3.59 (d, J = 13.3 Hz, 1H), 3.27 (d, J = 13.1 Hz, 1H), 3.12-3.22 (m, 1H), 2.72-2.82 (m, 1H), 2.56-2.65 (m, 1H), 2.21-2.29 (m, 1H), 2.13 (dd, J = 13.8, 4.6 Hz, 1H), 1.94-2.07 (m, 2H). 1.63-1.91 (m, 4H), 1.50-1.63 (m, 5H), 1.13 (d, J = 6.2 Hz, 3H); 471.1 |
| 8 | 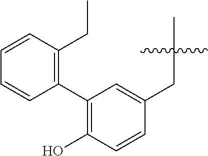 [SMILES: *Cc1ccc(O)c(-c2ccccc2CC)c1] | S | C; Example 6 | 2′-ethyl-5- {[(5R,7S)-1-(3- fluorophenyl)-7- methyl-2,2- dioxido-2-thia-1,8- diazaspiro[4.5]dec- 8- yl]methyl}biphenyl- 2-ol | 7.25-7.42 (m, 4H), 7.05-7.19 (m, 4H), 7.02 (br d, J = 9.4 Hz, 1H), 6.87-6.99 (m, 2H), 3.68-3.85 (m. 1H), 3.45-3.61 (m, 1H), 3.32-3.42 (m, 2H), 2.67-2.89 (m, 3H), 2.33-2.55 (m, 5H), 1.92-2.20 (m, 3H), 1.27 (m, 3H), 1.03 (t, J = 7.5 Hz, 3H); 509.1 |
| 9 | 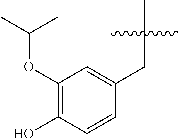 [SMILES: *Cc1ccc(O)c(OC(C)C)c1] | D | A; P1, P8 | 4-{[(5R,7S)-1-(3- ftuorophenyl)-7- methyl-2,2- dioxido-2-thia-1,8- diazaspiro[4.5]dec- 3-en-8-yl]methyl}- 2- isopropoxyphenol | 7.41 (dt, J = 8.2, 6.4 Hz, 1H), 7.17-7.23 (m, 2H), 7.14 (dt, J = 9.3, 2.2 Hz, 1H), 7.08 (d, J = 7.2 Hz, 1H), 6.81 (d, J = 8.1 Hz, 1H), 6.80 (d, J = 7.2 Hz, 1H), 6.71 (d, J = 1.8 Hz, 1H), 6.64 (dd, J = 8.1, 1.9 Hz, 1H), 5.61 (s, 1H), 4.51 (septet, J = 6.1 Hz, 1H), 3.56 (d, J = 13.3 Hz, 1H), 3.25 (d, J = 13.3 Hz, 1H), 2.76-2.84 (m, 1H), 2.60 (ddd, J = 12.6, 8.6, 3.7 Hz, 1H), 2.26 (ddd, J = 12.4, 7.3, 3.9 Hz, 1H), 2.13 (dd, J = 13.6, 5.0 Hz, 1H), 1.98 (ddd, J = 12.9, 8.7, 3.8 Hz, 1H), 1.79- 1.87 (m, 1H), 1.75 (dd, J = 13.8, 5.0 Hz, 1H), 1.33 (d, J = 6.0 Hz, 3H), 1.32 (d, J = 6.0 Hz, 3H), 1.11 (d, J = 6.6 Hz, 3H); 461.0 |
| 10 | 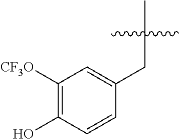 | D | A; P1 | 4-{[(5R,7S)-1-(3- fluorophenyl)-7- methyl-2,2- dioxido-2-thia-1,8- diazaspiro[4.5]dec- 3-en-8-yl]methyl}- 2- (trifluoromethoxy) phenol, hydrochloride salt | ¹H NMR (400 MHz, DMSO-d₆) δ 6.96- 7.75 (m, 9H), 4.08-4.21 (m, 2H), 3.23- 3.31 (m, 1H), 2.99-3.07 (m, 1H), 2.53- 2.64 (m, 1H), 2.08-2.41 (m. 4H), 1.95- 2.04 (m, 1H), 1.41-1.45 (m, 3H); 486.9 |
| 11 | 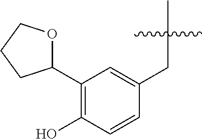 [SMILES: *Cc1ccc(O)c(C2CCCO2)c1] | D | A; P1⁵ | 4-{[(5R,7S)-1-(3- fluorophenyl)-7- methyl-2,2- dioxido-2-thia-1,8- diazaspiro[4.5]dec- 3-en-8-yl]methyl}- 2-(tetrahydrofuran- 2-yl)phenol (mixture of diastereomers at the tetrahydrofuran substituent) | 8.43 (br s, 1H), 7.36-7.45 (m, 1H), 7.17- 7.24 (m, 2H), 7.13 (br d, J = 9.7 Hz, 1H), 7.06 and 7.05 (2 d, J = 7.2, 1H), 6.96 (dt, J = 8.2, 2.5 Hz, 1H), 6.73-6.81 (m, 3H), 4.94 (ddd, J = 9.1, 6.2, 2.9 Hz, 1H), 4.10-4.16 (m, 1H), 3.96 (td, J = 8.2, 6.0 Hz, 1H), 3.50-3.60 (m, 1H), 3.18-3.26 (m, 1H), 2.70-2.81 (m, 1H), 2.52-2.64 (m, 1H), 2.18-2.36 (m, 2H), 1.71-2.16 (m, 7H), 1.12 and 1.11 (2 d, J = 6.5 Hz, 3H); 473.6 |
| 12 | 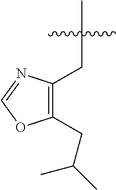 [SMILES: *Cc1ncoc1CC(C)C] | D | A; P1⁶ | (5R,7S)-1-(3- fluorophenyl)-8- [(5-isobutyl-1,3- oxazol-4- yl)methyl]-7- methyl-2-thia-1,8- diazaspiro[4.5]dec- 3-ene 2,2-dioxide | ¹H NMR (500 MHz, CDCl₃) δ 7.66 (s, 1H), 7.39 (dt, J = 8.1, 6.5 Hz, 1H), 7.14- 7.21 (m, 2H), 7.12 (dt, J = 9.2, 2.2 Hz, 1H), 7.01 (d, J = 7.1 Hz, 1H), 6.79 (d, J = 7.3 Hz, 1H), 3.49 (d, J = 13.9 Hz, 1H), 3.35 (br d, J = 13.7 Hz, 1H), 2.78-2.84 (m, 1H), 2.58-2.65 (m, 1H), 2.41 (d, J = 7.1 Hz, 2H), 2.32 (ddd, J = 12.1, 8.1, 3.9 Hz, 1H), 2.15 (dd, J = 13.4, 3.9 Hz, 1H), 2.00-2.08 (m, 1H), 1.87-1.97 (m, 2H), 1.74-1.81 (m, 1H), 1.16 (d, J = 6.3 Hz, 3H), 0.87 (d, J = 6.7 Hz, 3H), 0.86 (d, J = 6.8 Hz, 3H); 434.1 |
| 13 | 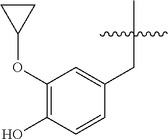 [SMILES: *Cc1ccc(O)c(OC2CC2)c1] | D | A; P1⁷ | 2-(cyclopropyloxy)- 4-{[(5R,7S)-1-(3- fluorophenyl)-7- methyl-2,2- dioxido-2-thia-1,8- diazaspiro[4.5]dec- 3-en-8- yl]methyl}phenol, hydrochloride salt | 7.25-7.31 (m, 1H), 7.09-7.20 (m, 3H), 7.05 (ddd, J = 9, 2, 2 Hz, 1H), 6.98 (d, J = 7.0 Hz, 1H), 6.87 (d, J = 7.0 Hz, 1H), 6.83 (d, J = 8.1 Hz, 1H), 6.55 (dd, J = 8.1, 2.0 Hz, 1H), 4.21 (d, J = 13.8 Hz, 1H), 3.78-3.85 (m, 2H), 2.78-3.11 (m, 4H), 2.14-2.36 (m, 3H), 1.63 (d, J = 5.8 Hz, 3H), 0.74-0.91 (m, 4H); 459.0 |
| 14 | 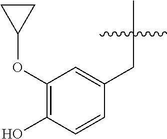 [SMILES: *Cc1ccc(O)c(OC2CC2)c1] | D | A; P1⁷ | 2-(cyclopropyloxy)- 4-{[(5R,7S)-1-(3- fluorophenyl)-7- methyl-2,2- dioxido-2-thia-1,8- diazaspiro[4.5]dec- 3-en-8- yl]methyl}phenol | 7.41 (td, J = 8.1, 6.4 Hz, 1H), 7.20 (dd, J = 8.3, 2.0 Hz, 1H), 7.11-7.16 (m, 2H), 7.09 (d, J = 7.2 Hz, 1H), 7.00 (d, J = 2.0 Hz, 1H), 6.80 (d, J = 7.2 Hz, 1H), 6.79 (d, J = 8.0 Hz, 1H), 6.67 (dd, J = 8.1, 1.8 Hz, 1H), 5.36 (br s, 1H), 3.71-3.76 (m, 1H), 3.58 (d, J = 13.5 Hz, 1H), 3.31 (d, J = 13.3 Hz, 1H), 2.77-2.87 (m, 1H), 2.57-2.66 (m, 1H), 2.29 (ddd, J = 12.6, 7.1, 3.7 Hz, 1H), 2.10-2.17 (m, 1H), 1.95-2.03 (m, 1H), 1.80-1.88 (m, 1H), 1.76 (ddd, J = 13.5, 5.9, 1.3 Hz, 1H), 1.13 (d, J = 6.6 Hz. 3H), 0.71-0.78 (m, 4H); 459.0 |
| 15 | 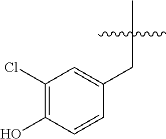 [SMILES: *Cc1ccc(O)c(Cl)c1] | D | A; P1 | 2-chloro-4- {[(5R,7S)-1-(3- fluorophenyl}-7- methyl-2,2- dioxido-2-thia-1,8- diazaspiro[4.5]dec- 3-en-8- yl]methyl}phenol, hydrochloride salt | 7.39-7.46 (m, 1H), 7.12-7.24 (m, 4H), 7.05 (d, J = 7.2 Hz, 1H), 6.97-7.01 (m, 1H), 6.91 (d, J = 8.4 Hz, 1H), 6.80 (d, J = 7.2 Hz, 1H), 5.49 (br s, 1H), 3.56 (d, J = 13.5 Hz, 1H), 3.23 (d, J = 13.5 Hz, 1H), 2.70-2.80 (m, 1H), 2.58 (ddd, J = 12.3, 8.2, 3.7 Hz, 1H), 2.22 (ddd, J = 12.6. 7.5, 4.0 Hz, 1H), 2.13 (dd, J = 13.9, 4.5 Hz, 1H), 1.94-2.02 (m, 1H), 1.82-1.89 (m, 1H), 1.76 (ddd, J = 13.9, 6.4, 1.2 Hz, 1H), 1.11 (d, J = 6.6 Hz, 3H);² 437.0 |
| 16 | 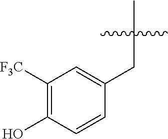 [SMILES: *Cc1ccc(O)c(C(F)(F)F)c1] | D | A; P1 | 4-{[(5R,7S)-1-(3- fluorophenyl)-7- methyl-2,2- dioxido-2-thia-1,8- diazaspiro[4.5]dec- 3-en-8-yl]methyl}- 2- (trifluoromethyl) phenol, hydrochloride salt | 7.42 (td, J = 8.2, 6.4 Hz, 1H), 7.31 (d, J = 1.8 Hz, 1H), 7.17-7.26 (m, 3H), 7.14 (dt, J = 9.3, 2.3 Hz, 1H), 7.05 (d, J = 7.2 Hz, 1H), 6.85 (d, J = 8.4 Hz, 1H), 6.81 (d, J = 7.2 Hz, 1H), 3.61 (d, J = 13.6 Hz, 1H), 3.27 (d, J = 13.6 Hz, 1H), 2.69-2.80 (m, 1H), 2.58 (ddd, J = 12.4, 8.1, 3.7 Hz, 1H), 2.17-2.25 (m, 1H), 2.14 (dd, J = 13.9, 4.5 Hz. 1H), 1.94-2.03 (m. 1H), 1.81-1.90 (m, 1H), 1.77 (ddd, J = 13.8, 6.4, 0.9 Hz, 1H), 1.12 (d, J = 6.4 Hz, 3H);² 470.9 |
| 17 | 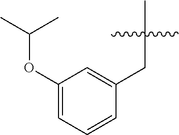 [SMILES: *Cc1cccc(OC(C)C)c1] | D | B; P1⁸ | (5R,7S)-1-(3- fluorophenyl)-8-(3- isopropoxybenzyl)- 7-methyl-2-thia- 1,8- diazaspiro[4.5]dec- 3-ene 2,2-dioxide | ¹H NMR (500 MHz, CDCl₃) δ 7.40-7.45 (m, 1H), 7.13-7.23 (m, 4H), 7.09 (d, J = 7.2 Hz, 1H), 6.80 (d, J = 7.2 Hz, 1H), 6.73-6.76 (m, 3H), 4.50 (septet, J = 6.0 Hz, 1H), 3.63 (d, J = 13.5 Hz, 1H), 3.25 (d, J = 13.4 Hz, 1H), 2.78-2.85 (m, 1H), 2.59-2.65 (m, 1H), 2.24-2.30 (m, 1H), 2.14 (dd, J = 13.7, 4.6 Hz, 1H), 1.95- 2.01 (m, 1H), 1.81-1.87 (m, 1H), 1.76 (br dd, J = 13.7, 6.0 Hz, 1H), 1.30-1.32 (m, 6H), 1.11 (d, J = 6.6 Hz, 3H); 445.1 |
| 18 | 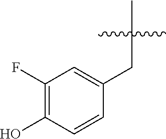 [SMILES: *Cc1ccc(O)c(F)c1] | D | A; P1 | 2-fluoro-4- {[(5R,7S)-1-(3- fluorophenyl)-7- methyl-2,2- dioxido-2-thia-1,8- diazaspiro[4.5]dec- 3-en-8- yl]methyl}phenol, hydrochloride salt | 7.42 (td, J = 8.1, 6.4 Hz, 1H), 7.17-7.24 (m, 2H), 7.14 (dt, J = 9.2, 2.2 Hz, 1H), 7.06 (d, J = 7.2 Hz, 1H), 6.94 (dd, J = 11.5, 1.8 Hz, 1H), 6.79-6.91 (m, 3H), 3.57 (d, J = 13.7 Hz, 1H), 3.24 (d, J = 13.7 Hz, 1H), 2.72-2.82 (m, 1H), 2.58 (ddd, J = 12.4, 8.3, 3.6 Hz, 1H), 2.20-2.28 (m, 1H), 2.13 (dd, J = 13.8, 4.6 Hz, 1H), 1.93-2.02 (m, 1H), 1.81-1.89 (m, 1H), 1.76 (dd, J = 13.8, 6.34 Hz, 1H), 1.11 (d, J = 6.6 Hz, 3H);² 421.0 |
| 19 | 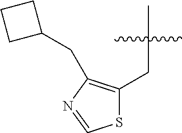 [SMILES: *Cc1scnc1CC1CCC1] | D | A; P1⁹ | (5R,7S)-8-{[4- (cyclobutylmethyl)- 1,3-thiazol-5- yl]methyl}-1-(3- fluorophenyl)-7- methyl-2-thia-1,8- diazaspiro[4.5]dec- 3-ene 2,2-dioxide | 8.59 (s, 1H), 7.43 (dt, J = 8.2, 6.4 Hz, 1H), 7.18-7.24 (m, 2H), 7.12-7.17 (m, 1H), 7.07 (d, J = 7.2 Hz, 1H), 6.81 (d, J = 7.2 Hz, 1H), 3.79 (d, J = 14.4 Hz, 1H), 3.48 (d, J = 14.2 Hz, 1H), 2.81-2.92 (m, 1H), 2.56-2.77 (m, 4H), 2.25-2.34 (m, 1H), 2.13 (dd, J = 13.7, 4.7 Hz, 1H), 1.91-2.02 (m, 3H), 1.58-1.89 (m, 6H), 1.13 (d, J = 6.6 Hz, 3H); 462.1 |
| 20 | 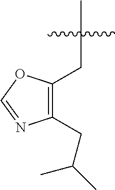 [SMILES: *Cc1ocnc1CC(C)C] | D | A; P1¹⁰ | (5R,7S)-1-(3- fluorophenyl)-8- [(4-isobutyl-1,3- oxazol-5- yl)methyl]-7- methyl-2-thia-1,8- diazaspiro[4.5]dec- 3-ene 2,2-dioxide | ¹H NMR (500 MHz, CDCl₃) δ 7.68 (s, 1H), 7.34-7.40 (m, 1H), 7.19 (dd, J = 8.0, 2.2 Hz, 1H), 7.15-7.20 (m, 1H), 7.13 (dt, J = 9.3, 2.2 Hz, 1H), 6.92 (d, J = 7.2 Hz, 1H), 6.80 (d, J = 7.2 Hz, 1H), 3.57 (AB quartet, J_{AB} =14.9 Hz, Δν_{AB} = 34.4 Hz, 2H), 2.54-2.66 (m, 2H), 2.23 (d, J = 7.1 Hz, 2H), 2.20-2.27 (m, 1H), 2.12 (ddd, J = 14.1, 4.0, 1.5 Hz, 1H), 2.02- 2.08 (m, 1H), 1.90-2.01 (m, 2H), 1.76 (dd, J = 14.0, 7.9 Hz, 1H), 1.15 (d, J = 6.6 Hz, 3H), 0.85-0.88 (m, 6H); 434.1 |
| 21 | 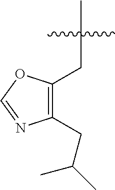 [SMILES: *Cc1ocnc1CC(C)C] | S | C; Example 20 | (5R,7S)-1-(3- fluorophenyl)-8- [(4-isobutyl-1,3- oxazol-5- yl)methyl]-7- methyl-2-thia-1,8- diazaspiro[4.5]dec ane 2,2-dioxide | 7.69 (s, 1H), 7.33 (td, J = 8.1, 6.4 Hz, 1H), 7.09-7.16 (m, 2H), 7.06 (dt, J = 9.3, 2.2 Hz, 1H), 3.54 (AB quartet, J_{AB} =14.6 Hz, Δν_{AB} = 22.6 Hz, 2H), 3.33-3.39 (m, 2H), 2.58-2.67 (m, 1H), 2.51-2.58 (m, 1H), 2.37-2.50 (m, 2H), 2.23-2.30 (m, 1H), 2.22 (d, J = 7.0 Hz, 2H), 2.03-2.15 (m, 2H), 1.90-2.01 (m, 1H), 1.74-1.84 (m, 1H), 1.63 (dd, J = 13.8, 7.3 Hz, 1H), 1.12 (d, J = 6.4 Hz. 3H), 0.85 (d, J = 6.6 Hz, 3H), 0.86 (d, J = 6.4 Hz, 3H); 436.2 |
| 22 | 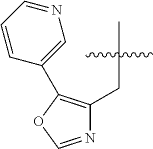 [SMILES: *Cc1ncoc1-c1cccnc1] | D | A; P1¹¹ | (5R,7S)-1-(3- fluorophenyl)-7- methyl-8-[(5- pyridin-3-yl-1,3- oxazol-4- yl)methyl]-2-thia- 1,8- diazaspiro[4.5]dec- 3-ene 2,2-dioxide, hydrochloride salt | 8.89 (dd, J = 2.3, 0.8 Hz, 1H), 8.53 (dd, J = 4.8, 1.7 Hz, 1H), 7.93 (dt, J = 8.0, 2.0 Hz, 1H), 7.85 (s, 1H), 7.37-7.44 (m, 1H), 7.26-7.30 (m, 1H), 7.15-7.22 (m, 2H), 7.10 (dt, J = 9.4, 2.2 Hz, 1H), 7.04 (d, J = 7.2 Hz, 1H), 6.79 (d, J = 7.2 Hz, 1H), 3.79 (d, J = 13.7 Hz, 1H), 3.50 (d, J = 13.9 Hz, 1H), 2.84-2.93 (m, 1H), 2.68 (ddd, J = 12.5, 8.5, 3.5 Hz, 1H), 2.36- 2.44 (m, 1H), 2.10 (dd, J = 13.0, 4.7 Hz, 1H), 1.89-1.97 (m, 1H), 1.80-1.85 (m, 1H), 1.73-1.80 (m, 2H), 1.15 (d, J = 6.6 Hz, 3H);² 455.7 |
| 23 | 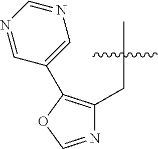 [SMILES: *Cc1ncoc1-c1cncnc1] | D | A; P1¹² | (5R,7S)-1-(3- fluorophenyl)-7- methyl-8-[(5- pyrimidin-5-yl-1,3- oxazol-4- yl)methyl]-2-thia- 1,8- diazaspiro[4.5]dec- 3-ene 2,2-dioxide, hydrochloride salt | 9.09 (s, 1H), 9.01 (s, 2H), 7.91 (s, 1H), 7.42 (td, J = 8.2, 6.3 Hz, 1H), 7.15-7.23 (m, 2H), 7.09 (dt, J = 9.2, 2.2 Hz, 1H), 7.04 (d, J = 7.2 Hz. 1H), 6.80 (d, J = 7.2 Hz, 1H), 3.81 (d, J = 13.9 Hz, 1H), 3.49 (d, J = 13.9 Hz, 1H), 2.84-2.93 (m, 1H), 2.68 (ddd, J = 12.5, 8.6, 3.6 Hz, 1H), 2.37 (ddd, J = 12.5, 6.9, 3.6 Hz, 1H), 2.09 (dd, J = 13.6, 4.8 Hz, 1H), 1.86- 1.94 (m, 1H), 1.74-1.83 (m, 2H), 1.17 (d, J = 6.6 Hz, 3H);² 456.7 |
| 24 | 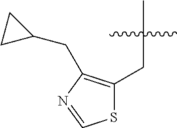 [SMILES: *Cc1scnc1CC1CC1] | D | A; P1¹³ | (5R,7S)-8-{[4- (cyclopropylmethyl)- 1,3-thiazol-5- yl]methyl}-1-(3- fluorophenyl)-7- methyl-2-thia-1,8- diazaspiro[4.5]dec- 3-ene 2,2-dioxide, hydrochloride salt | 8.63 (s, 1H), 7.44 (td, J = 8.2, 6.5 Hz, 1H), 7.19-7.25 (m, 2H), 7.15 (dt, J = 9.3, 2.2 Hz, 1H), 7.06 (d, J = 7.2 Hz, 1H), 6.82 (d, J = 7.2 Hz, 1H), 3.79 (d, J = 14.4 Hz, 1H), 3.47 (d, J = 14.3 Hz, 1H), 2.82- 2.91 (m, 1H), 2.64 (ddd, J = 12.3, 8.2, 3.8 Hz, 1H), 2.57 (d, J = 6.8 Hz, 2H), 2.26-2.34 (m, 1H), 2.14 (dd, J = 13.8, 4.4 Hz, 1H), 1.99 (ddd, J = 13.2, 8.8, 4.0 Hz, 1H), 1.82-1.90 (m, 1H), 1.76 (ddd, J = 13.7, 6.2, 1.1 Hz, 1H), 1.13 (d, J = 6.6 Hz, 3H), 0.96-1.05 (m, 1H), 0.43-0.49 (m, 2H), 0.13-0.17 (m, 2H);² 448.7 |
| 25 | 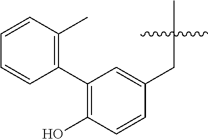 [SMILES: *Cc1ccc(O)c(-c2ccccc2C)c1] | D | A; P1³ | 5-{[(5R,7S)-1-(3- fluorophenyl)-7- methyl-2,2- dioxido-2-thia-1,8- diazaspiro[4.5]dec- 3-en-8-yl]methyl}- 2′-methylbiphenyl- 2-ol, hydrochloride salt | 7.42 (td, J = 8.1, 6.2 Hz, 1H), 7.28-7.35 (m, 3H), 7.21 (dd, J = 8.3, 1.8 Hz, 1H), 7.05-7.22 (m, 5H), 6.95 (d, J = 2.2 Hz, 1H), 6.89 (d, J = 8.2 Hz, 1H), 6.80 (d, J = 7.2 Hz, 1H), 4.71 (br s, 1H), 3.63 (d, J = 13.3 Hz, 1H), 3.24 (d, J = 13.3 Hz, 1H), 2.75-2.85 (m, 1H), 2.63 (ddd, J = 12.4, 8.3, 3.5 Hz, 1H), 2.23-2.32 (m, 1H), 2.14 (s, 3H), 2.10-2.15 (m, 1H), 1.94-2.03 (m, 1H), 1.80-1.88 (m, 1H), 1.75 (ddd, J = 13.7, 6.2, 1.3 Hz, 1H), 1.11 (d, J = 6.6 Hz, 3H);² 493.7 |
| 26 | 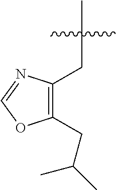 [SMILES: *Cc1ncoc1CC(C)C] | S | C; Example 12 | (5R,7S)-1-(3- fluorophenyl)-8- [(5-isobutyl-1,3- oxazol-4- yl)methyl]-7- methyl-2-thia-1,8- diazaspiro[4.5]dec ane 2,2-dioxide, formic acid salt | ¹H NMR (500 MHz, CDCl₃) δ 8.04 (s, 1H), 7.74 (s, 1H), 7.29-7.36 (m, 1H), 7.12 (td, J = 8.1, 2.1 Hz, 1H), 7.07 (d, J = 7.8 Hz, 1H), 7.01 (dt, J = 9.1, 2.2 Hz, 1H), 4.23 (d, J = 15.6 Hz, 1H), 3.94 (d, J = 15.1 Hz, 1H), 3.44 (t, J = 7.3 Hz, 2H), 3.12-3.21 (m, 2H), 2.32-2.53 (m, 9H), 1.95 (m, 1H), 1.52 (d, J = 8.3 Hz, 3H), 0.88-0.93 (m, 3H), 0.86 (d, J = 6.6 Hz, 3H); 436.1 |
| 27 | 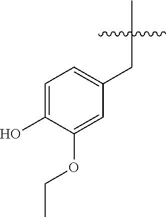 [SMILES: *Cc1ccc(O)c(OCC)c1] | D | A; P1 | 2-ethoxy-4- {[(5R,7S)-1-(3- fluorophenyl)-7- methyl-2,2- dioxido-2-thia-1,8- diazaspiro[4.5]dec- 3-en-8- yl]methyl}phenol | 7.40 (td, J = 8.1, 6.5 Hz, 1H), 7.17-7.23 (m, 2H), 7.14 (dt, J = 9.3, 2.2 Hz, 1H), 7.07 (d, J = 7.2 Hz, 1H), 6.80 (dd, J = 7.6, 3.1 Hz, 2H), 6.68 (s, 1H), 6.64 (dd, J = 8.1, 1.7 Hz, 1H), 5.59 (brs, 1H), 4.05 (q, J = 7.0 Hz, 2H), 3.57 (d, J = 12.7 Hz, 1H), 3.25 (d, J = 12.5 Hz, 1H), 2.73-2.83 (m, 1H), 2.55-2.65 (m, 1H), 2.25 (ddd, J = 12.3, 7.8, 4.1 Hz, 1H), 2.13 (dd, J = 13.5, 4.5 Hz, 1H), 1.94-2.03 (m, 1H), 1.72-1.90 (m, 2H), 1.43 (t, J = 6.9 Hz, 3H), 1.12 (d, J = 6.1 Hz, 3H); 447.1 |
| 28 | 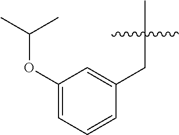 [SMILES: *Cc1cccc(OC(C)C)c1] | S | B; P4⁸ | (5R,7S)-1-(3- fluorophenyl)-8-(3- isopropoxybenzyl)- 7-methyl-2-thia- 1,8- diazaspiro[4.5]dec ane 2,2-dioxide | 7.38 (td, J = 8.1, 6.5 Hz, 1H), 7.11-7.19 (m, 3H), 7.08 (dt, J = 9.4, 2.2 Hz, 1H), 6.71-6.78 (m, 3H), 4.50 (septet, J = 6.0 Hz, 1H), 3.56 (d, J = 13.7 Hz, 1H), 3.28- 3.39 (m, 3H), 2.78-2.88 (m, 1H), 2.43- 2.62 (m, 3H), 2.32 (ddd, J = 12.5, 6.0, 4.1 Hz, 1H), 2.03 (dd, J = 13.5, 5.1 Hz, 1H), 1.95 (ddd, J = 13.2, 9.2, 4.1 Hz, 1H), 1.70-1.78 (m, 1H), 1.66 (ddd, J = 13.5, 5.2, 1.7 Hz, 1H), 1.31 (d, J = 6.0 Hz, 6H), 1.09 (d, J = 6.8 Hz. 3H); 447.6 |
| 29 | 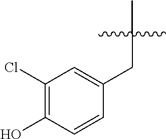 [SMILES: *Cc1ccc(O)c(Cl)c1] | S | A; P4 | 2-chloro-4- {[(5R7S)-1-(3- fluorophenyl)-7- methyl-2,2- dioxido-2-thia-1,8- diazaspiro[4.5]dec- 8-yl]methyl}phenol, hydrochloride salt | 12.28 (br s, 1H), 7.09 (td, J = 8.3, 6.4 Hz, 1H), 6.96 (td, J = 8.0, 2.2 Hz, 1H), 6.79-6.88 (m, 5H), 3.81-3.96 (m, 2H), 3.27-3.33 (m, 2H), 2.94 (br d, J = 13 Hz, 1H), 2.21-2.63 (m, 8H), 1.45 (d, J = 6.2 Hz, 3H); 439.6 |
| 30 | 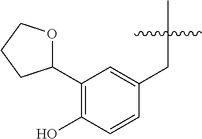 [SMILES: *Cc1ccc(O)c(C2CCCO2)c1] | D | Separation of diastereomers in Example 11; earlier- eluting isomer¹⁶ | 4-{[(5R,7S)-1-(3- fluorophenyl)-7- methyl-2,2- dioxido-2-thia-1,8- diazaspiro[4.5]dec- 3-en-8-yl]methyl}- 2-(tetrahydrofuran- 2-yl)phenol (single isomer at tetrahydrofuran- absolute stereochemistry not assigned) | ¹H NMR (500 MHz, CDCl₃) δ 8.41 (br s, 1H), 7.38-7.44 (m, 1H), 7.18-7.23 (m, 2H), 7.13 (dt, J = 9.3, 2.2 Hz, 1H), 7.05 (d, J = 7.3 Hz, 1H), 6.95 (dd, J = 8.2, 2.1 Hz, 1H), 6.74-6.80 (m, 3H), 4.94 (dd, J = 9.2, 6.2 Hz, 1H), 4.09-4.15 (m, 1H), 3.96 (td. J = 8.3, 6.1 Hz, 1H), 3.56 (d. J = 13.2 Hz, 1H), 3.20 (d, J = 13.2 Hz, 1H), 2.70-2.77 (m, 1H), 2.58 (ddd, J = 12.4, 8.3, 3.7 Hz, 1H), 2.27-2.35 (m, 1H), 2.23 (ddd, J = 12.2, 7.8, 3.9 Hz, 1H), 1.90-2.14 (m, 5H), 1.80-1.87 (m, 1H), 1.75 (dd, J = 13.4, 6.4 Hz, 1H), 1.10 (d, J = 6.6 Hz, 3H); 473.1 |
| 31 | 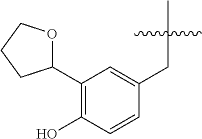 [SMILES: *Cc1ccc(O)c(C2CCCO2)c1] | D | Separation of diastereomers in Example 11; later- eluting isomer¹⁶ | 4-{[(5R,7S)-1-(3- fluorophenyl)-7- methyl-2,2- dioxido-2-thia-1,8- diazaspiro[4.5]dec- 3-en-8-yl]methyl}- 2-(tetrahydrofuran- 2-yl)phenol (single isomer at tetrahydrofuran- absolute stereochemistry not assigned) | ¹H NMR (500 MHz, CDCl₃) δ 8.41 (br s, 1H), 7.38-7.43 (m, 1H), 7.17-7.23 (m, 2H), 7.13 (dt, J = 9.3, 2.2 Hz, 1H), 7.06 (d, J = 7.1 Hz, 1H), 6.96 (dd, J = 8.3, 2.0 Hz, 1H), 6.74-6.80 (m, 3H), 4.94 (dd, J = 9.3, 6.1 Hz, 1H), 4.09-4.16 (m, 1H), 3.95 (td, J = 8.3, 6.1 Hz, 1H), 3.54 (d, J = 13.2 Hz, 1H), 3.23 (d, J = 13.2 Hz, 1H), 2.74-2.81 (m, 1H), 2.57 (ddd, J = 12.4, 8.4, 3.7 Hz, 1H), 2.26-2.34 (m, 1H), 2.19-2.26 (m, 1H), 1.90-2.15 (m, 5H), 1.79-1.86 (m, 1H), 1.75 (dd, J = 13.8, 6.2 Hz, 1H), 1.11 (d, J = 6.4 Hz, 3H); 473.1 |
| 32 | 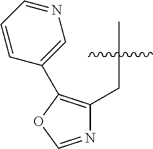 [SMILES: *Cc1ncoc1-c1cccnc1] | S | A; P4¹¹ | (5R,7S)-1-(3- fluorophenyl)-7- methyl-8-[(5- pyridin-3-yl-1,3- oxazol-4- yl)methyl]-2-thia- 1,8- diazaspiro[4.5]dec ane 2,2-dioxide, hydrochloride salt | 12.94 (br s, 1H), 8.89 (br s, 1H), 8.70 (dd, J = 4.7, 1.0 Hz, 1H), 7.94 (s, 1H), 7.91 (br d, J = 8 Hz, 1H), 7.53 (dd, J = 7.2, 5.5 Hz, 1H), 7.40 (td, J = 8.2, 6.5 Hz, 1H), 7.21 (td, J = 8.2, 2.4 Hz, 1H), 7.07-7.13 (m, 2H), 4.62 (br d, J = 16 Hz, 1H), 4.26 (br d, J = 16 Hz, 1H), 3.41- 3.48 (m, 2H), 3.33 (br s, 1H), 3.06-3.11 (m, 1H), 2.72-2.83 (m, 2H). 2.40-2.59 (m, 4H), 2.32 (br d, J = 11 Hz, 1H), 1.47 (d, J = 6.2 Hz, 3H); 457.6 |
| 33 | 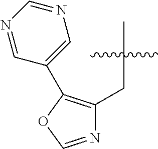 [SMILES: *Cc1ncoc1-c1cncnc1] | S | A; P4¹² | (5R,7S)-1-(3- fluorophenyl)-7- methyl-8-[(5- pyrimidin-5-yl-1,3- oxazol-4- yl)methyl]-2-thia- 1,8- diazaspiro[4.5]dec ane 2,2-dioxide, hydrochloride salt | 13.18 (br s, 1H), 9.31 (s, 1H), 8.94 (s, 2H), 8.01 (s, 1H), 7.40 (td, J = 8.0, 6.5 Hz, 1H), 7.18-7.24 (m, 1H), 7.07-7.15 (m, 2H), 4.44-4.50 (m, 1H), 4.17 (br d, J = 15 Hz, 1H), 3.44-3.49 (m, 2H), 3.30 (br s, 1H), 3.05-3.10 (m, 1H), 2.76-2.85 (m, 2H), 2.42-2.61 (m, 4H), 2.35 (br d, J = 11 Hz, 1H), 1.48 (d, J = 6.3 Hz, 3H); 458.6 |
| 34 | 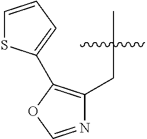 [SMILES: *Cc1ncoc1-c1cccs1] | D | B; P1¹⁴ | (5R,7S)-1-(3- fluorophenyl)-7- methyl-8-{[5-(2- thienyl)-1,3- oxazol-4- yl]methyl}-2-thia- 1,8- diazaspiro[4.5]dec- 3-ene 2,2-dioxide, hydrochloride salt | 13.03 (br s, 1H), 7.76 (s, 1H), 7.52 (dd, J = 5.2, 1.1 Hz, 1H), 7.37 (td, J = 8.2, 6.4 Hz, 1H), 7.32 (dd, J = 3.7, 1.2 Hz, 1H), 7.13-7.24 (m, 3H), 7.11 (dt, J = 9.0, 2.2 Hz, 1H), 6.88 (AB quartet, J_{AB} =7.0 Hz, Δν_{AB} = 5.4 Hz, 2H), 4.46-4.51 (m, 1H), 4.12 (br d, J = 15 Hz, 1H), 3.14-3.23 (m, 1H), 3.01-3.14 (m, 2H), 2.67-2.83 (m, 2H), 2.12-2.25 (m, 2H), 1.53 (d, J = 6.2 Hz, 3H); 460.6 |
| 35 | 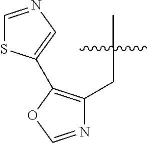 [SMILES: *Cc1ncoc1-c1cncs1] | D | B; P1¹⁴ | (5R,7S)-1-(3- fluorophenyl)-7- methyl-8-{[5-(1,3- thiazol-5-yl}-1,3- oxazol-4- yl]methyl}-2-thia- 1,8- diazaspiro[4.5]dec- 3-ene 2,2-dioxide | 8.79 (s, 1H), 8.12 (br s, 1H), 7.82 (s, 1H), 7.42 (td, J = 8.2, 6.3 Hz, 1H), 7.17- 7.24 (m, 2H), 7.13 (dt, J = 9.3, 2.2 Hz, 1H), 7.06 (br d, J = 7.0 Hz, 1H), 6.82 (d, J = 7.1 Hz, 1H), 3.78 (br d, J = 13 Hz, 1H), 3.54 (br s, 1H), 2.93 (br s, 1H), 2.70 (br s, 1H), 2.42 (br s, 1H), 2.18 (dd, J = 13.9, 4.7 Hz, 1H), 1.98-2.06 (m, 1H), 1.75-1.91 (m, 2H), 1.19 (br s, 3H); 461.6 |
| 36 | 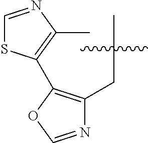 [SMILES: *Cc1ncoc1-c1scnc1C] | D | B; P1¹⁴ | (5R,7S)-1-(3- fluorophenyl)-7- methyl-8-{[5-(4- methyl-1,3-thiazol- 5-yl)-1,3-oxazol-4- yl]methyl}-2-thia- 1,8- diazaspiro[4.5]dec- 3-ene 2,2-dioxide | 8.74 (s, 1H), 7.87 (s, 1H), 7.41 (td, J = 8.2, 6.3 Hz, 1H), 7.16-7.23 (m, 2H), 7.12 (dt, J = 9.2, 2.2 Hz, 1H), 7.02 (d, J = 7.2 Hz, 1H), 6.80 (d, J = 7.2 Hz, 1H), 3.64 (br d, J = 14 Hz, 1H), 3.47-3.55 (m, 1H), 2.87 (br s, 1H), 2.60 (br s, 1H), 2.50 (s, 3H), 2.39 (br s, 1H), 2.12 (dd, J = 13.9, 4.7 Hz, 1H), 1.93-2.01 (m, 1H), 1.85 (br s, 1H), 1.74 (br s, 1H), 1.10 (br d, J = 6 Hz, 3H); 475.6 |
| 37 | 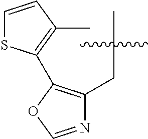 [SMILES: *Cc1ncoc1-c1sccc1C] | D | B; P1¹⁴ | (5R,7S)-1-(3- fluorophenyl)-7- methyl-8-{[5-(3- methyl-2-thienyl)- 1,3-oxazol-4- yl]methyl}-2-thia- 1,8- diazaspiro[4.5]dec- 3-ene 2,2-dioxide | 7.83 (s, 1H), 7.40 (td, J = 8.2, 6.3 Hz, 1H), 7.30 (d, J = 5.1 Hz, 1H), 7.16-7.22 (m, 2H), 7.11 (dt, J = 9.3, 2.2 Hz, 1H), 7.01 (d, J = 7.2 Hz, 1H), 6.88 (d, J = 5.1 Hz, 1H), 6.78 (d, J = 7.2 Hz, 1H), 3.68 (br d, J = 14 Hz, 1H), 3.54-3.63 (m, 1H), 2.81-2.91 (m, 1H), 2.59-2.67 (m, 1H), 2.39-2.46 (m, 1H), 2.24 (s, 3H), 2.12 (dd, J = 13.7, 4.5 Hz, 1H), 1.94-2.01 (m, 1H), 1.87 (br s, 1H), 1.76 (br s, 1H), 1.08 (d, J = 6.3 Hz, 3H); 474.6 |
| 38 | 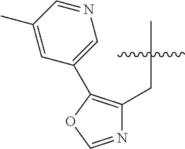 [SMILES: *Cc1ncoc1-c1cncc(C)c1] | D | B; P1¹⁴ | (5R,7S)-1-(3- fluorophenyl)-7- methyl-8-{[5-(5- methylpyridin-3- yl)-1,3-oxazol-4- yl]methyl}-2-thia- 1,8- diazaspiro[4.5]dec- 3-ene 2,2-dioxide | 8.70 (br s, 1H), 8.39 (br s, 1H), 7.86 (s, 1H), 7.79 (br s, 1H), 7.42 (td, J = 8.2, 6.4 Hz, 1H), 7.17-7.24 (m, 2H), 7.12 (dt, J = 9.3, 2.1 Hz, 1H), 7.06 (br d, J = 7 Hz, 1H), 6.81 (d, J = 7.2 Hz, 1H), 3.81 (br d, J = 13 Hz, 1H), 3.53 (br s, 1H), 2.94 (br s, 1H), 2.72 (br s, 1H), 2.44 (br s, 1H), 2.33 (s, 3H), 2.13 (dd, J = 13.8, 4.6 Hz, 1H), 1.93-2.02 (br s, 1H), 1.81 (br s, 2H), 1.19 (m, 3H); 469.6 |
| 39 | 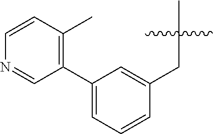 [SMILES: *Cc1cccc(-c2cnccc2C)c1] | D | A; P1¹⁵ | (5R,7S)-1-(3- fluorophenyl)-7- methyl-8-[3-(4- methylpyridin-3- yl)benzyl]-2-thia- 1,8- diazaspiro[4.5]dec- 3-ene 2,2-dioxide, hydrochloride salt | 8.54 (br d, J = 5 Hz, 1H), 8.49 (br s, 1H), 7.51-7.58 (m, 1H), 7.35-7.51 (m, 5H), 7.11-7.25 (m, 3H), 6.91 (br s, 2H), 4.49 (br d, J = 14 Hz, 1H), 3.69 (br d, J = 13 Hz, 1H), 3.14 (br d, J = 11 Hz, 1H), 2.81- 3.08 (m, 3H), 2.44 (s, 3H), 2.19-2.41 (m, 3H), 1.65 (br d, J = 6 Hz, 3H); 478.7 |
| 40 | 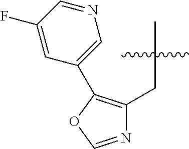 [SMILES: *Cc1ncoc1-c1cncc(F)c1] | D | B; P1¹⁴ | (5R,7S)-1-(3- fluorophenyl)-8- {[5-(5- fluoropyridin-3-yl)- 1,3-oxazol-4- yl]methyl}-7- methyl-2-thia-1,8- diazaspiro[4.5]dec- 3-ene 2,2-dioxide, hydrochloride salt | 13.26 (br s, 1H), 8.65 (br s, 1H), 8.59 (m, 1H), 7.90 (s, 1H), 7.62 (br d, J = 8 Hz, 1H), 7.44 (td, J = 8.1, 6.6 Hz, 1H), 7.24-7.29 (m, 1H), 7.14-7.23 (m, 2H), 6.89 (AB quartet, J_{AB} = 6.9 Hz, Δν_{AB} = 17.1 Hz, 2H), 4.56 (br d, J = 15 Hz, 1H), 4.22 (br d, J = 15 Hz, 1H), 3.39 (br s, 1H), 3.01-3.18 (m, 2H), 2.77-2.90 (m, 2H), 2.15-2.28 (m, 2H), 1.49 (d, J = 6.1 Hz, 3H); 473.6 |
| 41 | 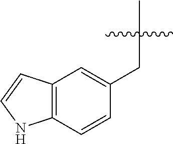 [SMILES: *Cc1ccc2[nH]ccc2c1] | D | A; P1 | (5R,7S)-1-(3- fluorophenyl)-8- (1H-indol-5- ylmethyl)-7- methyl-2-thia-1,8- diazaspiro[4.5]dec- 3-ene 2,2-dioxide | 8.33 (br s, 1H), 7.38 (s, 1H), 7.23-7.31 (m, 3H), 7.16-7.19 (m, 1H), 7.11-7.15 (m, 1H), 7.08 (dt, J = 9.2, 2.2 Hz, 1H), 7.03 (d, J = 7.2 Hz, 1H), 6.99 (dd, J = 8.2, 1.6 Hz, 1H), 6.76 (d, J = 7.2 Hz, 1H), 6.44-6.48 (m, 1H), 3.76 (d, J = 13.1 Hz, 1H), 3.45 (d, J = 13.1 Hz, 1H), 2.74-2.83 (m, 1H), 2.59-2.68 (m, 1H), 2.27 (ddd, J = 12.4, 8.0, 4.0 Hz, 1H), 2.11 (dd, J = 13.8, 4.4 Hz, 1H), 1.84-2.01 (m, 2H), 1.81 (dd, J = 13.9, 6.8 Hz, 1H), 1.17 (d, J = 6.6 Hz, 3H); 426.6 |
| 42 | 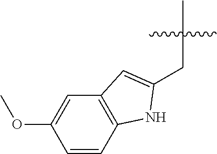 [SMILES: *Cc1cc2cc(OC)ccc2[nH]1] | D | A; P1 | (5R,7S)-1-(3- fluorophenyl)-8- [(5-methoxy-1H- indol-2-yl)methyl]- 7-methy1-2-thia- 1,8- diazaspiro[4.5]dec- 3-ene 2,2-dioxide | ¹H NMR (500 MHz, CDCl₃) δ 8.09 (br s, 1H), 7.21-7.26 (m, 2H), 7.12 (br d, J = 8.3 Hz, 1H), 7.01-7.10 (m, 3H), 6.95- 7.00 (m, 2H), 6.85 (dd, J = 8.8, 2.4 Hz, 1H), 6.79 (d, J = 7.3 Hz, 1H), 3.89 (d, J = 14.9 Hz, 1H), 3.74 (s, 3H), 3.62 (d, J = 13.7 Hz, 1H), 2.82-2.91 (m, 1H), 2.69-2.78 (m, 1H), 2.32-2.39 (m, 1H), 2.14 (dd, J = 13.9, 4.4 Hz, 1H), 1.93- 2.04 (m, 2H), 1.87 (br dd, J = 13.3, 6.7 Hz, 1H), 1.21 (d, J = 6.6 Hz, 3H); 456.6 |
| 43 | 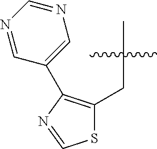 [SMILES: *Cc1scnc1-c1cncnc1] | D | A; P1¹⁷ | (5R,7S)-1-(3- fluorophenyl)-7- methyl-8-[(4- pyrimidin-5-yl-1,3- thiazol-5- yl)methyl]-2-thia- 1,8- diazaspiro[4.5]dec- 3-ene 2,2-dioxide | 9.17 (s, 1H), 9.00 (s, 2H), 8.80 (s, 1H), 7.46 (ddd, J = 8, 8, 6.3 Hz, 1H), 7.22- 7.27 (m, 1H), 7.19-7.22 (m, 1H), 7.14 (br ddd, J = 9, 2, 2 Hz, 1H), 7.05 (br d, J = 7.2 Hz, 1H), 6.83 (d, J = 7.2 Hz, 1H), 3.81 (AB quartet, J_{AB} =14.7 Hz, Δν_{AB} = 133 Hz, 2H), 2.88-2.97 (m, 1H), 2.67 (ddd, J = 12, 8,4 Hz, 1H), 2.32- 2.39 (m, 1H), 2.12 (dd, J = 13.8, 4.6 Hz, 1H), 1.92-2.00 (m, 1H), 1.75-1.89 (m, 2H), 1.11 (d, J = 6.4 Hz, 3H); 472.3 |
| 44 | 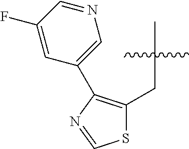 [SMILES: *Cc1scnc1-c1cncc(F)c1] | D | A; P1¹⁷ | (5R,7S)-1-(3- fluorophenyl)-8- {[4-(5- fluoropyridin-3-yl)- 1,3-thiazol-5- yl]methyl}-7- methyl-2-thia-1,8- diazaspiro[4.5]dec- 3-ene 2,2-dioxide, hydrochloride salt | 9.01 (s, 1H), 8.64 (d, J = 2.4 Hz, 1H), 8.40 (br s, 1H), 7.49-7.54 (m, 1H), 7.40 (ddd, J = 8, 8, 6 Hz, 1H), 7.21-7.26 (m, 1H), 7.12-7.16 (m, 1H), 7.07 (ddd, J = 9, 2, 2 Hz, 1H), 6.87 (AB quartet, upfield signal is broadened, J_{AB} = 7 Hz, Δν_{AB} = 20 Hz, 2H), 4.62 (br d, J = 14 Hz, 1H), 4.27 (br d, J = 14 Hz, 1H), 2.78- 2.99 (m, 3H), 2.72 (br d, J = 12 Hz, 1H), 2.23 (br d, J = 14 Hz, 1H), 2.03-2.16 (m, 2H), 1.47-1.54 (m, 3H); 489.3 |
| 45 | 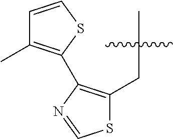 [SMILES: *Cc1scnc1-c1sccc1C] | D | A; P1¹⁷ | (5R,7S)-1-(3- fluorophenyl)-7- methyl-8-{[4-(3- methyl-2-thienyl)- 1,3-thiazol-5- yl]methyl}-2-thia- 1,8- diazaspiro[4.5]dec- 3-ene 2,2-dioxide | 8.74 (br s, 1H), 7.43 (ddd, J = 8.1, 8.1, 6.4 Hz, 1H), 7.28 (br d, J = 5.2 Hz, 1H), 7.17-7.24 (m, 2H), 7.13 (ddd, J = 9.3, 2.1, 2.1 Hz, 1H), 7.01-7.05 (m, 1H), 6.91 (d, J = 5.1 Hz, 1H), 6.80 (d, J = 7.1 Hz, 1H), 3.71 (br AB quartet, J_{AB} = 14 Hz, Δν_{AB} = 88 Hz, 2H), 2.81-2.90 (m, 1H), 2.56-2.64 (m, 1H), 2.34 (ddd, J = 12.5, 7.2, 3.9 Hz, 1H), 2.13 (s, 3H), 2.07-2.13 (m, 1H), 1.92-2.00 (m, 1H), 1.78-1.87 (m, 1H), 1.68-1.78 (m, 1H), 1.03 (br d, J = 6 Hz, 3H); 490.2 |
| 46 | 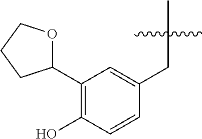 [SMILES: *Cc1ccc(O)c(C2CCCO2)c1] | S | Separation of diastereomers in Example 61; later- eluting isomer¹⁸ | 4-{[(5R,7S)-1-(3- fluorophenyl)-7- methyl-2,2- dioxido-2-thia-1,8- diazaspiro[4.5]dec- 8-yl]methyl}-2- (tetrahydrofuran-2- yl)phenol | ¹H NMR (500 MHz, CDCl₃) δ 8.49 (m, 1H), 7.32-7.38 (m, 1H), 7.08-7.17 (m, 2H), 7.05 (br d, J = 9 Hz, 1H), 6.95 (br d, J = 8 Hz, 1H), 6.80-6.86 (m, 1H), 6.77 (d, J = 8.2 Hz, 1H), 4.95 (dd, J = 9.2, 6.2 Hz, 1H), 4.10-4.16 (m, 1H), 3.93-3.99 (m, 1H), 3.48-3.71 (m, 1H), 3.25-3.39 (m, 3H), 2.70-2.78 (m, 1H), 2.62 (br s, 1H), 2.43-2.55 (m, 2H), 2.25-2.36 (m, 2H), 1.88-2.13 (m, 5H), 1.77 (br s, 2H), 1.08-1.22 (m, 3H); 475.1 |
| 47 | 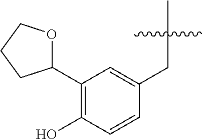 [SMILES: *Cc1ccc(O)c(C2CCCO2)c1] | S | Separation of diastereomers in Example 61; earlier- eluting isomer¹⁸ | 4-{[(5R,7S)-1-(3- fluorophenyl)-7- methyl-2,2- dioxido-2-thia-1,8- diazaspiro[4.5]dec- 8-yl]methyl}-2- (tetrahydrofuran-2- yl)phenol | ¹H NMR (500 MHz, CDCl₃) δ 8.42 (br s, 1H), 7.32-7.39 (m, 1H), 7.03-7.17 (m, 3H), 6.96 (br d, J = 8 Hz, 1H), 6.75-6.81 (m, 2H), 4.94 (dd, J = 9.0, 6.4 Hz, 1H), 4.09-4.16 (m, 1H), 3.93-3.99 (m, 1H), 3.45-3.58 (m, 1H), 3.25-3.38 (m, 3H), 2.73-2.79 (m, 1H), 2.39-2.59 (m, 3H), 2.23-2.35 (m, 2H), 1.86-2.11 (m, 5H), 1.45-1.82 (m, 2H), 1.02-1.20 (m, 3H); 475.1 |
| 48 | 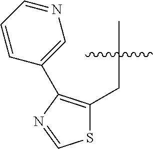 [SMILES: *Cc1scnc1-c1cccnc1] | D | A; P1¹⁷ | (5R,7S)-1-(3- fluorophenyl)-7- methyl-8-[(4- pyridin-3-yl-1,3- thiazol-5- yl)methyl]-2-thia- 1,8- diazaspiro[4.5]dec- 3-ene 2,2-dioxide | ¹H NMR (500 MHz, CDCl₃) δ 8.79 (br s, 2H), 8.59-8.63 (m, 1H), 7.93-7.99 (m, 1H), 7.42-7.47 (m, 1H), 7.40 (dd, J = 8, 5 Hz, 1H), 7.23 (ddd, J = 8, 8, 2 Hz, 1H), 7.20 (br d, J = 7.9 Hz, 1H), 7.11-7.14 (m, 1H), 7.01-7.05 (m, 1H), 6.82 (d, J = 7.1 Hz, 1H), 4.01 (br s, 1H), 3.70 (br s, 1H), 2.87-2.94 (m, 1H), 2.62-2.68 (m, 1H), 2.33 (br s, 1H), 2.13 (dd, J = 14, 4 Hz, 1H), 1.48-2.01 (m, 3H), 1.12 (br s, 3H); 471.2 |
| 49 | 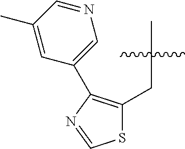 [SMILES: *Cc1scnc1-c1cncc(C)c1] | D | A; P1¹⁹ | (5R,7S)-1-(3- fluorophenyl)-7- methyl-8-{[4-(5- methylpyridin-3- yl)-1,3-thiazol-5- yl]methyl}-2-thia- 1,8- diazaspiro[4.5]dec- 3-ene 2,2-dioxide, hydrochloride salt | ¹H NMR (400 MHz, CD₃OD) δ 9.06 (s, 1H), 8.67 (br s, 1H), 8.48 (br s, 1H), 8.15 (br s, 1H), 7.51 (ddd, J = 8.2, 8.2, 6.6 Hz, 1H). 7.29-7.36 (m, 2H), 7.23 (ddd, J = 7.8, 1.9, 1.0 Hz, 1H), 7.19 (ddd, J = 9.5, 2.2, 2.2 Hz, 1H), 7.14 (d, J = 7.1 Hz, 1H), 4.12 (br AB quartet, J_{AB} = 14 Hz, Δν_{AB} = 105 Hz, 2H), 2.89- 2.99 (m, 1H), 2.75-2.84 (m, 1H), 2.47 (s, 3H), 2.30-2.40 (m, 1H), 2.12 (dd, J = 14.2, 3.7 Hz, 1H), 1.86-1.98 (m, 3H), 1.18 (d, J = 6.5 Hz, 3H); 485.2 |
| 50 | 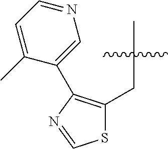 [SMILES: *Cc1scnc1-c1cnccc1C] | D | A; P1¹⁹ | (5R,7S)-1-(3- fluorophenyl)-7- methyl-8-{[4-(4- methylpyridin-3- yl)-1,3-thiazol-5- yl]methyl}-2-thia- 1,8- diazaspiro[4.5]dec- 3-ene 2,2-dioxide, hydrochloride salt | ¹H NMR (400 MHz, CD₃OD) δ 9.17 (s, 1H), 8.59 (d, J = 5.6 Hz, 1H), 8.49 (s, 1H), 7.68 (d, J = 5.6 Hz, 1H), 7.51 (ddd, J = 8.1, 8.1, 6.5 Hz. 1H), 7.32 (dddd, J = 8.4, 8.4, 2.5, 0.9 Hz, 1H), 7.20-7.27 (m, 3H), 7.16 (d, J = 7.1 Hz, 1H), 4.11- 4.24 (m, 1H), 3.88-4.03 (m, 1H), 2.78- 3.00 (m, 2H), 2.31 (s, 3H), 2.27-2.39 (m, 1H), 2.12-2.21 (m, 1H), 1.91-2.09 (m, 3H), 1.16 (br d, J = 6 Hz, 3H); 485.3 |
| 51 | 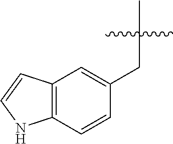 [SMILES: *Cc1ccc2[nH]ccc2c1] | S | C; Ex 41 | (5R,7S)-1-(3- fluorophenyl)-8- (1H-indol-5- ylmethyl)-7- methyl-2-thia-1,8- diazaspiro[4.5]dec ane 2,2-dioxide | 2.15²⁰; 428.1 |
| 52 | 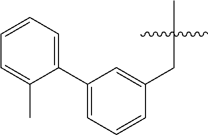 [SMILES: *Cc1cccc(-c2ccccc2C)c1] | S | C; Ex 54 | (5R,7S)-1-(3- fluorophenyl)-7- methyl-8-[(2′- methylbiphenyl-3- yl)methyl]-2-thia- 1,8- diazaspiro[4.5]dec ane 2,2-dioxide, formate salt | 2.78²⁰; 479.1 |
| 53 | 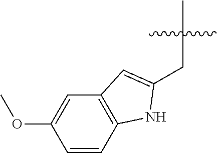 [SMILES: *Cc1cc2cc(OC)ccc2[nH]1] | S | C; Ex 42 | (5R,7S)-1-(3- fluorophenyl)-8- [(5-methoxy-1H- indol-2-yl)methyl]- 7-methyl-2-thia- 1,8- diazaspiro[4.5]dec ane 2,2-dioxide | 2.15²⁰; 458.1 |
| 54 | 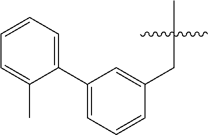 [SMILES: *Cc1cccc(-c2ccccc2C)c1] | D | A; P1 | (5R,7S)-1-(3- fluorophenyl)-7- methyl-8-[(2′- methylbiphenyl-3- yl)methyl]-2-thia- 1,8- diazaspiro[4.5]dec- 3-ene 2,2-dioxide | ¹H NMR (500 MHz, CDCl₃) δ 7.42 (ddd, J = 8.1, 8.1, 6.5 Hz, 1H), 7.29-7.33 (m, 1H), 7.13-7.27 (m, 10H), 7.09 (d, J = 7.2 Hz, 1H), 6.80 (d, J = 7.2 Hz, 1H), 3.72 (d, J = 13.4 Hz, 1H), 3.33 (d, J = 13.3 Hz, 1H), 2.80-2.87 (m, 1H), 2.63-2.69 (m, 1H), 2.28-2.33 (m, 1H), 2.23 (s, 3H), 2.15 (dd, J = 13.8, 4.6 Hz, 1H), 2.00 (ddd, J = 14, 8, 4 Hz, 1H), 1.83-1.89 (m, 1H), 1.77 (br dd, J = 14, 6 Hz, 1H), 1.14 (d, J = 6.5 Hz, 3H); 477.1 |
| 55 | 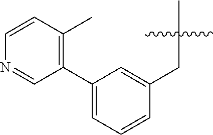 [SMILES: *Cc1cccc(-c2cnccc2C)c1] | S | A; P4¹⁵ | (5R,7S)-1-(3- fluorophenyl)-7- methyl-8-[3-(4- methy\|pyridin-3- yl)benzyl]-2-thia- 1,8- diazaspiro[4.5]dec ane 2,2-dioxide, hydrochloride salt | 8.53 (br s, 1H), 8.44 (br s, 1H), 7.52- 7.58 (m, 1H), 7.30-7.47 (m, 5H), 7.08- 7.16 (m, 2H), 7.01 (br d, J = 9 Hz, 1H), 4.44 (br d, J = 13 Hz, 1H), 3.79 (br d, J = 13 Hz, 1H), 3.43-3.49 (m, 2H), 3.09 (br d, J = 11 Hz, 1H), 2.70-2.92 and 2.35-2.54 (2 multiplets, 8H), 2.41 (s, 3H), 1.61-1.67 (m, 3H); 480.7 |
| 56 | 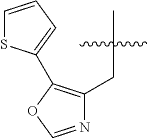 [SMILES: *Cc1ncoc1-c1cccs1] | S | B; P4¹⁴ | (5R,7S)-1-(3- fluorophenyl)-7- methyl-8-{[5-(2- thienyl)-1,3- oxazol-4- yl]methyl}-2-thia- 1,8- diazaspiro[4.5]dec ane 2,2-dioxide, hydrochloride salt | 12.92 (br s, 1H), 7.82 (br s, 1H), 7.51 (dd, J = 5.1, 1.1 Hz, 1H), 7.31-7.37 (m, 1H), 7.31 (dd, J = 3.7, 1.1 Hz, 1H), 7.16 (dd, J = 5.1, 3.7 Hz. 1H), 7.14-7.19 (m, 1H), 7.07 (ddd, J = 7.8, 1.9, 0.9 Hz, 1H), 7.03 (ddd, J = 8.9, 2.2, 2.2 Hz, 1H), 4.48 (br d, J = 15.4. 2.4 Hz, 1H), 4.11 (br d, J = 15.4 Hz, 1H), 3.42-3.47 (m, 2H), 3.02-3.17 (m, 2H), 2.65-2.85 (m, 2H), 2.54 (dd, J = 15.2, 12.6 Hz, 1H), 2.38- 2.49 (m, 3H), 2.29-2.36 (m, 1H), 1.51 (d, J = 6.2 Hz, 3H); 462.6 |
| 57 | 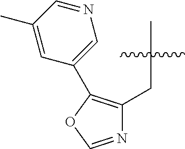 [SMILES: *Cc1ncoc1-c1cncc(C)c1] | S | B; P4¹⁴ | (5R,7S)-1-(3- fluorophenyl)-7- methyl-8-{[5-(5- methylpyridin-3- yl)-1,3-oxazol-4- yl]methyl}-2-thia- 1,8- diazaspiro[4.5]dec ane 2,2-dioxide, hydrochloride salt | 12.92 (br s, 1H), 8.67 (br s, 1H), 8.53 (br s, 1H), 7.92 (s, 1H), 7.70 (br s, 1H), 7.36-7.43 (m, 1H), 7.21 (br ddd, J = 8, 8, 2 Hz, 1H), 7.07-7.13 (m, 2H), 4.43 (br AB quartet, J_{AB} = 15 Hz, Δν_{AB} = 140 Hz, 2H), 3.42-3.47 (m, 2H), 3.27-3.38 (m, 1H), 3.04-3.12 (m, 1H), 2.71-2.83 (m, 2H), 2.48 (s, 3H), 2.39-2.60 (m, 4H), 2.29-2.36 (m, 1H), 1.47 (d, J = 6.2 Hz, 3H); 471.7 |
| 58 | 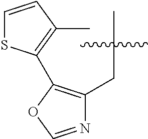 [SMILES: *Cc1ncoc1-c1sccc1C] | S | B; P4¹⁴ | (5R,7S)-1-(3- fluorophenyl)-7- methyl-8-{[5-(3- methyl-2-thienyl)- 1,3-oxazol-4- yl]methyl}-2-thia- 1,8- diazaspiro[4.5]dec ane 2,2-dioxide, hydrochloride salt | 12.84 (br s, 1H), 7.91 (br s, 1H), 7.44 (d, J = 5.0 Hz, 1H), 7.38 (ddd, J = 8.2, 8.2, 6.4 Hz, 1H), 7.17 (br ddd, J = 8.2, 8.2, 2.4 Hz, 1H), 7.09-7.12 (m, 1H), 7.06 (ddd, J = 9.2, 2.3, 2.2 Hz, 1H), 6.98 (d, J = 5.2 Hz, 1H), 4.39 (br dd, J = 15.1, 2.2 Hz, 1H), 3.91 (br d, J = 15.2 Hz, 1H), 3.42-3.47 (m, 2H), 3.02-3.12 (m, 1H), 2.92-2.98 (m, 1H), 2.66-2.85 (m, 2H), 2.53 (dd, J = 15.1, 12.6 Hz, 1H), 2.30- 2.49 (m, 4H), 2.19 (s, 3H), 1.41 (d, J = 6.2 Hz, 3H); 476.6 |
| 59 | 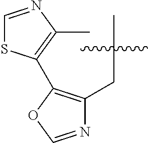 [SMILES: *Cc1ncoc1-c1scnc1C] | S | B; P4¹⁴ | (5R,7S)-1-(3- fluorophenyl)-7- methyl-8-{[5-(4- methyl-1,3-thiazol- 5-yl)-1,3-oxazol-4- yt]methyl}-2-thia- 1,8- diazaspiro[4.5]dec ane 2,2-dioxide, hydrochloride salt | 13.04 (br s, 1H), 8.90 (s, 1H), 7.96 (br s, 1H), 7.38 (ddd, J = 8.1, 8.1, 6.5 Hz, 1H), 7.18 (br ddd, J = 8.2, 8.1, 2.5 Hz, 1H), 7.10-7.13 (m, 1H), 7.07 (ddd, J = 9.1, 2.2, 2.2 Hz, 1H), 4.37 (br d, J = 15.2 Hz, 1H), 3.89 (br d, J = 15.3 Hz, 1H), 3.43-3.48 (m, 2H), 3.09-3.20 (m, 1H), 2.92-2.99 (m, 1H), 2.65-2.86 (m, 2H), 2.54 (dd, J = 15.1, 12.5 Hz, 1H), 2.45 (s, 3H), 2.31-2.50 (m, 4H), 1.44 (d, J = 6.2 Hz, 3H); 477.6 |
| 60 | 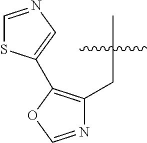 [SMILES: *Cc1ncoc1-c1cncs1] | S | B; P4¹⁴ | (5R,7S)-1-(3- fluorophenyl)-7- methyl-8-{[5-(1,3- thiazol-5-yl)-1,3- oxazol-4- yl]methyl}-2-thia- 1,8- diazaspiro[4.5]dec ane 2,2-dioxide, hydrochloride salt | 13.13 (br s, 1H), 8.97 (s, 1H), 8.13 (s, 1H), 7.90 (s, 1H), 7.35 (ddd, J = 8.1, 8.1, 6.4 Hz, 1H), 7.17 (dddd, J = 8.2, 8.2, 2.4, 0.8 Hz, 1H), 7.07-7.10 (m, 1H), 7.04 (ddd, J = 9.0, 2.2, 2.2 Hz, 1H), 4.46 (br dd, J = 15.4, 2.3 Hz, 1H), 4.06 (br d, J = 15.3 Hz, 1H), 3.43-3.48 (m, 2H), 3.13-3.24 (m, 1H), 3.02-3.08 (m, 1H), 2.65-2.87 (m, 2H), 2.55 (dd, J = 15.1, 12.5 Hz, 1H), 2.40-2.51 (m, 3H), 2.33 (br d, J = 14.6 Hz, 1H), 1.52 (d, J = 6.2 Hz, 3H); 463.6 |
| 61 | 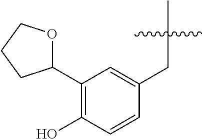 [SMILES: *Cc1ccc(O)c(C2CCCO2)c1] | S | A; P4⁵ | 4-{[(5R,7S)-1-(3- fluorophenyl)-7- methyl-2,2- dioxido-2-thia-1,8- diazaspiro[4.5]dec- 8-yl]methyl}-2- (tetrahydrofuran-2- yl)phenol (mixture of diastereomers at the tetrahydrofuran substituent) | ¹H NMR (500 MHz, CDCl₃) δ 8.40 (br s, 1H), 7.34-7.40 (m, 1H), 7.12-7.18 (m, 2H), 7.05-7.09 (m, 1H), 6.36 (ddd, J = 8.3, 2.0, 2.0 Hz, 1H), 6.74-6.80 (m, 2H), 4.92-4.96 (m, 1H), 4.10-4.15 (m, 1H), 3.93-3.98 (m, 1H), 3.46-3.54 (m, 1H), 3.31-3.37 (m, 2H), 3.23-3.30 (m. 1H), 2.70-2.80 (m, 1H), 2.43-2.57 (m, 3H), 2.25-2.34 (m, 2H), 2.00-2.10 (m, 3H), 1.89-1.99 (m, 2H), 1.56-1.77 (m, 2H), 1.08 and 1.09 (2 doublets, J = 6.7 Hz, 3H); 475.2 |
| 62 | 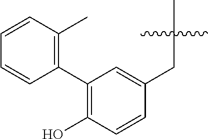 [SMILES: *Cc1ccc(O)c(-c2ccccc2C)c1] | S | C; Ex 25 | 5-{[(5R,7S)-1-(3- fluorophenyl)-7- methyl-2,2- dioxido-2-thia-1,8- diazaspiro[4.5]dec- 8-yl]methyl-2′- methylbiphenyl-2- ol, hydrochloride salt | ¹H NMR (400 MHz, CDCl₃ + drop of D₂O) δ 7.21-7.40 (m, 4H), 7.07-7.20 (m, 3H), 6.93-7.05 (m, 4H), 4.03-4.22 (m, 1H), 3.84-4.03 (m, 1H), 3.42 (t, J = 7.6 Hz, 2H), 2.96-3.13 (m, 1H), 2.55- 2.84 (m, 3H), 2.46 (t, J = 7.6 Hz, 2H), 2.27-2.49 (m, 3H), 2.14 (s, 3H), 1.51- 1.65 (m, 3H); 495.7 |
| 69 | 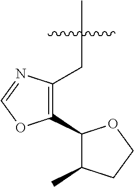 [SMILES: *Cc1ncoc1[C@H]1OCC[C@H]1C] | D | A; P1²¹ | (5R,7S)-1-(3- fluorophenyl)-7- methy1-8-({5-[cis-3- methyltetrahydrofuran- 2-yl]-1,3-oxazol- 4-yl}methyl)-2-thia- 1,8- diazaspiro[4.5]dec- 3-ene 2,2-dioxide | 0.76 (d, J = 7.0 Hz, 3H), 1.15 (d, J = 6.4 Hz, 3H), 1.75 (dd, J = 13.8, 6.7 Hz, 1H), 1.82-1.92 (m, 2H), 1.97-2.19 (m, 3H), 2.29-2.43 (m, 1H), 2.45-2.55 (m, 1H), 2.59-2.69 (m, 1H), 2.83 (td, J = 12.8, 6.4 Hz, 1H), 3.39 (dd, J = 19.3, 13.9 Hz, 1H), 3.53-3.63 (m, 1H), 3.78-3.87 (m, 1H), 4.10 (tt, J = 8.2, 2.8 Hz, 1H), 4.99 (dd, J = 7.5, 2.6 Hz, 1H), 6.79 (d, J = 7.2 Hz, 1H), 7.02 (dd, J = 10.2, 7.0 Hz, 1H), 7.10-7.16 (m, 1H), 7.16-7.23 (m, 2H), 7.33-7.44 (m, 1H), 7.72 (s, 1H); 462.1 |
| 70 | 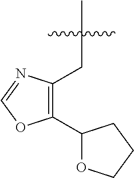 [SMILES: *Cc1ncoc1C1CCCO1] | D | A; P1²¹ | (5R,7S)-1-(3- fluorophenyl)-7- methyl-8-{[5- (tetrahydrofuran-2- yl)-1,3-oxazol-4- yl]methyl}-2-thia- 1,8- diazaspiro[4.5]dec- 3-ene 2,2-dioxide | ¹H NMR (500 MHz, CD₃OD) δ 1.23 (overlapping doublet, J = 7.6, 6.4 Hz, 3H), 1.84 (dd, J = 14.0, 9.4 Hz, 1H), 1.99- 2.29 (m, 7.5H), 2.43 (ddd, J = 13.0, 9.3, 4.0 Hz, 0.5H), 2.61-2.79 (m, 2H), 3.63- 3.71 (m, 2H), 3.80-3.88 (m, 1H), 3.92- 4.02 (m, 1H), 4.94-5.02 (m, 1H), 7.09- 7.12 (m, 1H), 7.15-7.32 (m, 4H), 7.46 (tdd, J = 8.2, 6.5, 3.5 Hz, 1H), 8.02 (overlapping singlets, 1H); 448.3 |
| 71 | 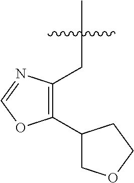 [SMILES: *Cc1ncoc1C1CCOC1] | D | A; P1²¹ | (5R,7S)-1-(3- fluorophenyl)-7- methyl-8-{[5- (tetrahydrofuran-3- yl)-1,3-oxazol-4- yl]methyl}-2-thia- 1,8- diazaspiro[4.5]dec- 3-ene 2,2-dioxide | ¹H NMR (400 MHz, CD₃OD) δ 7.95 (s, 1H), 7.41 (td, J = 8.1, 6.4 Hz, 1H), 7.06- 7.26 (m, 5H), 3.79-4.02 (m, 3H), 3.50- 3.73 (m, 4H), 2.64-2.84 (m, 2H), 1.91- 2.35 (m, 6H), 1.80-1.91 (m, 1H), 1.23 (d, J = 6.2 Hz, 3H); 448.3 |
| 72 | 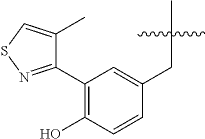 [SMILES: *Cc1ccc(O)c(-c2nscc2C)c1] | D | A; P1²² | 4-{[(5R,7S)-1-(3- fluorophenyl)-7- methyl-2,2-dioxido- 2-thia-1,8- diazaspiro[4.5]dec- 3-en-8-yl]methyl}- 2-(4-methyl-1,2- thiazol-3-yl)phenol, trifluoroacetic acid salt | 2.10 min²⁰; 500.2 |
| 73 | 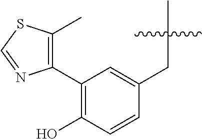 [SMILES: *Cc1ccc(O)c(-c2ncsc2C)c1] | D | A; P1²³ | 4-{[(5R,7S)-1-(3- fluorophenyl)-7- methyl-2,2-dioxido- 2-thia-1,8- diazaspiro[4.5]dec- 3-en-8-yl]methyl}- 2-(5-methyl-1,3- thiazol-4-yl)phenol, ammonium sail | 2.01 min²⁰; 500.2 |
| 74 | 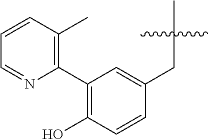 [SMILES: *Cc1ccc(O)c(-c2ncccc2C)c1] | D | A; P1²⁴ | 4-{[(5R,7S)-1-(3- fluorophenyl)-7- methyl-2,2-dioxido- 2-thia-1,8- diazaspiro[4.5]dec- 3-en-8-yl]methyl}- 2-(3-methylpyridin- 2-yl)phenol | 8.44 (dd, J = 4.7, 1.4 Hz, 1H), 7.69 (dd, J = 7.7, 0.9 Hz, 1H), 7.35-7.43 (m, 2H), 7.16-7.24 (m, 3H), 7.13 (dt, J = 9.5, 2.1 Hz, 1H), 7.06-7.11 (m, 2H), 6.97 (d, J = 8.4 Hz, 1H), 6.80 (d, J = 7.2 Hz, 1H), 3.61 (d, J = 13.3 Hz, 1H), 3.35 (d, J = 13.5 Hz, 1H), 2.81-2.88 (m, 1H), 2.59- 2.67 (m, 1H), 2.44 (s, 3H), 2.32 (ddd, J = 12.2, 7.6, 4.0 Hz, 1H), 2.13 (dd, J = 13.8, 4.8 Hz, 1H), 1.95-2.03 (m, 1H), 1.84 (br s, 1H), 1.76 (dd, J = 13.7, 5.5 Hz, 1H), 1.14 (d, J = 6.4 Hz, 3H); 494.1 |
| 75 | 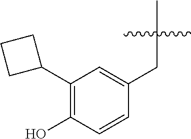 [SMILES: *Cc1ccc(O)c(C2CCC2)c1] | D | A; P1⁴ | 2-cyclobutyl-4- {[(5R,7S)-1-(3- fluorophenyl)-7- methyl-2,2-dioxido- 2-thia-1,8- diazaspiro[4.5]dec- 3-en-8- yl]methyl}phenol | 7.41 (td, J = 8.0, 6.5 Hz, 1H), 7.20 (dd, J = 8.3, 2.0 Hz, 2H), 7.11-7.18 (m, 1H), 7.08 (d, J = 7.2 Hz, 1H), 6.93-6.95 (m, 1H), 6.87-6.91 (m, 1H), 6.79 (d, J = 7.2 Hz, 1H), 6.65 (d, J = 8.2 Hz, 1H), 4.55 (br s, 1H), 3.59-3.67 (m, 1H), 3.59 (d, J = 13.1 Hz, 1H), 3.26 (d, J = 13.3 Hz, 1H), 2.73-2.82 (m, 1H), 2.60 (ddd, J = 12.3, 8.3, 3.9 Hz, 1H), 2.30-2.40 (m, 2H), 2.22-2.29 (m, 1H), 2.04-2.17 (m, 3H), 1.99 (ddd, J = 17.2, 8.0, 3.8 Hz, 2H), 1.80-1.90 (m, 2H), 1.76 (dd, J = 13.9, 6.2 Hz, 1H), 1.13 (d, J = 6.6 Hz, 3H): 457.1 |
| 76 | 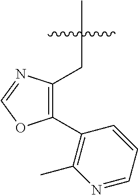 [SMILES: *Cc1ncoc1-c1cccnc1C] | D | A; P1²¹ | (5R,7S)-1-(3- fluorophenyl)-7- methyl-8-{[5-(2- methylpyridin-3-yl)- 1,3-oxazol-4- yl]methyl}-2-thia- 1,8- diazaspiro[4.5]dec- 3-ene 2,2-dioxide | 8.51 (dd, J = 4.9, 1.6 Hz, 1H), 7.89 (s, 1H), 7.58 (dd, J = 7.8, 1.8 Hz, 1H), 7.42 (td, J = 8.2, 6.4 Hz, 1H), 7.15-7.24 (m, 2H), 7.08-7.14 (m, 2H), 6.98 (d, J = 7.2 Hz, 1H), 6.78 (d, J = 7.2 Hz, 1H), 3.58 (d, J = 14.0 Hz, 1H), 3.42 (d, J = 14.2 Hz, 1H), 2.76-2.86 (m, 1H), 2.54-2.63 (m, 1H), 2.47 (s, 3H), 2.32-2.40 (m, 1H), 2.00-2.07 (m, 1H), 1.84-1.92 (m, 1H), 1.74-1.82 (m, 1H), 1.66-1.73 (m, 1H), 1.01 (d, J = 6.4 Hz, 3H); 469.2 |
| 77 | 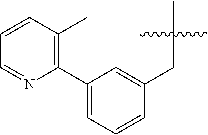 [SMILES: *Cc1cccc(-c2ncccc2C)c1] | D | A; P1²⁵ | (5R,7S)-1-(3- fluorophenyl)-7- methyl-8-[3-(3- methylpyridin-2- yl)benzyl]-2-thia- 1,8- diazaspiro[4.5]dec- 3-ene 2,2-dioxide, trifluoroacetic acid salt | 1.71 min²⁰; 478.3 |
| 78 | 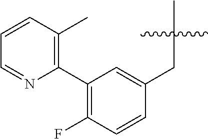 [SMILES: *Cc1ccc(F)c(-c2ncccc2C)c1] | D | A; P1²⁶ | (5R,7S)-8-[4- fluoro-3-(3- methylpyridin-2- yl)benzyl]-1-(3- fluorophenyl)-7- methyl-thia-1,8- diazaspiro[4.5]dec- 3-ene 2,2-dioxide | 1.95 min²⁰; 496.3 |
| 79 | 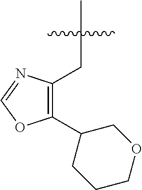 [SMILES: *Cc1ncoc1C1CCCOC1] | D | A; P1²¹ | (5R,7S)-1-(3- fluorophenyl)-7- methyl-8-{[5- (tetrahydro-2H- pyran-3-yl)-1,3- oxazol-4- yl]methyl}-2-thia- 1,8- diazaspiro[4.5]dec- 3-ene 2,2-dioxide | 2.08 min²⁰; 462.1 |
| 80 | 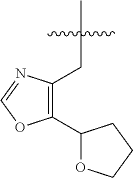 [SMILES: *Cc1ncoc1C1CCCO1] | S | A; P4²¹ | (5R,7S)-1-(3- fluorophenyl)-7- methyl-8-{[5- (tetrahydrofuran-2- yl)-1,3-oxazol-4- yl]methyl}-2-thia- 1,8- diazaspiro[4.5]dec ane 2,2-dioxide | mixture of diastereomers; 1.12 and 1.14 (2 d, J = 6.8 Hz, 3H), 1.61-1.68 (m, 1H), 1.72-1.83 (m, 1H), 1.91-2.16 (m, 6H), 2.30-2.64 (m, 4H), 2.74-2.87 (m, 1H), 3.30-3.39 (m, 2H), 3.42-3.58 (m, 2H), 3.78-3.86 (m, 1H), 3.93-4.02 (m, 1H), 4.93 (q, J = 6.6 Hz, 1H), 7.02- 7.16 (m, 3H), 7.35 (tdd, J = 8.1, 6.5, 1.8 Hz, 1H), 7.71 (s, 1H); 450.4 |

1. The phenolic hydroxy group of 3-bromo-4-hydroxybenzaldehyde was protected to provide 3-bromo-4-[(2-methoxyethoxy)methoxy]benzaldehyde. Suzuki reaction with (3-methyl-2-thienyl)boronic acid, followed by acidic deprotection, afforded the requisite aldehyde.
2. NMR data was obtained on the free base, prior to formation of the hydrochloride salt.
3. The requisite aldehyde was prepared by Suzuki reaction of 3-bromo-4-methoxybenzaldehyde with the appropriate boronic acid, followed by boron tribromide-mediated cleavage of the methoxy group.
4. Methyl 3-iodo-4-methoxybenzoate was treated with isopropylmagesium chloride and cyclo-pentanone; the resulting alcohol was reductively removed with trifluoroacetic and triethylsilane. Reduction of the methyl ester with lithium aluminum hydride was followed by oxidation to the aldehyde with Dess-Martin reagent. Boron tribromide-mediated cleavage of the methoxy group provided the requisite aldehyde.
5. 2-(Tetrahydrofuran-2-yl)phenol (prepared according to the method of J. T. Pinhey and P. T. Xuan, Aust. J. Chem. 1988, 41, 69-80) was brominated with N-bromosuccinimide. Metal-halogen exchange and reaction with N,N-dimethylformamide yielded the requisite aldehyde.
6. The corresponding ethyl 5-substituted-1,3-oxazole-4-carboxylate was prepared from ethyl isocyanoacetate and the appropriate acid chloride in the presence of base, according to the method of W. L. F. Armarego et al., Eur. J. Med. Chem. 1987, 22, 283-91. Diisobutylaluminum hydride reduction of the ester providied the requisite aldehyde.
7. 2-(Cyclopropyloxy)phenol (prepared by the method of P. D. O'Shea et al., J. Org. Chem. 2005, 70, 3021-3030) was brominated with N-bromosuccinimide. Metal-halogen exchange and reaction with N,N-dimethylformamide yielded the requisite aldehyde.
8. The corresponding alcohol was converted to the requisite bromide with phosphorus tribromide.
9. Cyclobutylacetic acid was converted to ethyl 4-cyclobutyl-3-oxobutanoate by reaction with 2,2-dimethyl-1,3-dioxane-4,6-dione followed by hydrolysis and decarboxylation. Chlorination with sulfuryl chloride and reaction with thioformamide provided ethyl 4-(cyclobutylmethyl)-1,3-thiazole-5-carboxylate, which was reduced with diisobutylaluminum hydride to the alcohol, and oxidized to the requisite aldehyde with activated manganese(IV) oxide.
10. The aldehyde was prepared as in footnote 9, except that 3-methylbutanoic acid was used in place of cyclobutylacetic acid, and formamide instead of thioformamide.
11. Nicotinic acid was converted to its acid chloride. Reaction with ethyl isocyanoacetate in the presence of base provided the oxazole; reduction of the ethyl ester with sodium borohydride followed by Dess-Martin oxidation gave the aldehyde.
12. The aldehyde was prepared as in footnote 11, except that pyrimidine-5-carboxylic acid was used in place of nicotinic acid, lithium aluminum hydride was used instead of sodium borohydride, and the final oxidation employed manganese(IV) oxide.
13.The aldehyde was prepared as in footnote 9 except that cyclopropylacetic acid was used as starting material.
14.The corresponding methyl 5-substituted-1,3-oxazole-4-carboxylate was prepared from methyl isocyanoacetate and the appropriate acid chloride in the presence of base. Diisobutylaluminum hydride reduction of the ester gave the corresponding alcohol, which was converted to the requisite chloride with thionyl chloride.
15. 4-Methylpyridin-3-amine was diazotized and then iodinated with potassium iodide. The iodide was converted to the corresponding boronic acid by treatment with n-butyllithium and tripropyl borate; Suzuki reaction with 3-bromobenzaldehyde provided the requisite aldehyde.
16. Chromatographic separation: Chiralcel OJ-H column, 5 μm (Mobile phase: 80/20 CO₂/methanol with 0.2% isopropylamine).
17. 4-Bromo-1,3-thiazole-5-carbaldehyde was prepared by manganese(IV) oxide oxidation of the corresponding alcohol. Suzuki reaction with the appropriate boronic acid gave the required aldehyde.
18. Chromatographic separation: Chiralpak AD-H column, 5 μm (Mobile phase: 70/30 CO₂/ethanol with 0.2% isopropylamine).
19. (4-Bromo-1,3-thiazol-5-yl)methanol was protected as its tert-butyl(dimethyl)silyl ether and subjected to a Suzuki reaction with the appropriate boronic acid. Fluoride-mediated deprotection was followed by manganese(IV) oxide oxidation to provide the requisite aldehyde.
20. HPLC conditions. Column: Waters Atlantis dC₁₈, 4.6 x 50 mm, 5 μm; Mobile phase A: 0.05% TFA in water (v/v); Mobile phase B: 0.05% TFA in acetonitrile (v/v); Gradient: 5% to 95% B over 4.0 min (linear gradient); Flow rate: 2.0 mL/min.
21. The corresponding 5-substituted-1,3-oxazole-4-carboxylate ester was prepared from methyl or ethyl isocyanoacetate and the appropriate acid chloride in the presence of base. Lithium triethylborohydride or sodium borohydride reduction of the ester gave the corresponding alcohol, which was converted to the requisite aldehyde using Dess-Martin reagent or manganese(IV) oxide.
22. Condensation of thioacetic acid with methacrylic acid followed by de-acetylation provided 3-mercapto-2-methylpropanoic acid, which was converted to the corresponding 3,3′-disulfanediylbis(2-methylpropanamide) by treatment with sodium hydroxide, followed by thionyl chloride and aqueous ammonia. Condensation of this disulfide with thionyl chloride afforded 4-methylisothiazol-3(2H)-one. Bromination of this thiazole with phosphorus oxybromide yielded
3-bromo-4-methylisothiazole. Suzuki coupling with 4-[(2-methoxyethoxy)methoxy]-3-(4,4,5,5-tetramethyl-1,3,2-dioxaborolan-2-yl)benzaldehyde followed by treatment with hydrochloric acid afforded the desired aldehyde.
23. Bromination of 5-methylthiazole afforded 4-bromo-5-methylthiazole, which was coupled to 4-[(2-methoxyethoxy)methoxy]-3-(4,4,5,5-tetramethyl-1,3,2-dioxaborolan-2-yl)benzaldehyde via Suzuki coupling. Deprotection of the phenol with hydrochloric acid yielded the desired aldehyde.
24. The phenolic hydroxy group of 3-bromo-4-hydroxybenzaldehyde was protected to provide 3-bromo-4[(2-methoxyethoxy)methoxy]benzaldehyde. The bromide was converted to the corresponding pinacol ester by reaction with bis(pinacolato)diboron; Suzuki reaction with 2-bromo-3-methylpyridine followed by acid deprotection afforded the requisite aldehyde.
25. Suzuki coupling of 3-formylphenylboronic acid pinacol ester and 2-bromo-3-methylpyridine provided the requisite aldehyde.
26. A Suzuki coupling was performed between 2-fluoro-5-formylphenyl boronic acid and 2-bromo-3-methylpyridine.

TABLE 12
Examples 63-66 and 81

| | |
|---|---|
| 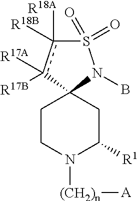 | A = 3-isopropoxyphenyl B = 3-fluorophenyl n = 1 R¹ = CH₃ |
| 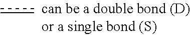 | |

| Ex # | Structure | 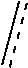 | Method of preparation; starting material(s) | IUPAC Name | ¹H NMR (400 MHz, CDCl₃), δ (ppm); Mass spectrum, observed ion m/z (M + 1) or HPLC retention time (minutes); Mass spectrum m/z (M + 1) |
|---|---|---|---|---|---|
| 63 | R^{17A} = methoxy R^{18A} = H | D | B; P6¹ | (5R,7S)-1-(3- fluorophenyl)-8-(3- isopropoxybenzyl)-4- methoxy-7-methyl-2- thia-1,8- diazaspiro[4.5]dec-3- ene 2,2-dioxide | 7.32-7.38 (m, 1H), 7.22- 7.27 (m, 1H), 7.12-7.20 (m, 3H), 6.69-6.76 (m, 3H), 5.81 (s, 1H), 4.52 (septet, J = 6.0 Hz, 1H), 3.86 (s, 3H), 3.83 (d, J = 13.7 Hz, 1H), 2.88 (d, J = 13.5 Hz, 1H), 2.53 (ddd, J = 12, 5, 2 Hz, 1H), 2.22-2.31 (m, 1H), 1.93-2.17 (m, 4H), 1.77 (ddd, J = 13, 13, 2 Hz, 1H), 1.33 (d, J = 6.0 Hz, 6H), 1.07 (d, J = 5.6 Hz, 3H); 475.2 |
| 64 | R^{17A} and R^{17B} together are C═O R^{18A} = methyl R^{18B} = methyl | S | B; P7¹ | (5R,7S)-1-(3- fluorophenyl)-8-(3- isopropoxybenzyl)- 3,3,7-trimethyl-2-thia- 1,8- diazaspiro[4.5]decan-4- one 2,2-dioxide, hydrochloride salt | 7.36-7.42 (m, 1H), 7.24- 7.27 (m, 1H), 7.12-7.19 (m, 3H), 6.69-6.75 (m, 3H), 4.52 (septet, J = 6.0 Hz, 1H), 3.81 (d, J = 13.7 Hz, 1H), 2.89 (d, J = 13.6 Hz, 1H), 2.56 (ddd, J = 12.4, 4.8, 3.2 Hz, 1H), 2.01-2.25 (m, 5H), 1.75 (ddd, J = 12.5, 12.5, 3.1 Hz, 1H), 1.61 (s, 3H), 1.59 (s, 3H), 1.33 (d, J = 6.0 Hz, 6H), 1.07 (d, J = 5.8 Hz, 3H);² 489.0 |
| 65 | R^{17A} = OH R^{17B} = H R^{18A} = H R^{18B} = H | S | A; P3 | (5R,7S)-1-(3- fluorophenyl)-8-(3- isopropoxybenzyl)-7- methyl-2-thia-1,8- diazaspiro[4.5]decan-4- ol 2,2-dioxide | 2.34;³ 463.7 |
| 66 | R^{17A} = NHMe R^{18A} = H | D | A; P5 | (5R,7S)-1-(3- fluorophenyl)-8-(3- isopropoxybenzyl)-N,7- dimethyl-2-thia-1,8- diazaspiro[4.5]dec-3- en-4-amine 2,2-dioxide | 2.39;³ 474.2 |
| 81 | R^{17A} and R^{17B} together are C═O R^{18A} = H R^{18B} = H | S | Example 63⁴ | (5R,7S)-1-(3- fluorophenyl)-8-(3- isopropoxybenzyl)-7- methyl-2-thia-1,8- diazaspiro[4.5]decan-4- one 2,2-dioxide | 1.89 min⁵; 461.2 |

1. The corresponding alcohol was converted to the requisite bromide with phosphorus tribromide.
2. NMR data was obtained on the free base, prior to formation of the hydrochloride salt.
3. HPLC conditions: Column: Waters Atlantis dC₁₈, 4.6 × 50 mm, 5 μm; Mobile phase A: 0.05% TFA in water (v/v); Mobile phase B: 0.05% TFA in acetonitrile (v/v); Gradient: 5% to 95% B over 4.0 min (linear gradient), 95% B from 4.0 to 5.0 min; Flow rate: 2.0 mL/min.
4. Example 63 was treated with 6 N aqueous hydrochloric acid to reveal the ketone.
5. HPLC conditions: Column: Waters XBridge C₁₈, 4.6 × 50 mm, 5 μm; Mobile phase A: 0.03% ammonium hydroxide in water (v/v); Mobile phase B: 0.03% ammonium hydroxide in acetonitrile (v/v); Gradient: 5% to 95% B over 4.0 min (linear gradient); Flow rate: 2.0 mL/min.

TABLE 13
Examples 67-68

| | |
|---|---|
| 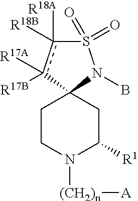 | B = 3,4-difluorophenyl R¹ = CH₃ R^{17A} = H R^{18A} = H |
| 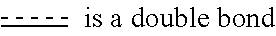 | |

| Ex # | 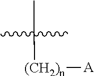 | Method of preparation; starting material(s) | IUPAC Name | ¹H NMR (400 MHz, CDCl₃), δ (ppm); Mass spectrum, observed ion m/z (M + 1) or HPLC retention time (minutes); Mass spectrum m/z (M + 1) |
|---|---|---|---|---|
| 67 | 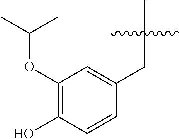 [SMILES: *Cc1ccc(O)c(OC(C)C)c1] | A; P9, P8 | 4-{[(5R,7S)-1-(3,4- difluorophenyl)-7- methyl-2,2-dioxido-2- thia-1,8- diazaspiro[4.5]dec-3- en-8-yl]methyl}-2- isopropoxyphenol | 7.20-7.27 (m, 2H), 7.13-7.17 (m, 1H), 7.09 (d, J = 7.2 Hz, 1H), 6.78- 6.83 (m, 2H), 6.71 (d, J = 1.8 Hz, 1H), 6.65 (dd, J = 8.0, 1.8 Hz, 1H), 5.65 (br s, 1H), 4.51 (septet, J = 6.1 Hz, 1H), 3.42 (AB quartet, J_{AB} = 13.3 Hz, Δν_{AB} = 119.6 Hz, 2H), 2.76-2.85 (m, 1H), 2.60 (ddd, J = 12.6, 8.4, 3.6 Hz, 1H), 2.27 (ddd, J = 12.7, 7.0, 4.0 Hz, 1H), 2.09 (dd, J = 13.6, 4.8 Hz, 1H), 1.93 (ddd, J = 13.4, 8.3, 4.0 Hz. 1H), 1.78-1.86 (m, 1H), 1.74 (ddd, J = 13.7, 6.0, 1.2 Hz, 1H), 1.33 (d, J = 6.0 Hz, 3H), 1.33 (d, J = 6.0 Hz, 3H), 1.12 (d, J = 6.6 Hz, 3H); 479.3 |
| 68 | 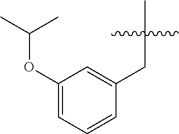 [SMILES: *Cc1cccc(OC(C)C)c1] | B; P9¹ | (5R,7S)-1-(3,4- difluorophenyl)-8-(3- isopropoxybenzyl)-7- methyl-2-thia-1,8- diazaspiro[4.5]dec-3- ene 2,2-dioxide, hydrochloride salt | 12.93 (br s, 1H), 7.26-7.31 (m, 1H), 7.08-7.14 (m, 1H), 6.99-7.05 (m, 3H), 6.85-6.89 (m, 2H), 6.67 (br s, 1H), 6.64 (br d, J = 7.6 Hz, 1H), 4.58-4.65 (m, 1H), 4.07 (br AB quartet, J_{AB} =13.6 Hz, Δν_{AB} = 33 Hz, 2H), 3.04-3.15 (m, 2H), 2.84-2.94 (m, 2H), 2.34-2.46 (m, 1H), 2.11- 2.25 (m, 2H), 1.67 (br d, J = 5.2 Hz, 3H), 1.39 (d, J = 6.0 Hz, 3H), 1.37 (d, J = 6.0 Hz, 3H); 463.3 |

1. The corresponding alcohol was converted to the requisite bromide with phosphorus tribromide.

TABLE 15
Examples 82-85

| | |
|---|---|
| 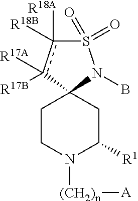 | B = 3-fluorophenyl R¹ = CH₃ R^{17A} and R^{17B} together are C═O R^{18A} = H R^{18B} = H |
| 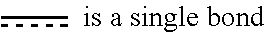 | |

| Ex # | 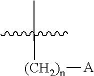 | Method of preparation¹; starting material(s) | IUPAC Name | ¹H NMR (400 MHz, CDCl₃), δ (ppm); Mass spectrum, observed ion m/z (M + 1) or HPLC retention time (minutes); Mass spectrum m/z (M + 1) |
|---|---|---|---|---|
| 82 | 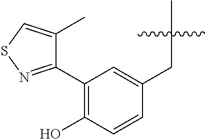 [SMILES: *Cc1ccc(O)c(-c2nscc2C)c1] | A; P6² | (5R,7S)-1-(3- fluorophenyl)-8-[4- hydroxy-3-(4- methylisothiazol-3- yl)benzyl]-7-methyl-2- thia-1,8- diazaspiro[4.5]decan- 4-one 2,2-dioxide | 11.21 (s, 1H), 8.41 (s, 1H), 7.47 (d, J = 2.0 Hz, 1H), 7.28-7.35 (m, 1H), 7.16 (d, J = 8.2 Hz, 1H), 7.04-7.13 (m, 3H), 6.98 (d, J = 8.2 Hz, 1H), 3.97 (s, 2H), 3.79 (d, J = 13.3 Hz, 1H), 3.17 (d, J = 13.5 Hz, 1H), 2.62 (dt, J = 12.2, 4.1 Hz, 1H), 2.51 (s, 3H), 2.14- 2.27 (m, 4H), 2.00-2.09 (m, 1H), 1.85-1.94 (m, 1H), 1.12 (d, J = 5.8 Hz, 3H); 516.2 |
| 83 | 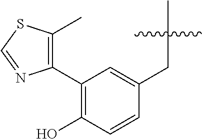 [SMILES: *Cc1ccc(O)c(-c2ncsc2C)c1] | A; P6³ | (5R,7S)-1-(3- fluorophenyl)-8-[4- hydroxy-3-(5-methyl- 1,3-thiazol-4- yl)benzyl]-7-methyl-2- thia-1,8- diazaspiro[4.5]decan- 4-one 2,2-dioxide | ¹H NMR (500 MHz, CDCl₃) δ 10.88 (br s, 1H), 8.74 (s, 1H), 7.28-7.34 (m, 2H), 7.16 (d, J = 7.8 Hz, 1H), 7.07-7.13 (m, 2H), 6.98-7.02 (m, 1H), 6.93- 6.97 (m, 1H), 3.97 (s, 2H), 3.78 (d, J = 13.7 Hz, 1H), 3.17 (d, J = 13.7 Hz, 1H), 2.66 (s, 3H), 2.60-2.65 (m, 1H), 2.11-2.27 (m, 4H), 2.01-2.09 (m, 1H), 1.86-1.94 (m, 1H), 1.13 (d, J = 6.1 Hz, 3H); 516.2 |
| 84 | 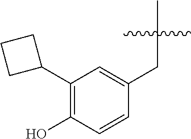 [SMILES: *Cc1ccc(O)c(C2CCC2)c1] | A; P6⁴ | (5R,7S)-8-(3- cyclobutyl-4- hydroxybenzyl)-1-(3- fluorophenyl)-7- methyl-2-thia-1,8- diazaspiro[4.5]decan- 4-one 2,2-dioxide | 7.33-7.41 (m, 1H), 7.09-7.22 (m, 4H), 6.90 (s, 1H), 6.85 (d, J = 7.8 Hz, 1H), 6.64 (d, J = 8.2 Hz, 1H), 3.97 (s, 2H), 3.70-3.80 (m, 1H), 3.57-3.69 (m, 1H), 3.02 (d, J = 12.9 Hz, 1H), 2.56-2.65 (m, 1H), 2.31-2.42 (m, 2H), 1.99- 2.24 (m, 8H), 1.79-1.92 (m, 2H), 1.11 (d, J = 5.7 Hz, 3H); 473.2 |
| 85 | 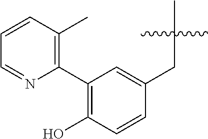 [SMILES: *Cc1ccc(O)c(-c2ncccc2C)c1] | A; P6⁵ | (5R,7S)-1-(3- fluorophenyl)-8-[4- hydroxy-3-(3- methylpyridin-2- yl)benzyl]-7-methyl-2- thia-1,8- diazaspiro[4.5]decan- 4-one 2,2-dioxide | 1.71 min⁶; 510.3 |

1. In all cases, the methyl enol ether product from the reductive amination was treated with 6 N aqueous hydrochloric acid to reveal the ketone in the Example.
2. See Table 11, footnote 22.
3. See Table 11, footnote 23.
4. See Table 11, footnote 4.
5. See Table 11, footnote 24.
6. See Table 13, footnote 5.

TABLE 16
Example 86

| | |
|---|---|
| 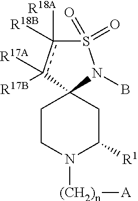 | B = 3-fluorophenyl R¹ = CH₃ R^{17A} = methoxy R^{18A} = H |
| 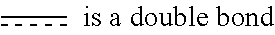 | |

| Ex # | 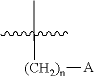 | Method of preparation; starting material(s) | IUPAC Name | HPLC retention time (minutes); Mass spectrum m/z (M + 1) |
|---|---|---|---|---|
| 86 | 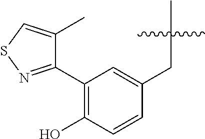 [SMILES: *Cc1ccc(O)c(-c2nscc2C)c1] | A; P6¹ | 4-{[(5R,7S)-1-(3-fluorophenyl)- 4-methoxy-7-methyl-2,2- dioxido-2-thia-1,8- diazaspiro[4.5]dec-3-en-8- yl]methyl}-2-(4- methylisothiazol-3-yl)phenol, ammonium salt | 2.36 min²; 530.3 |

1. See Table 11, footnote 22.
2. See Table 11, footnote 20.

TABLE 17
Examples 87-92

| | |
|---|---|
| 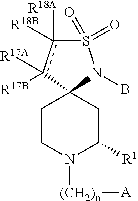 | R¹ = CH₃ R^{17A} = H R^{17B} = H R^{18A} = H R^{18B} = H |
| 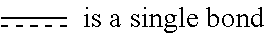 | |

| Ex # | 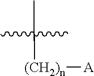 | 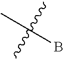 | Method of preparation; starting material(s) | IUPAC Name | ¹H NMR (400 MHz, CDCl₃), δ (ppm); Mass spectrum, observed ion m/z (M + 1) |
|---|---|---|---|---|---|
| 87 | 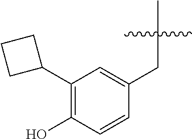 [SMILES: *Cc1ccc(O)c(C2CCC2)c1] | 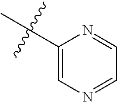 [SMILES: *c1cnccn1] | A; P11¹ | 2-cyclobutyl-4- {[(5R,7S)-7- methyl-2,2- dioxido-1- (pyrazin-2-yl)-2- thia-1,8- diazaspiro[4.5] dec-8- yl]methyl}phenol | 1.07 (d, 3H), 1.53-1.59 (m, 1H), 1.61-1.70 (m, 1H), 1.80- 1.91 (m, 1H), 1.99-2.25 (m, 4H), 2.33 (br s, 2H), 2.45- 2.70 (m, 5H), 3.26 (d, 1H), 3.41 (dd, J = 8.1, 6.9 Hz, 2H), 3.57-3.67 (m, 1H), 3.69 (s, 1H), 4.56 (br s, 1H), 6.65 (d. J = 8.2 Hz, 1H), 6.88-6.95 (m, 1H), 6.99 (s, 1H), 8.40-8.47 (m, 2H), 8.65 (s, 1H); 443.1 |
| 88 | 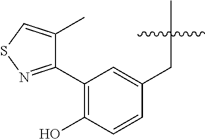 [SMILES: *Cc1ccc(O)c(-c2nscc2C)c1] | 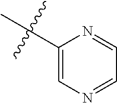 [SMILES: *c1cnccn1] | A; P11² | 4-{[(5R,7S)-7- methyl-2,2- dioxido-1- (pyrazin-2-yl)-2- thia-1,8- diazaspiro[4.5] dec-8-yl]methyl}- 2-(4- methylisothiazol- 3-yl)phenol | 1.03-1.15 (m, 3H), 1.55 (br s, 1H), 1.67 (br s, 1H), 2.26 (br s, 1H), 2.42-2.74 (m, 9H), 3.27- 3.45 (m, 3H), 3.72 (br s, 1H), 6.99 (dd, J = 8.4, 2.5 Hz, 1H), 7.09-7.19 (m, 1H), 7.57 (s, 1H), 8.33-8.47 (m, 3H), 8.68 (s, 1H), 11.12 (br s, 1H); 486.0 |
| 89 | 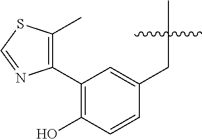 [SMILES: *Cc1ccc(O)c(-c2ncsc2C)c1] | 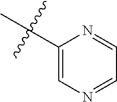 [SMILES: *c1cnccn1] | A; P11³ | 4-{[(5R,7S)-7- methyl-2,2- dioxido-1- (pyrazin-2-yl)-2- thia-1,8- diazaspiro[4.5] dec-8-yl]methyl}- 2-(5-methyl-1,3- thiazol-4- yl)phenol | 1.10 (d, J = 6.2 Hz, 3H), 1.55 (br s, 1H), 1.68 (br s, 1H), 2.20- 2.32 (m, 1H), 2.45-2.72 (m, 9H), 3.32 (d, J = 13.27 Hz, 1H), 3.40 (dd, J = 8.4, 6.8 Hz, 2H), 3.73 (d, J = 13.5 Hz, 1H), 6.95 (d, J = 8.2 Hz, 1H), 7.09 (dd, J = 8.3, 2.0 Hz, 1H), 7.40 (s, 1H), 8.37-8.45 (m, 2H), 8.67 (d, J = 1.2 Hz, 1H), 8.70 (s, 1H), 10.80 (br s, 1H); 486.0 |
| 90 | 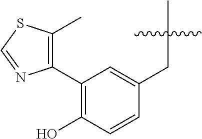 [SMILES: *Cc1ccc(O)c(-c2ncsc2C)c1] | 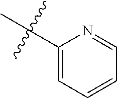 [SMILES: *c1ccccn1] | A; P10^{3,4} | 4-{[(5R,7S)-7- methyl-2,2- dioxido-1- (pyridin-2-yl)-2- thia-1,8- diazaspiro[4.5] dec-8-yl]methyl}- 2-(5-methyl-1,3- thiazol-4- yl)phenol | 1.13 (d, J = 5.9 Hz, 3H), 1.57 (br s, 1H), 1.73 (br s, 1H), 2.30 (br s, 1H), 2.39-2.72 (m, 9H), 3.31-3.41 (m, 2H), 3.54 (br s, 1H), 3.73 (d, J = 13.9 Hz, 1H), 6.91-6.97 (m, 1H), 7.03 (d, J = 8.0 Hz, 1H), 7.06-7.15 (m, 1H), 7.18-7.27 (m, 1H), 7.36 (s, 1H), 7.53-7.63 (m, 1H), 8.39 (br s, 1H), 8.71 (d, J = 1.4 Hz, 1H); 485.0 |
| 91 | 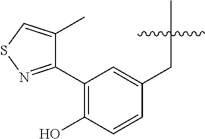 [SMILES: *Cc1ccc(O)c(-c2nscc2C)c1] | 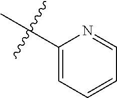 [SMILES: *c1ccccn1] | A; P10^{2,4} | 4-{[(5R,7S)-7- methyl-2,2- dioxido-1- (pyridin-2-yl)-2- thia-1,8- diazaspiro[4.5] dec-8-yl]methyl}- 2-(4- methylisothiazol- 3-yl)phenol | 1.08 (br s, 3H), 1.46-1.53 (m, 1H), 1.62-1.72 (m, 1H), 2.26 (br s, 1H), 2.41-2.51 (m, 5H), 2.51-2.65 (m, 3H), 2.65- 2.76 (m, 1H), 3.33-3.40 (m, 2H), 3.41-3.48 (m, 1H), 3.68 (br s, 1H), 6.98 (d, J = 8.4 Hz, 1H), 7.07-7.15 (m, 2H), 7.26- 7.33 (m, 1H), 7.54 (s, 1H), 7.57-7.66 (m, 1H), 8.38 (s, 1H), 8.41-8.47 (m, 1H), 11.16 (br s, 1H); 485.0 |
| 92 | 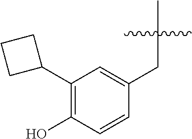 [SMILES: *Cc1ccc(O)c(C2CCC2)c1] | 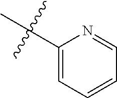 [SMILES: *c1ccccn1] | A; P10^{1,4} | 2-cyclobutyl-4- {[(5R,7S)-7- methyl-2,2- dioxido-1- (pyridin-2-yl)-2- thia-1,8- diazaspiro[4.5] dec-8- yl]methyl}phenol | 1.09 (br s, 3H), 1.66 (br s, 3H), 1.79-1.89 (m, 1H), 2.01- 2.18 (m, 3H), 2.18-2.29 (m, 1H), 2.29-2.40 (m, 2H), 2.44- 2.73 (m, 5H), 3.24-3.35 (m, 1H), 3.38 (t, J = 7.6 Hz, 2H), 3.61 (s, 1H), 3.67-3.79 (m, 1H), 6.64 (d, J = 8.0 Hz, 1H), 6.87-6.94 (m, 1H), 6.95- 7.01 (m, 1H), 7.17 (ddd, J = 7.6, 4.8, 1.1 Hz, 1H), 7.26- 7.35 (m, 1H), 7.65 (td, J = 7.7, 2.0 Hz, 1H), 8.45 (brs, 1H); 442.1 |

1. See Table 11, footnote 4.
2. See Table 11, footnote 22.
3. See Table 11, footnote 23.
4. Compound P10 was deprotected using the conditions described in step 2 of Preparation 11, to afford the secondary amine used for Method A.

TABLE 14
Biological data for Examples 1-92

| | BACE activity, 8- | BACE activity, 11- |
|---|---|---|
| | point curve, | point curve, |
| Ex # | IC₅₀ (nM)¹ | IC₅₀ (nM)¹ |
| 1 | 35.0 | N.D.³ |
| 2 | N.D. | 4440 |
| 3 | 81.5⁴ | 613 |
| 5 | 0.731 | N.D. |
| 6 | <4.2² | N.D. |
| 7 | 21.9 | N.D. |
| 8 | 124 | N.D. |
| 9 | 23.5² | 77.9 |
| 10 | 181⁴ | N.D. |
| 11 | 53.3 | N.D. |
| 12 | 338⁴ | N.D. |
| 13 | 18.4 | N.D. |
| 14 | 42.5² | N.D. |
| 15 | 754 | 478 |
| 16 | 322⁴ | N.D. |
| 17 | 84.9⁴ | 908⁴ |
| 18 | 1.86 | N.D. |
| 19 | 310⁴ | N.D. |
| 22 | 414 | N.D. |
| 23 | 428⁴ | 1450 |
| 24 | 51.9 | 353⁴ |
| 25 | 14.3 | 28.4 |
| 27 | 116⁴ | 269 |
| 28 | 521⁴ | 2160⁴ |
| 29 | 332⁴ | 1530⁴ |
| 30 | 208⁴ | N.D. |
| 31 | 10.5 | 62.6 |
| 32 | N.D. | 4290 |
| 33 | N.D. | 3030 |
| 34 | N.D. | 5890 |
| 35 | 2180⁴ | 3000 |
| 36 | 2510⁴ | 6290 |
| 37 | 1820⁴ | 2840 |
| 38 | 752⁴ | 2780 |
| 39 | 79.5⁴ | 414⁴ |
| 40 | 1380⁴ | 2380 |
| 41 | 703⁴ | 2380 |
| 42 | 117⁴ | 2220 |
| 43 | 975 | 8250 |
| 44 | 3070⁴ | 8460 |
| 45 | 5600⁴ | 15300 |
| 46 | 866 | 3010² |
| 47 | 47.1 | 249 |
| 48 | 5600 | 9030 |
| 49 | 6720⁴ | 15200 |
| 50 | 7180⁴ | 25600 |
| 51 | 2080⁴ | 5640 |
| 52 | 40.5 | N.D. |
| 53 | 2290 | 4460 |
| 54 | 53.3 | 137⁴ |
| 55 | 392⁴ | 1020⁴ |
| 56 | N.D. | 15400 |
| 57 | N.D. | 4080 |
| 58 | N.D. | 6530⁴ |
| 59 | N.D. | 8820⁴ |
| 60 | N.D. | 6540⁴ |
| 61 | <30.5⁴ | N.D. |
| 62 | 19.0 | 33.3 |
| 63 | 5820⁴ | N.D. |
| 64 | N.D. | 25700⁴ |
| 65 | N.D. | 3160 |
| 67 | 59.3 | 429 |
| 68 | 1770⁴ | 5790 |
| 69 | N.D. | 7070 |
| 70 | 2560 | 6810 |
| 71 | 1290⁴ | 2490² |
| 72 | 0.97⁴ | 24.3 |
| 73 | 2.44⁴ | 44.5 |
| 74 | 13.8⁴ | 115 |
| 75 | N.D. | 41.8 |
| 76 | 705⁴ | 2970⁴ |
| 77 | N.D. | 927 |
| 78 | N.D. | 5650 |
| 79 | 189 | 519 |
| 80 | N.D. | 6460 |
| 81 | N.D. | 5720² |
| 82 | N.D. | 20.9 |
| 83 | N.D. | 240 |
| 84 | N.D. | 337 |
| 85 | N.D. | 715 |
| 86 | N.D. | 117 |
| 87 | N.D. | 2670 |
| 88 | N.D. | 457 |
| 89 | 64.9⁴ | 1230 |
| 90 | N.D. | 3280 |
| 91 | N.D. | 763 |
| 92 | N.D. | 4190 |

¹Value represents the geometric mean of 2-4 IC₅₀ determinations, unless otherwise indicated.
²Value represents the geometric mean of 5-6 IC₅₀ determinations.
³Not determined
⁴Value represents a single IC₅₀ determination.

### Biological Assay

### BACE1 Fluorescent Polarization (FP) Assay

The fluorescently tagged synthetic substrate, Biotin-GLTNIKTEEISEISŶEVEFR-C[oregon green]KK-OH can be efficiently cleaved by the beta-secretase enzyme and is therefore useful to assay beta-secretase activity in the presence or absence of inhibitory compounds. The his tagged BACE1 enzyme was affinity purified material from conditioned media of CHO-K1 cells that had been transfected to express soluble, truncated BACE enzyme (BACE1deltaTM96His). Compounds were incubated in a ½ log dose response (from a top concentration of 100 μM) with BACE1 enzyme (0.1 nM final) and the biotinylated fluorescent peptide substrate (150 nM final) in assay buffer containing 100 mM sodium acetate, pH 4.5 (brought to pH with acetic acid), and 0.001% Tween-20. Total reaction volumes of 30 μL were carried out in 384-well black plates (Thermo Scientific 4318). Plates were covered and incubated for 3 hours at 37° C. The reactions were then stopped by addition of 30 μL of 1.5 μM Streptavidin (Pierce, 21125). After a 10 minute incubation at room temperature, plates were read on a PerkinElmer Envision for fluorescence polarization (Ex485 nm/Em530 nm). The activity of the beta-secretase enzyme is detected by changes in the fluorescence polarization (Δ mP) that occur when the substrate is cleaved by the enzyme. Incubation in the presence of an inhibitory compound demonstrates specific inhibition of beta-secretase enzymatic cleavage of the peptide substrate.

We claim:

1. A compound having the structure of formula I: 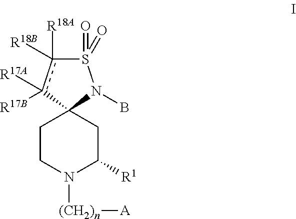
wherein the stereochemistry shown in formula I at the carbon bonded to R¹ and at the spirocyclic carbon is the absolute stereochemistry;
A is C₃₋₇cycloalkyl, C₆₋₁₀aryl, 4- to 10-membered heterocycloalkyl, or 5- to 10-membered heteroaryl; wherein said cycloalkyl, aryl, heterocycloalkyl or heteroaryl is optionally substituted with one to three R²;
R¹ is C₁₋₆alkyl, —(C(R¹⁹)₂)ₜ—C₃₋₇cycloalkyl, —(C(R¹⁹)₂)ₜ(4- to 6-membered heterocycloalkyl), —(C(R¹⁹)₂)ₜ—C₆₋₁₀aryl, or —(C(R¹⁹)₂)ₜ-(5- to 6-membered heteroaryl); wherein said alkyl, cycloalkyl, heterocycloalkyl, aryl, or heteroaryl is optionally substituted with one to three halogen, cyano, C₃₋₆cycloalkyl, hydroxyl, —O—C₁₋₆alkyl, or —O—C₃₋₆cycloalkyl;
each R² is independently C₁₋₆alkyl, halogen, cyano, —COR³, —CON(R⁴)₂, —N(R⁴)COR³, —N(R⁴)CO₂R³, —N(R⁴)CON(R⁴)₂, —N(R⁴)SO₂R³, —SO₂R³, —SO₂N(R⁴)₂, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ-(5- to 10-membered heteroaryl), —(C(R¹⁹)₂)ₜ—N(R⁴)₂, or —(C(R¹⁹)₂)ₜ—OR⁵; wherein each R² alkyl, cycloalkyl, heterocycloalkyl, aryl, or heteroaryl is optionally independently substituted by one to three cyano, C₁₋₆alkyl, halogen, C₃₋₇cycloalkyl, —CF₃ or —OR⁶;
each R³ is independently C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl); wherein each R³ alkyl, cycloalkyl, heterocycloalkyl, aryl, or heteroaryl is optionally substituted with one to three C₁₋₆alkyl, halogen, cyano, hydroxyl, or —OR⁶;
each R⁴ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5-to 10-membered heteroaryl); wherein each R⁴ alkyl, cycloalkyl, heterocycloalkyl, aryl, or heteroaryl is optionally independently substituted with one to three C₁₋₆alkyl, halogen, cyano, hydroxyl, or —OR⁶; or when two R⁴ substituents are attached to the same nitrogen atom they may be taken together with the nitrogen to which they are attached to form a 4- to 6-membered heterocycloalkyl;
each R⁵ is independently hydrogen, C₁₋₆alkyl, —(C(R¹⁹)₂)ₜ—C₃₋₇cycloalkyl, —(C(R¹⁹)₂)ₜ(4- to 10-membered heterocycloalkyl), —(C(R¹⁹)₂)ₜ—C₆₋₁₀aryl, or —(C(R¹⁹)₂)ₜ-(5- to 10-membered heteroaryl); wherein each R⁵ alkyl, cycloalkyl, heterocycloalkyl, aryl, or heteroaryl is optionally substituted with one to three R⁷;
each R⁶ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl); wherein each R⁶ alkyl, cycloalkyl, heterocycloalkyl, aryl, or heteroaryl is optionally substituted with one to three R⁸;
each R⁷ is independently C₁₋₆alkyl, hydroxyl, —O—C₁₋₆alkyl, halogen, cyano, —(C(R¹⁹)₂)ₜN(R⁹)₂, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl);
each R⁸ is independently C₁₋₆alkyl, hydroxyl, —O—C₁₋₆alkyl, halogen, cyano, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl);
each R⁹ is independently hydrogen or C₁₋₃alkyl; or when two R⁹ substituents are attached to the same nitrogen atom they may be taken together with the nitrogen to which they are attached to form a 4- to 5-membered heterocycloalkyl;
B is C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜC₆₋₁₀aryl, or —(C(R¹⁹)₂)ₜ-(5- to 10-membered heteroaryl); wherein each B alkyl, cycloalkyl, heterocycloalkyl, aryl, or heteroaryl is optionally substituted with one to three R¹⁰;
each R¹⁰ is independently halogen, C₁₋₆alkyl, cyano, hydroxyl, —O—C₁₋₆alkyl, —O—C₃₋₆cycloalkyl, —CO(C₁₋₆alkyl), —CON(R¹¹)₂, —N(R¹¹)CO(C₁₋₆alkyl), —N(R¹¹)SO₂(C₁₋₆alkyl), —SO₂(C₁₋₆alkyl), —SO₂N(R¹¹)₂, —N(R¹¹)₂, —NR¹¹CON(R¹¹)₂, —NR¹¹COOC₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl); wherein each R¹⁰ alkyl, cycloalkyl, heterocycloalkyl, aryl, or heteroaryl is optionally independently substituted with one to three R¹²;
each R¹¹ is independently hydrogen or C₁₋₆alkyl; or when two R¹¹ substituents are attached to the same nitrogen atom they may be taken together with the nitrogen to which they are attached to form a 4- to 6-membered heterocycloalkyl;
each R¹² is independently C₁₋₆alkyl, halogen, cyano, hydroxyl, —O—C₁₋₆alkyl, —O—C₃₋₆cycloalkyl, —CO(C₁₋₆alkyl), —CON(R¹¹)₂, —(C(R¹⁹)₂)ₜN(R¹³)₂, —N(R¹¹)CO(C₁₋₆alkyl), —N(R¹¹)CO₂(C₁₋₆alkyl), —NR¹¹CON(R¹¹)₂, —N(R¹¹)SO₂(C₁₋₆alkyl), —SO₂(C₁₋₆alkyl), —SO₂N(R¹¹)₂, —(C(R¹⁹)₂)ₜOR¹⁴, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜC₆₋₁₀aryl, or —(C(R¹⁹)₂)ₜ-(5- to 10-membered heteroaryl); wherein each R¹² alkyl, cycloalkyl, heterocycloalkyl, aryl, or heteroaryl is optionally independently substituted by one to three cyano, C₁₋₆alkyl, halogen, —CF₃ or —OR¹⁵;
each R¹³ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl); wherein each R¹³ alkyl, cycloalkyl, heterocycloalkyl, aryl, or heteroaryl is optionally independently substituted with one to three cyano, C₁₋₆alkyl, halogen, —CF₃, or —OR¹⁵; or when two R¹³ substituents are attached to the same nitrogen atom they may be taken together with the nitrogen to which they are attached to form a 4- to 6-membered heterocycloalkyl;
each R¹⁴ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜC₆₋₁₀aryl, or —(C(R¹⁹)₂)ₜ-(5- to 10-membered heteroaryl); wherein each R¹⁴ alkyl, cycloalkyl, heterocycloalkyl, aryl, or heteroaryl is optionally substituted with one to three cyano, C₁₋₆alkyl, halogen, —CF₃, or —OR¹⁵;
each R¹⁵ is independently hydrogen, C₁₋₆alkyl, —(C(R¹⁹)₂)ₜ—C₃₋₇cycloalkyl, —(C(R¹⁹)₂)ₜ(4- to 10-membered heterocycloalkyl), —(C(R¹⁹)₂)ₜ—C₁₋₆aryl, or —(C(R¹⁹)₂)ₜ-(5- to 10-membered heteroaryl); wherein each R¹⁵ alkyl, cycloalkyl, heterocycloalkyl, aryl, or heteroaryl is optionally substituted with one to three R⁸;
when [symbol] is a single bond, R^{17A} and R^{17B} are independently hydrogen, hydroxyl, or C₁₋₆alkyl wherein said alkyl is optionally substituted with fluorine, —SO₂(C₁₋₃alkyl), —SO₂(Cecycloalkyl), cyano, NR¹¹COO(C₁₋₃alkyl), hydroxyl, —O—C₁₋₆alkyl, or —O—C₃₋₆cycloalkyl; or R^{17A} and R^{17B} together with the carbon to which they are bonded form a C═O, C₃₋₆cycloalkyl, or 4- to 6-membered heterocycloalkyl; and R^{18A} and R^{18B} are independently hydrogen, C₁₋₆alkyl, —(C(R¹⁹)₂)ₜC₃₋₇cycloalkyl, —(C(R¹⁹)₂)ₜ-(4- to 6-membered heterocycloalkyl), —(C(R¹⁹)₂)ₜ—C₆₋₁₀aryl, —(C(R¹⁹)₂)ₜ-(5- to 6-membered heteroaryl), —(C(R¹⁹)₂)ₜ—OR¹⁶, —(C(R¹⁹)₂)ₜN(R¹¹)₂, —(C(R¹⁹)₂)ₜ—CO(C₁₋₆alkyl), —(C(R¹⁹)₂)ₜ—CON(R¹¹)₂, —(C(R¹⁹)₂)ₜ—N(R¹¹)CONR¹¹, —(C(R¹⁹)₂)ₜ—SO₂(C₁₋₆alkyl), or —(C(R¹⁹)₂)ₜ—CO₂R³; or R^{18A} and R^{18B} together with the carbon to which they are bonded form a C₃₋₆cycloalkyl or a 4- to 5-membered heterocycloalkyl, wherein said cycloalkyl or heterocycloalkyl is optionally substituted with one to two fluorine, C₁₋₆alkyl, cyano, —CF₃, C₃₋₆cycloalkyl, hydroxyl, —O—C₁₋₆alkyl, or —O—C₃₋₆cycloalkyl;
each R¹⁶ is independently hydrogen, C₁₋₃alkyl, C₃₋₅cycloalkyl, 4- to 6-membered heterocycloalkyl, C₆₋₁₀aryl, or 5- to 6-membered heteroaryl, wherein said alkyl, cycloalkyl, heterocycloalkyl, aryl, or heteroaryl is optionally substituted with one to three halogen or —CF₃;
or R^{17A} and R^{18A}, together with the carbons to which they are bonded, can form a C₃₋₆cycloalkyl or 4- to 6-membered heterocycloalkyl; wherein said cycloalkyl or heterocycloalkyl are optionally substituted with one to three C₁₋₆alkyl, fluorine, cyano, hydroxyl, —O—C₁₋₆alkyl, or —O—C₃₋₆cycloalkyl;
when [symbol] is a double bond, R^{17B} is absent and R^{17A} is hydrogen, —(C(R¹⁹)₂)ₜN(R¹⁶)₂, —(C(R¹⁹)₂)ₜ—OR¹⁶, or C₁₋₆alkyl wherein said alkyl is optionally substituted with one to three fluorine; and R^{18B} is absent and R^{18A} is hydrogen, hydroxyl, cyano, —(C(R¹⁹)₂)ₜ—C₃₋₆cycloalkyl, —(C(R¹⁹)₂)ₜ(4- to 6-membered heterocycloalkyl), —(C(R¹⁹)₂)ₜ—C₆₋₁₀aryl, —(C(R¹⁹)₂)ₜ-(5- to 6-membered heteroaryl), fluorine, C₁₋₆alkyl, —(C(R¹⁹)₂)ₜ—SO₂(C₁₋₆alkyl), —(C(R¹⁹)₂)ₜ—SO₂N(R¹¹)₂, —(C(R¹⁹)₂)ₜ—CON(R¹¹)₂, —(C(R¹⁹)₂)ₜ—COO(C₁₋₆alkyl), —(C(R¹⁹)₂)ₜ—C(O)(C₁₋₆alkyl), —(C(R¹⁹)₂)ₜ—N(R¹¹)₂, —(C(R¹⁹)₂)ₜ—NR¹¹CO(C₁₋₆alkyl), —(C(R₁₉)ₜ—N(R¹¹)CO₂(C₁₋₆alkyl), —(C(R¹⁹)₂)ₜ—NR¹¹CON(R¹¹)₂, or —(C(R¹⁹)₂)ₜ—N(R¹¹)SO₂(C₁₋₆alkyl); wherein said alkyl, cycloalkyl, heterocycloalkyl, aryl or heteroaryl substituent is optionally substituted with one to three halogen, cyano, —CF₃, C₁₋₆alkyl, hydroxyl, —O—C₁₋₆alkyl, or —O—C₃₋₆cycloalkyl;
or R^{17A} and R^{18A}, together with the carbons to which they are bonded, can form a fused C₅₋₆cycloalkyl, 5- to 6-membered heterocycloalkyl, 6- to 10-membered aryl or a 5- to 6-membered heteroaryl ring; wherein said cycloalkyl, heterocycloalkyl, aryl, or heteroaryl are optionally substituted with one to three C₁₋₆alkyl, halogen, cyano, —CF₃, hydroxyl, —O—C₁₋₆alkyl, or —O—C₃₋₆cycloalkyl;
each R19 is independently hydrogen, C₁₋₆alkyl, or CF₃;
n is an integer independently selected from 1, 2 and 3;
each t is an integer independently selected from 0, 1, 2 and 3; and pharmaceutically acceptable salts thereof.

2. A compound of claim 1 wherein A is C₃₋₇cycloalkyl, C₆₋₁₀aryl, 4- to 10-membered heterocycloalkyl, or 5- to 10-membered heteroaryl and A is optionally substituted with one to three R² substituents selected from the group consisting of C₁₋₆alkyl, halogen, cyano, —COR³, —CON(R⁴)₂, —N(R⁴)COR³, —N(R⁴)CO₂R³, —N(R⁴)CON(R⁴)₂, —N(R⁴)SO₂R³, —SO₂R³, —SO₂N(R⁴)₂, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ-(5- to 10-membered heteroaryl), —(C(R¹⁹)₂)ₜ—N(R⁴)₂, or —(C(R¹⁹)₂)ₜ—OR⁵; wherein each R² alkyl, cycloalkyl, heterocycloalkyl, aryl, or heteroaryl is optionally substituted by one to three cyano, C₁₋₆alkyl, C₃₋₇cycloalkyl, halogen, —CF₃ or —OR⁶; and pharmaceutically acceptable salts thereof.

3. A compound of claim 2 wherein A is C₆₋₁₀aryl and A is optionally substituted with one to three R² substituents selected from the group consisting of C₁₋₆alkyl, halogen, cyano, —COR³, —CON(R⁴)₂, —(C(R¹⁹)₂)ₜC₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ-(5- to 10-membered heteroaryl), or —(C(R¹⁹)₂)ₜ—OR⁵; wherein each R² alkyl, cycloalkyl, heterocycloalkyl, aryl, or heteroaryl is optionally substituted by one to three cyano, C₁₋₆alkyl, C₃₋₇cycloalkyl, halogen, —CF₃ or —OR⁶; and pharmaceutically acceptable salts thereof.

4. A compound of claim 3 wherein t is 0 or 1; B is —(C(R¹⁹)₂)ₜ—C₆₋₁₀aryl, or —(C(R¹⁹)₂)ₜ-(5- to 10-membered heteroaryl); wherein each aryl, or heteroaryl is optionally substituted with one to three R¹⁰ and R¹⁰ is independently selected from halogen, C₁₋₆alkyl, cyano, hydroxyl, —CF₃, —O—C₁₋₆alkyl, —O—C₃₋₆cycloalkyl; wherein each R¹⁰ alkyl, cycloalkyl is optionally independently substituted with one to three R¹²; and pharmaceutically acceptable salts thereof.

5. A compound of claim 4 wherein R² is selected from chloro, fluoro, hydroxyl, —CF₃, cyclopropyl, cyclobutyl, cyclopentyl, cyclohexyl, —O(C₁₋₆alkyl), —O(C₃₋₇cycloalkyl), thiophene, phenyl, O—CF₃, tetrahydrofuran, —CH₂(C₃₋₇cycloalkyl), pyridine, pyrimidine, thiazole, oxazole, isoxazole oxazole, isoxazole, isothiazole or pyranyl, said R² optionally substituted with one to three methyl, ethyl, isopropyl chloro, fluoro, alkoxy, C₃₋₇cycloalkyl or hydroxyl; and pharmaceutically acceptable salts thereof.

6. A compound according to claim 2 wherein A is 5- to 10-membered heteroaryl, and A is optionally substituted with one to three R² substituents selected from the group consisting of halogen, C₁₋₆alkyl, cyano, —COR³, —(C(R¹⁹)₂)ₜ—OR⁵, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜC₆₋₁₀aryl, or —(C(R¹⁹)₂)ₜ-(5- to 10-membered heteroaryl); wherein said cycloalkyl, heterocycloalkyl, aryl, or heteroaryl is optionally substituted by one to three C₁₋₆alkyl, C₃₋₇cycloalkyl, —CF₃, alkoxy or halogen; and pharmaceutically acceptable salts thereof.

7. A compound according to claim 6 wherein t is 0 to 1; B is —(C(R¹⁹)₂)ₜ—C₆₋₁₀aryl, or —(C(R¹⁹)₂)ₜ-(5- to 10-membered heteroaryl); wherein each aryl, or heteroaryl is optionally substituted with one to two R¹⁰ and R¹⁰ is independently selected from fluoro and chloro; and pharmaceutically acceptable salts thereof.

8. A compound of claim 7 wherein R² is selected from chloro, fluoro, hydroxyl, —CF₃, cyclopropyl, cyclobutyl, cyclopentyl, cyclohexyl, —O(C₁₋₆alkyl), —O(C₃₋₇cycloalkyl), thiophene, phenyl, O—CF₃, tetrahydrofuran, —CH₂(C₃₋₇cycloalkyl), pyridine, pyrimidine, thiazole, isothiazole or pyran, said R² optionally substituted with one to three methyl, ethyl, isopropyl chloro, fluoro, oxazole, isooxazole or hydroxyl; and pharmaceutically acceptable salts thereof.

9. A compound having the structure of formula Ia: 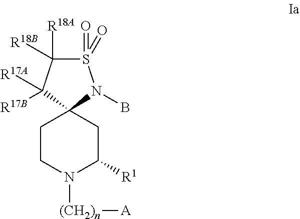
wherein the stereochemistry shown in formula Ia at the carbon bonded to R¹ and at the spirocyclic carbon is the absolute stereochemistry;
A is C₆₋₁₀aryl or 5- to 10-membered heteroaryl; wherein said aryl or heteroaryl is optionally substituted with one to three R²;
R¹ is C₁₋₆alkyl; wherein said alkyl is optionally substituted with one to three halogen, cyano, C₃₋₆cycloalkyl, hydroxyl, —O—C₁₋₆alkyl, or —O—C₃₋₆cycloalkyl;
each R² is independently C₁₋₆alkyl, halogen, cyano, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ-(5- to 10-membered heteroaryl) or —(C(R¹⁹)₂)ₜ—OR⁵; wherein each R² alkyl, cycloalkyl, heterocycloalkyl, aryl, or heteroaryl is optionally independently substituted by one to three cyano, C₁₋₆alkyl, halogen, C3-eCycloalkyl, —CF₃ or —OR⁶;
each R⁵ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl); wherein each R⁵ alkyl, cycloalkyl, heterocycloalkyl, aryl, or heteroaryl is optionally substituted with one to three R⁷;
each R⁶ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl); wherein each R⁶ alkyl, cycloalkyl, heterocycloalkyl, aryl, or heteroaryl is optionally substituted with one to three R⁸;
each R⁷ is independently C₁₋₆alkyl, hydroxyl, —O—C₁₋₆alkyl, halogen, cyano, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl);
each R⁸ is independently C₁₋₆alkyl, hydroxyl, —O—C₁₋₆alkyl, halogen, cyano, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl);
B is —(C(R¹⁹)₂)ₜ—C₆₋₁₀aryl, or —(C(R¹⁹)₂)ₜ-(5- to 10-membered heteroaryl); wherein each B aryl or heteroaryl is optionally substituted with one to three R¹⁰;
each R¹⁰ is independently halogen, C₁₋₆alkyl, cyano, hydroxyl, —CF₃, —O—C₁₋₆alkyl, —O—C₃₋₆cycloalkyl; wherein each R¹⁰ alkyl, cycloalkyl is optionally independently substituted with one to three R¹²;
each R¹² is independently C₁₋₆alkyl, halogen, cyano, hydroxyl,
R^{17A} and R^{17B} are independently hydrogen, hydroxyl, or C₁₋₆alkyl wherein said alkyl is optionally substituted with fluorine, cyano, hydroxyl, —O—C₁₋₆alkyl, or —O—C₃₋₆cycloalkyl; or R^{17A} and R^{17B} together with the carbon to which they are bonded form a C═O, C₃₋₆cycloalkyl, or 4- to 6-membered heterocycloalkyl; and R^{18A} and R^{18B} are independently hydrogen, C₁₋₆alkyl, —(C(R¹⁹)₂)ₜ—C₃₋₇cycloalkyl, —(C(R¹⁹)₂)ₜ—OR¹⁶; or R^{18A} and R^{18B} together with the carbon to which they are bonded form a C₃₋₆cycloalkyl or a 4- to 5-membered heterocycloalkyl, wherein said cycloalkyl or heterocycloalkyl is optionally substituted with one to two fluorine, C₁₋₆alkyl, cyano, —CF₃, C₃₋₆cycloalkyl, hydroxyl, —O—C₁₋₆alkyl;
each R¹⁶ is independently hydrogen, C₁₋₃alkyl or C₃₋₅cycloalkyl, wherein said alkyl or cycloalkyl is optionally substituted with one to three halogen or —CF₃;
or R^{17A} and R^{18A}, together with the carbons to which they are bonded, can form a C₃₋₆cycloalkyl or 4- to 6-membered heterocycloalkyl; wherein said cycloalkyl or heterocycloalkyl are optionally substituted with one to three C₁₋₆alkyl, fluorine, cyano, hydroxyl, —O—C₁₋₆alkyl, or —O—C₃₋₆cycloalkyl;
each R19 is independently hydrogen, C₁₋₆alkyl, or CF₃;
n is an integer independently selected from 1, 2 and 3;
each t is an integer independently selected from 0, 1, 2 and 3; and pharmaceutically acceptable salts thereof.

10. A compound of claim 9 wherein R¹ is methyl, t is 0 or 1, B is phenyl, pyridinyl or pyrazinyl are optionally substituted with one to two fluorine, C₁₋₆alkyl, cyano, —CF₃, C₃₋₆cycloalkyl, hydroxyl, —O—C₁₋₆alkyl; or pharmaceutically acceptable salts thereof.

11. A compound of claim 10, wherein n is 1; A is phenyl, oxazolyl, pyridinyl, thiazolyl or indolyl; wherein said phenyl, oxazolyl, pyridinyl, thiazolyl or indolyl is optionally substituted with one to three R²;
or pharmaceutically acceptable salts thereof.

12. A compound of claim 11, wherein A is optionally substituted with one to three R² substituents independently selected from chloro, fluoro, hydroxyl, —CF₃, cyclopropyl, cyclobutyl, cyclopentyl, cyclohexyl, —O(C₁₋₆alkyl), —O(C₃₋₇cycloalkyl), thiophenyl, phenyl, O—CF₃, tetrahydrofuranyl, —CH₂(C₃₋₇cycloalkyl), pyridinyl, pyrimidinyl, thiophenyl, thiazolyl, isothiazolyl, isoxazolyl, oxazolyl or pyranyl, said R² optionally substituted with one to three methyl, ethyl, isopropyl chloro, fluoro, alkoxy, C₃₋₆cycloalkyl or hydroxyl; and pharmaceutically acceptable salts thereof.

13. A compound of claim 12 wherein B is optionally substituted and independently substituted with 1 to 2 fluoro or methyl.

14. A compound having the structure of formula Ib: 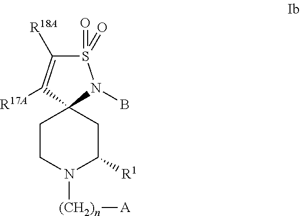
wherein the stereochemistry shown in formula Ia at the carbon bonded to R¹ and at the spirocyclic carbon is the absolute stereochemistry;
A is C₆₋₁₀aryl or 5- to 10-membered heteroaryl; wherein said aryl or heteroaryl is optionally substituted with one to three R²;
R¹ is C₁₋₆alkyl; wherein said alkyl is optionally substituted with one to three halogen, cyano, C₃₋₆cycloalkyl, hydroxyl, —O—C₁₋₆alkyl, or —O—C₃₋₆cycloalkyl;
each R² is independently C₁₋₆alkyl, halogen, cyano, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, —(C(R¹⁹)₂)ₜ-(5- to 10-membered heteroaryl) or —(C(R¹⁹)₂)ₜ—OR⁵; wherein each R² alkyl, cycloalkyl, heterocycloalkyl, aryl, or heteroaryl is optionally independently substituted by one to three cyano, C₁₋₆alkyl, halogen, C₃₋₇cycloalkyl, —CF₃ or —OR⁶;
each R⁵ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl or —(C(R¹⁹)₂)ₜ-(5- to 10-membered heteroaryl); wherein each R⁵ alkyl, cycloalkyl, heterocycloalkyl, aryl, or heteroaryl is optionally substituted with one to three R⁷;
each R⁶ is independently hydrogen, C₁₋₆alkyl, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl); wherein each R⁶ alkyl, cycloalkyl, heterocycloalkyl, aryl, or heteroaryl is optionally substituted with one to three R⁸;
each R⁷ is independently C₁₋₆alkyl, hydroxyl, —O—C₁₋₆alkyl, halogen, cyano, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl);
each R⁸ is independently C₁₋₆alkyl, hydroxyl, —O—C₁₋₆alkyl, halogen, cyano, —(C(R¹⁹)₂)ₜ—C₃₋₇cycloalkyl, —(C(R¹⁹)₂)ₜ-(4- to 10-membered heterocycloalkyl), —(C(R¹⁹)₂)ₜ—C₆₋₁₀aryl, or —(C(R¹⁹)₂)ₜ-(5- to 10-membered heteroaryl);
B is —(C(R¹⁹)₂)ₜ—C₆₋₁₀aryl, or —(C(R¹⁹)₂)ₜ-(5- to 10-membered heteroaryl); wherein each B aryl or heteroaryl is optionally substituted with one to three R¹⁰;
each R¹⁰ is independently halogen, C₁₋₆alkyl, cyano, hydroxyl, —O—C₁₋₆alkyl, —O—C₃₋₆cycloalkyl; wherein each R¹⁰ alkyl, cycloalkyl is optionally independently substituted with one to three R¹²;
each R¹² is independently C₁₋₆alkyl, halogen, cyano, hydroxyl,
R^{17A} is hydrogen, —(C(R¹⁹)₂)ₜN(R¹⁶)₂, —(C(R¹⁹)₂)ₜ—OR¹⁶, or C₁₋₆alkyl wherein said alkyl is optionally substituted with one to three fluorine; and R^{18A} is hydrogen, hydroxyl, cyano, —(C(R¹⁹)₂)ₜ—C₃₋₆cycloalkyl, —(C(R¹⁹)₂)ₜ-(4- to 6-membered heterocycloalkyl), —(C(R¹⁹)₂)ₜ—C₆₋₁₀aryl, —(C(R¹⁹)₂)ₜ-(5- to 6-membered heteroaryl), fluorine, C₁₋₆alkyl; wherein said alkyl, cycloalkyl, heterocycloalkyl, aryl or heteroaryl substituent is optionally substituted with one to three halogen, cyano, —CF₃, C₁₋₆alkyl, hydroxyl, —O—C₁₋₆alkyl, or —O—C₃₋₆cycloalkyl;
or R^{17A} and R^{18A}, together with the carbons to which they are bonded, can form a fused C₅₋₈cycloalkyl, 5- to 6-membered heterocycloalkyl, 6- to 10-membered aryl or a 5- to 6-membered heteroaryl ring; wherein said cycloalkyl, heterocycloalkyl, aryl, or heteroaryl are optionally substituted with one to three C₁₋₆alkyl, halogen, cyano, —CF₃, hydroxyl, —O—C₁₋₆alkyl, or —O—C₃₋₆cycloalkyl;
each R19 is independently hydrogen, C₁₋₆alkyl, or CF₃;
n is an integer independently selected from 1, 2 and 3;
each t is an integer independently selected from 0, 1, 2 and 3; and pharmaceutically acceptable salts thereof.

15. A compound of claim 14 wherein R¹ is methyl, t is 0 or 1, B is phenyl, pyridinyl or pyrazinyl; wherein said phenyl, pyridinyl or pyrazinyl is optionally substituted with one to three R¹⁰;
or pharmaceutically acceptable salts thereof.

16. A compound of claim 15, wherein n is 1; A is phenyl, oxazolyl, pyridinyl, thiazblyl or indolyl; wherein said phenyl, oxazolyl, pyridinyl, thiazolyl or indolyi is optionally substituted with one to three R²;
or pharmaceutically acceptable salts thereof.

17. A compound of claim 16, wherein A is optionally substituted with one to three R² substituents independently selected from chloro, fluoro, hydroxyl, —CF₃, cyclopropyl, cyclobutyl, cyclopentyl, cyclohexyl, —O(C₁₋₆alkyl), —O(C₃₋₇cycloalkyl), thiophenyl, phenyl, O—CF₃, tetrahydrofuran, —CH₂(C₃₋₇cycloalkyl), pyridinyl, pyrimidinyl, thiazolyl, isothiazolyl, isoxazolyl, oxazolyl or pyranyl, said R² optionally substituted with one to three methyl, ethyl, isopropyl chloro, fluoro, alkoxy, cyclopropyl, —CF₃ or hydroxyl; and pharmaceutically acceptable salts thereof.

18. A compound of claim 17 wherein B is optionally substituted and independently substituted with 1 to 2 fluoro or methyl; or pharmaceutically acceptable salts thereof.

19. A method for the treatment of a disease or condition selected from the group consisting of neurological and psychiatric disorders comprising administering to the mammal an effective amount of compound of claim 1 or pharmaceutically acceptable salt thereof.

20. A method according to claim 18 wherein the neurological disorder is Alzheimer disease.

21. A pharmaceutical composition comprising a compound of claim 1 or a pharmaceutically acceptable salt thereof and a pharmaceutically acceptable carrier.

22. The composition of claim 21 further comprising the administration of an atypical antipsychotic, a cholinesterase inhibitor, dimebon or NMDA receptor antagonist in combination with the compounds of claim 1.

23. A compound selected from the group consisting of:
4-{[(5R,7S)-1-(3-Fluorophenyl)-7-methyl-2,2-dioxido-2-thia-1,8-diazaspiro[4.5]dec-8-yl]methyl}-2-isopropoxyphenol;
6-{[(5R,7S)-1-(3-Fluorophenyl)-7-methyl-2,2-dioxido-2-thia-1,8-diazaspiro[4.5]dec-8-yl]methyl}-4-isopropoxypyridin-3-ol;
4-{[(5R,7S)-1-(3-fluorophenyl)-7-methyl-2,2-dioxido-2-thia-1,8-diazaspiro[4.5]dec-3-en-8-yl]methyl}-2-(3-methyl-2-thienyl)phenol, hydrochloride salt;
2′-ethyl-5-{[(5R,7S)-1-(3-fluorophenyl)-7-methyl-2,2-dioxido-2-thia-1,8-diazaspiro[4.5]dec-3-en-8-yl]methyl}biphenyl-2-ol;
2-cyclopentyl-4-{[(5R,7S)-1-(3-fluorophenyl)-7-methyl-2,2-dioxido-2-thia-1,8-diazaspiro[4.5]dec-3-en-8-yl]methyl}phenol;
2′-ethyl-5-{[(5R,7S)-1-(3-fluorophenyl)-7-methyl-2,2-dioxido-2-thia-1,8-diazaspiro[4.5]dec-8-yl]methyl}biphenyl-2-ol;
4-{[(5R,7S)-1-(3-fluorophenyl)-7-methyl-2,2-dioxido-2-thia-1,8-diazaspiro[4.5]dec-3-en-8-yl]methyl}-2-isopropoxyphenol;
4-{[(5R,7S)-1-(3-fluorophenyl)-7-methyl-2,2-dioxido-2-thia-1,8-diazaspiro[4.5]dec-3-en-8-yl]methyl}-2-(trifluoromethoxy)phenol, hydrochloride salt;
4-{[(5R,7S)-1-(3-fluorophenyl)-7-methyl-2,2-dioxido-2-thia-1,8-diazaspiro[4.5]dec-3-en-8-yl]methyl}-2-(tetrahydrofuran-2-yl)phenol;
(5R,7S)-1-(3-fluorophenyl)-8-[(5-isobutyl-1,3-oxazol-4-yl)methyl]-7-methyl-2-thia-1,8-diazaspiro[4.5]dec-3-ene 2,2-dioxide;
2-(cyclopropyloxy)-4-{[(5R,7S)-1-(3-fluorophenyl)-7-methyl-2,2-dioxido-2-thia-1,8-diazaspiro[4.5]dec-3-en-8-yl]methyl}phenol, hydrochloride salt;
2-(cyclopropyloxy)-4-{[(5R,7S)-1-(3-fluorophenyl)-7-methyl-2,2-dioxido-2-thia-1,8-diazaspiro[4.5]dec-3-en-8-yl]methyl}phenol;
2-chloro-4-{[(5R,7S)-1-(3-fluorophenyl)-7-methyl-2,2-dioxido-2-thia-1,8-diazaspiro[4.5]dec-3-en-8-yl]methyl}phenol, hydrochloride salt;
4-{[(5R,7S)-1-(3-fluorophenyl)-7-methyl-2,2-dioxido-2-thia-1,8-diazaspiro[4.5]dec-3-en-8-yl]methyl}-2-(trifluoromethyl)phenol, hydrochloride;
2-fluoro-4-{[(5R,7S)-1-(3-fluorophenyl)-7-methyl-2,2-dioxido-2-thia-1,8-diazaspiro[4.5]dec-3-en-8-yl]methyl}phenol, hydrochloride salt;
(5R,7S)-8-{[4-(cyclobutylmethyl)-1,3-thiazol-5-yl]methyl}-1-(3-fluorophenyl)-7-methyl-2-thia-1,8-diazaspiro[4.5]dec-3-ene 2,2-dioxide;
(5R,7S)-1-(3-fluorophenyl)-7-methyl-8-[(5-pyridin-3-yl-1,3-oxazol-4-yl)methyl]-2-thia-1,8-diazaspiro[4.5]dec-3-ene 2,2-dioxide, hydrochloride salt;
(5R,7S)-8-{[4-(cyclopropylmethyl)-1,3-thiazol-5-yl]methyl}-1-(3-fluorophenyl)-7-methyl-2-thia-1,8-diazaspiro[4.5]dec-3-ene-2,2-dioxide, hydrochloride salt;
5-{[(5R,7S)-1-(3-fluorophenyl)-7-methyl-2,2-dioxido-2-thia-1,8-diazaspiro[4.5]dec-3-en-8-yl]methyl}-2′-methylbiphenyl-2-ol, hydrochloride salt;
2-ethoxy-4-{[(5R,7S)-1-(3-fluorophenyl)-7-methyl-2,2-dioxido-2-thia-1,8-diazaspiro[4.5]dec-3-en-8-yl]methyl}phenol;
4-{[(5R,7S)-1-(3-fluorophenyl)-7-methyl-2,2-dioxido-2-thia-1,8-diazaspiro[4.5]dec-3-en-8-yl]methyl}-2-(tetrahydrofuran-2-yl)phenol;
4-{[(5R,7S)-1-(3-fluorophenyl)-7-methyl-2,2-dioxido-2-thia-1,8-diazaspiro[4.5]dec-3-en-8-yl]methyl}-2-(tetrahydrofuran-2-yl)phenol;
4-{[(5R,7S)-1-(3-fluorophenyl)-7-methyl-2,2-dioxido-2-thia-1,8-diazaspiro[4.5]dec-8-yl]methyl}-2-(tetrahydrofuran-2-yl)phenol;
(5R,7S)-1-(3-fluorophenyl)-7-methyl-8-[(2′-methylbiphenyl-3-yl)methyl]-2-thi diazaspiro[4.5]decane 2,2-dioxide, formate salt;
(5R,7S)-1-(3-fluorophenyl)-7-methyl-8-[(2′-methylbiphenyl-3-yl)methyl]-2-thia-1,8-diazaspiro[4.5]dec-3-ene 2,2-dioxide;
4-{[(5R,7S)-1-(3-fluorophenyl)-7-methyl-2,2-dioxido-2-thia-1,8-diazaspiro[4.5]dec-8-yl]methyl}-2-(tetrahydrofuran-2-yl)phenol;
5-{[(5R,7S)-1-(3-fluorophenyl)-7-methyl-2,2-dioxido-2-thia-1,8-diazaspiro[4.5]dec-8-yl]methyl}-2′-methylbiphenyl-2-ol, hydrochloride salt;
4-{[(5R,7S)-1-(3,4-difluorophenyl)-7-methyl-2,2-dioxido-2-thia-1,8-diazaspiro[4.5]dec-3-en-8-yl]methyl}-2-isopropoxyphenol;
4-{[(5R,7S)-1-(3-fluorophenyl)-7-methyl-2,2-dioxido-2-thia-1,8-diazaspiro[4.5]dec-3-en-8-yl]methyl}-2-(4-methyl-1,2-thiazol-3-yl)phenol, trifluoroacetic acid salt;
4-{[(5R,7S)-1-(3-fluorophenyl)-7-methyl-2,2-dioxido-2-thia-1,8-diazaspiro[4.5]dec-3-en-8-yl]methyl}-2-(5-methyl-1,3-thiazol-4-yl)phenol, ammonium salt;
4-{[(5R,7S)-1-(3-fluorophenyl)-7-methyl-2,2-dioxido-2-thia-1,8-diazaspiro[4.5]dec-3-en-8-yl]methyl}-2-(3-methylpyridin-2-yl)phenol;
2-cyclobutyl-4-{[(5R,7S)-1-(3-fluorophenyl)-7-methyl-2,2-dioxido-2-thia-1,8-diazaspiro[4.5]dec-3-en-8-yl]methyl}phenol;
(5R,7S)-1-(3-fluorophenyl)-8-[4-hydroxy-3-(4-methylisothiazol-3-yl)benzyl]-7-methyl-2-thia-1,8-diazaspiro[4.5]decan-4-one 2,2-dioxide;
(5R,7S)-1-(3-fluorophenyl)-8-[4-hydroxy-3-(5-methyl-1,3-thiazol-4-yl)benzyl]-7-methyl-2-thia-1,8-diazaspiro[4.5]decan-4-one 2,2-dioxide;
(5R,7S)-8-(3-cyclobutyl-4-hydroxybenzyl)-1-(3-fluorophenyl)-7-methyl-2-thia-1,8-diazaspiro[4.5]decan-4-one 2,2-dioxide;
4-{[(5R,7S)-1-(3-fluorophenyl)-4-methoxy-7-methyl-2,2-dioxido-2-thia-1,8-diazaspiro[4.5]dec-3-en-8-yl]methyl}-2-(4-methylisothiazol-3-yl)phenol, ammonium salt; and
4-{[(5R,7S)-7-methyl-2,2-dioxido-1-(pyrazin-2-yl)-2-thia-1,8-diazaspiro[4.5]dec-8-yl]methyl}-2-(4-methylisothiazol-3-yl)phenol;
or pharmaceutically acceptable salts thereof.
